(19) United States
(12) Patent Application Publication
(10) Pub. No.: US 2024/0166710 A1
(43) Pub. Date: May 23, 2024

(54) METHODS OF ADMINISTERING LONG-ACTING GROWTH HORMONE POLYPEPTIDES

(71) Applicants: Pfizer Inc., New York, NY (US); OPKO Biologics Ltd., Kiryat Gat (IL)

(72) Inventors: Jóse Francisco Cara, Norwalk, CT (US); Aleksandra Pastrak, Toronto (CA); Srinivas Rao Valluri, Hackettstown, NJ (US); Michael Paul Wajnrajch, Merrick, NY (US)

(73) Assignees: Pfizer Inc., New York, NY (US); OPKO Biologics Ltd., Kiryat Gat (IL)

(21) Appl. No.: 18/282,844

(22) PCT Filed: Mar. 17, 2022
(86) PCT No.: PCT/US2022/020804

§ 371 Date: Sep. 19, 2023

Related U.S. Application Data
(60) Provisional application No. 63/272,417, filed on Oct. 27, 2021.
(60) Provisional application No. 63/163,504, filed on Mar. 19, 2021.

(51) Int. Cl. C07K 14/61; A61P 5/06; G01N 33/74

(52) U.S. Cl. CPC C07K 14/61; A61P 5/06; G01N 33/74; G01N 2333/65; G01N 2800/52

(57) ABSTRACT

The subject matter described herein is directed to methods of treating growth hormone related disorders by administering a long-acting recombinant human growth hormone. In another embodiment, a long-acting recombinant human growth hormone is administered in a composition or the combination is administered separately to treat growth deficiency in a subject previously treated with a once daily rhGH therapy.

### CROSS REFERENCE TO RELATED APPLICATIONS AND INCORPORATION OF SEQUENCE LISTING

This application claims the benefit of U.S. Provisional Application No. 63/163,504, filed Mar. 19, 2021, and U.S. Provisional Application No. 63/272,417, filed Oct. 27, 2021, each of which is herein incorporated by reference in their entireties. A sequence listing contained in the file named “P35161WO00 SL. TXT” which is 5,121 bytes (measured in MS-Windows®) and created on Mar. 14, 2022, is filed electronically herewith and incorporated by reference in its entirety.

### FIELD

The present disclosure relates the use of a long-acting recombinant human growth hormone for treatment of growth hormone-related disorders such as, for example, growth hormone deficiency, and the growth failure seen in children born small for gestational age, Turner syndrome and idiopathic short stature.

### BACKGROUND

Polypeptides are susceptible to denaturation or enzymatic degradation in the blood, liver, or kidney. Accordingly, polypeptides typically have short circulatory half-lives of several hours. Because of their low stability, peptide drugs are usually delivered in a sustained frequency so as to maintain an effective plasma concentration of the active peptide. Moreover, since peptide drugs are usually administered by infusion, frequent injection of peptide drugs causes considerable discomfort to a subject.

Unfavorable pharmacokinetics, such as a short serum half-life, can prevent the pharmaceutical development of many otherwise promising drug candidates. Serum half-life is an empirical characteristic of a molecule and must be determined experimentally for each new potential drug. For example, with lower molecular weight polypeptide drugs, physiological clearance mechanisms such as renal filtration can make the maintenance of therapeutic levels of a drug unfeasible because of cost or frequency of the required dosing regimen. Conversely, a long serum half-life is undesirable where a drug or its metabolites have toxic side effects.

Thus, there is a need for technologies that will prolong the half-lives of therapeutic polypeptides while maintaining a high pharmacological efficacy thereof. Such desired peptide drugs should also meet the requirements of enhanced serum stability, high activity and a low probability of inducing an undesired immune response when injected into a subject. The present invention addresses this need by providing CTP-modified peptides having prolonged half-lives while maintaining a high pharmacological efficacy, and while having enhanced serum stability, high activity and low probability of inducing undesired immune responses in a subject.

### SUMMARY

In some embodiments of the disclosure, a method of treating growth hormone deficiency in a subject in need thereof comprises i) administering a long-acting recombinant human growth hormone (rhGH) comprising the amino acid sequence of SEQ ID NO:2 to the subject at an initial dose level; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; and iii) administering the long-acting rhGH to the subject at a modified dose level wherein the modified dose level is about 15% lower than the initial dose level. In some embodiments, a subject with an SDS>+2 is a subject whose serum IGF-1 concentration exceeds the mean reference value for their age and sex by more than 2 SDS.

In some embodiments of the disclosure, a method of treating growth hormone deficiency in a subject in need thereof comprises i) administering a long-acting recombinant human growth hormone (rhGH) comprising the amino acid sequence of SEQ ID NO:2 to the subject at an initial dose level of about 0.66 mg per kg body weight per week; ii) taking at least two measurements of an IGF-1 level in the subject at day 3 to day 4 after administering the long-acting rhGH, wherein the IGF-1 level in the subject on two consecutive measurements taken 4 to 6 weeks apart each has a standard deviation score (SDS) of >+2; iii) administering the long-acting rhGH to the subject at a modified dose level, wherein the modified dose level is 15% lower than the initial dose level; iv) taking at least one measurement of an IGF-1 level in the subject at least 4 weeks after administering the modified dose level, wherein the IGF-1 level at least 4 weeks after administering the modified dose level has a SDS of >+2; and v) administering the long-acting rhGH to the subject at a further modified dose level, wherein the further modified dose level is 15% lower than the modified dose level.

In some embodiments of the disclosure, a method of treating growth hormone deficiency in an adult subject in need thereof comprises: i) administering a long-acting recombinant human growth hormone (rhGH) comprising the amino acid sequence of SEQ ID NO:2 to the subject at an initial dose level; ii) taking at least one measurement of an IGF-1 level in the subject wherein the IGF-1 level in the subject has a standard deviation score (SDS) of >+1.5 or <−0.5; and iii) administering the long-acting rhGH to the subject at a modified dose level, wherein the modified dose level is about 0.5 mg/week or about 0.75 mg/week lower than the initial dose level when the IGF-1 level is the subject has an SDS value of >+1.5, or wherein the modified dose level is about 1.0 mg/week or about 1.5 mg/week higher than the initial dose level when the IGF-1 level in the subject has an SDS of <−0.5.

In some embodiments of the disclosure, a method of treating growth hormone deficiency in an adult subject in need thereof comprises i) administering a long-acting recombinant human growth hormone (rhGH) comprising the amino acid sequence of SEQ ID NO:2 to the subject at an initial dose level of about 1 mg/week to about 5 mg/week; ii) taking at least one measurement of an IGF-1 level in the subject at day 3 to day 4 after administering the long-acting rhGH, wherein the IGF-1 level in the subject has an SDS of <−0.5 or >+1.5; iii) administering the long-acting rhGH to the subject at a modified dose level, wherein the modified dose level is about 0.5 mg/week or about 0.75 mg/week lower than the initial dose level if the IGF-1 level in the subject has an SDS of >+1.5 or wherein the modified dose level is about 1.0 mg/week or about 1.5 mg/week higher than the initial dose level if the IGF-1 level in the subject has an SDS of <−0.5; and optionally iv) taking a measurement of the IGF-1 level in the subject after administering the modified dose level, wherein the IGF-1 level after administering the modified dose level has an SDS of <−0.5 or >+1.5; and v) administering the long-acting rhGH to the subject at a further modified dose level, wherein the further modified dose level is about 0.5 mg/week or about 0.75 mg/week lower than the modified dose level if the IGF-1 level in the subject has an SDS of >+1.5 or wherein the further modified dose level is about 1.0 mg/week or about 1.5 mg/week higher than the modified dose level if the IGF-1 level in the subject has an SDS of <−0.5.

In some embodiments of the disclosure, a method of treating growth hormone deficiency in an adult subject in need thereof comprises i) administering a long-acting recombinant human growth hormone (rhGH) comprising the amino acid sequence of SEQ ID NO:2 to the subject at an initial dose level; ii) monitoring the subject for an adverse event; iii) administering the long-acting rhGH to the subject at a modified dose level wherein the modified dose level is 25% lower than the initial dose level if the adverse event is moderate, or wherein the modified dose level is 50% lower than the initial dose level if the adverse event is severe.

In some embodiments, the present teachings provide methods of treating growth hormone deficiency in a first subject in need thereof, the method comprising: selecting a first subject with growth hormone deficiency, wherein the first subject has previously received a once daily recombinant human growth hormone (once daily rhGH) therapy; and administering an effective amount of a long-acting recombinant human growth hormone (long-acting rhGH) to the first subject, so that efficacy of the long-acting rhGH in the first subject is comparable to efficacy of the long-acting rhGH in a second subject who has previously received only the long-acting rhGH and has not previously received the once daily rhGH therapy. A once daily rhGH is somatropin, somatrem, a somatropin biosimilar, or a somatrem biosimilar.

In some embodiments, provided herein is a use of a long-acting recombinant human growth hormone (long-acting rhGH) for treating growth hormone deficiency in a first subject in need thereof, the use comprising: selecting a first subject with growth hormone deficiency, wherein the first subject has previously received a once daily recombinant human growth hormone (once daily rhGH) therapy; and administering an effective amount of the long-acting rhGH to the first subject, so that efficacy of the long-acting rhGH in the first subject is comparable to efficacy of the long-acting rhGH in a second subject who has previously received only the long-acting rhGH and has not previously received the once daily rhGH therapy.

In some embodiments, provided herein is use of a long-acting recombinant human growth hormone (long-acting rhGH) for treating growth hormone deficiency in a subject in need thereof, the use comprising: selecting a subject with growth hormone deficiency, wherein the subject has previously received a once daily recombinant human growth hormone (once daily rhGH) therapy; and administering an effective amount of a long-acting recombinant human growth hormone (long-acting rhGH) to the subject once weekly, wherein a bone maturation rate of the subject previously on the once daily recombinant human growth hormone is comparable to a bone maturation rate of the subject while on the once daily recombinant treatment.

In some embodiments, provided herein is a method of treating growth hormone deficiency in a subject in need thereof, the method comprising: selecting a subject with growth hormone deficiency, wherein the subject has previously received a once daily recombinant human growth hormone (once daily rhGH) therapy; and administering an effective amount of a long-acting recombinant human growth hormone (long-acting rhGH) to the subject once weekly, wherein a bone maturation rate of the subject previously on the once daily recombinant human growth hormone is comparable to a bone maturation rate of the subject while on the once daily recombinant treatment.

Other features and advantages will become apparent from the following detailed description examples and figures. Exemplary methods and materials are described herein, although methods and materials similar or equivalent to those described herein can also be used in the practice or testing of the present invention. The materials, methods, and examples are illustrative only and not intended to be limiting.

### BRIEF DESCRIPTION OF THE DRAWINGS

The following drawings form part of the present specification and are included to further demonstrate certain embodiments of the present disclosure, the inventions of which can be better understood by reference to one or more of these drawings in combination with the detailed description of specific embodiments presented herein.

FIG. 1 depicts the study design for the open-label extension of a clinical trial study of once weekly somatrogon vs. daily Genotropin® in pediatric patients with growth hormone deficiency. OLE=open-label extension; PEN=somatrogon delivery via prefilled pen device; R=randomization.

FIG. 2 depicts a bar graph summarizing height SDS and cumulative delta height SDS for each year of the study (all cohorts combined). X-axis: Mean cumulative delta height SDS±SD and Mean Height SDS±SD. Dark bars below: Mean height SDS±SD. Lighter bars above: Mean cumulative delta height SDS±SD. OLE=open-label extension; SD=standard deviation; SDS=standard deviation score; Y=year.

FIG. 3 depicts a bar graph summarizing data from the QoLISSY-CHILD survey: mean change from BL to 12 months, children aged≥7 years. BL=baseline; QoL=quality of life; QoLISSY=Quality of Life in Short Stature Youth.

FIG. 4 depicts a bar graph summarizing data from the QoLISSY-PARENT survey: mean change from BL to 12 months, children aged≥7 years. BL=baseline; QoL=quality of life; QoLISSY=Quality of Life in Short Stature Youth.

FIG. 5 depicts a bar graph summarizing data from the QoLISSY-CHILD survey: BL (B) and 12 month (M12) scores, children aged≥7 years. QoL=quality of life; QoLISSY=Quality of Life in Short Stature Youth.

FIG. 6 depicts a bar graph summarizing data from the QoLISSY-PARENT survey: BL (B) and 12 month (M12) scores, children aged≥7 years. QoL=quality of life; QoLISSY=Quality of Life in Short Stature Youth.

FIG. 7 depicts a schematic summarizing overall life interference total scores (DCOA 1). Box shows interquartile range (IQR); whiskers include observed values within 1.5×IQR from the box edges. ^{a} Number of participants with non-missing values.

FIG. 8 depicts a diagram summarizing data from the patient and caregiver assessment of treatment experience (DCOA 1).

FIG. 9 depicts a diagram summarizing data from the DCOA 2 questionnaire. * “Does not favor somatrogon” includes Genotropin® and No preference/No difference.

FIG. 10 depicts a box plot of height velocity over time (full analysis set). Baseline defined as the last non-missing measurement prior to the start of study drug. Somatrogon data is indicated by a circle. Genotropin® data is indicated by a square.

FIG. 11 depicts a box plot of IGF-1 SDS over time. Somatrogon data is indicated by a circle. Genotropin® data is indicated by a square.

FIG. 12 depicts graphs summarizing deconvoluted, zero-change mass spectral data of intact somatrogon from Process C-Rentschler Biotpharma (PRC-RB) and Process C-Grange Castle (PRC-GC) materials.

FIG. 13 depicts a Phase 3 study design (top panel) and subject disposition in a Phase 3 study (bottom panel).

FIG. 14 depicts subgroup analyses for the primary endpoint of height velocity at month 12. Region 1 includes Western Europe, Israel, Greece, Australia, New Zealand, Canada, and USA. Region 2 includes Central and Eastern Europe, Turkey, Latin America and Asia except for India and Vietnam. Region 3 includes India and Vietnam. ^{a} Number of participants with non-missing values.

FIG. 15 depicts a summary of height velocity (cm/year) (top panel) and height SDS over time. Somatrogon data is indicated by a circle. Genotropin® data is indicated by a square.

FIG. 16 depicts IGF-1 SDS over time. Somatrogon data is indicated by a circle. Genotropin® data is indicated by a square.

### DETAILED DESCRIPTION

Provided herein are uses, methods, and therapeutic regimens for treatment of growth deficiency disorders. The subject use, methods, and therapeutic regimens involve administration of a long-acting recombinant human growth hormone (rhGH). The subject use and therapeutic regimens can be used in the treatment of growth deficiency disorders.

### Definitions

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference. Other general references are provided throughout this document.

It is understood that wherever embodiments are described herein with the language “comprising,” otherwise analogous embodiments described in terms of “consisting of” and/or “consisting essentially of” are also provided. Where embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

As used herein, the term “about” refers to a ±10% numerical range of a given value. For example, a dosage of about 50 milligrams per kilogram (mg/kg) refers to a range of 45 to 55 mg/kg.

In some embodiments, “active ingredient” refers to a polypeptide sequence of interest, which is accountable for the biological effect. In some embodiments, an active ingredient is a long-acting rhGH. In some embodiments, an active ingredient is somatrogon, that is, a polypeptide comprising the amino acid sequence of SEQ ID NO:2. In some embodiments, an active ingredient is a once daily rhGH, e.g., Genotropin®.

As used herein, the term “between” refers to a numerical range that is inclusive of the two endpoint values of the numerical range. For example, a range that is “between 12 to 18” is inclusive of the endpoint values 12 and 18.

As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the term “standard derivation score” (SDS) quantifies a measurement's variation from an average or mean value. A lower standard deviation score means that the measurement is closer to the average or mean, while a high standard deviation score means that the value is further from the average or mean. In some embodiments, SDS is calculated using the modified least squares (LS) mean model (Bidlingmaier et al., J. Clin. Endocrinol. Metab. (2014) 99(5):1712-1721). In some embodiments, estimated SDS profiles over a dosing interval is calculated according to Fisher et al., Horm. Res. Paediatr. (2017) 87(5):324-332.

As used herein, the term “dosing regimen” refers to a total course of treatment administered to a patient, e.g., treatment with a long-acting rhGH. In some embodiments, a dosing regimen comprises administering a long-acting rhGH on a weekly basis. In some embodiments, a dosing regimen is every 3 days, every 4 days, every 5 days, every 6 days, every 7 days, or every 9 days. In some embodiments, a dosing regimen comprises administering a long-acting rhGH every 3 to 5 days, every 4 to 6 days, every 5 to 7 days, or every 6 to 8 days.

In some embodiments, a dosing regimen includes a dose modification regimen for a long-acting rhGH, optionally in response to a serum IGF-1 level with a standard deviation score (SDS) of >+2 or <−2 (which may be abbreviated as >+2 SDS or <−2 SDS).

In some embodiments, a dosing regimen includes a dose reduction regimen for a long-acting rhGH, optionally in response to a serum IGF-1 level>+2 standard deviation score (SDS). In some embodiments, a dosing regimen includes a dose reduction regimen for a long-acting rhGH, optionally in response to a serum IGF-1 level>+2 standard deviation score (SDS) wherein an initial dose level (e.g., about 0.66 mg/kg/week) is reduced by about 5% to about 50% (e.g., about 15%, about 30%) to a modified dose level or a further modified dose level. In some embodiments, a dose reduction regimen may also be referred to a dose modification regimen.

In some embodiments, a dosing regimen includes a dose increase regimen for a long-acting rhGH, optionally in response to a serum IGF-1 level<−2 standard deviation score (SDS). In some embodiments, a dosing regimen includes a dose increase regimen for a long-acting rhGH, optionally in response to a serum IGF-1 level<−2 standard deviation score (SDS), wherein an initial dose level (e.g., about 0.66 mg/kg/week) is increased by about 5% to about 50% (e.g., about 15%, about 30%) to a modified dose level or a further modified dose level. In some embodiments, a dose increase regimen may also be referred to a dose modification regimen.

In some embodiments, a dosing regimen includes a dose modification regimen for a long-acting rhGH, optionally in response to a serum IGF-1 level having a standard deviation score of >+1.5 or <−0.5. In some embodiments, a dosing regimen includes a dose modification regimen for a long-acting rhGH, optionally in response to a serum IGF-1 level having a standard deviation score of >+2 or <−2.

In some embodiments, a dosing regimen includes a dose reduction regimen for a long-acting rhGH, optionally in response to a serum IGF-1 level>+1.5 standard deviation score (SDS). In some embodiments, a dosing regimen includes a dose reduction regimen for a long-acting rhGH, optionally in response to a serum IGF-1 level>+1.5 standard deviation score (SDS) wherein an initial dose level (e.g., 1-5 mg/week) is reduced by about 0.1 mg/week to about 1.0 mg/week (e.g., about 0.5 mg/week, about 0.75 mg/week) to a modified dose level or a further modified dose level.

In some embodiments, a dosing regimen includes a dose increase regimen for a long-acting rhGH, optionally in response to a serum IGF-1 level<−0.5 standard deviation score (SDS). In some embodiments, a dosing regimen includes a dose increase regimen for a long-acting rhGH, optionally in response to a serum IGF-1 level<−0.5 standard deviation score (SDS), wherein an initial dose level (e.g., 1-5 mg/week) is increased by about 0.5 mg/week to about 2 mg/week (e.g., about 1 mg/week, about 1.5 mg/week) to a modified dose level or a further modified dose level.

As used herein, “efficacy” refers to the capacity of a drug or treatment to produce a pharmacological effect.

In some embodiments, efficacy is assessed by measuring one or more of: mean height velocity, gain (delta) in height standard deviation score (SDS), body mass index, bone maturation, insulin growth factor-1 (IGF-1) SDS, insulin-like growth factor binding protein 3 IGFBP-3 SDS, pubertal status changed from Tanner 1, mean glucose, HbA1c, thyroid function, and cholesterol values. Height SDS is derived from the age and sex standards from the 2000 Centers for Disease Control Growth Charts (Centers for Disease Control. Growth Charts. 2010 (last update Sep. 9, 2010) at www[dot]cdc[dot]gov/growthcharts/).

In some embodiments, efficacy is indicated by continued bone maturation.

In some embodiments, efficacy is assessed by the Quality of Life in Short Stature Youth (QoLISSY) questionnaire, which assesses the impact of short stature on the QoL in children, during the first 12 months of treatment. The response scale is a 5-point Likert scale (“not at all/never” to “extremely/always”). Scores>70 indicate a good QoL. See Table 5.

In some embodiments, efficacy is assessed by the Dyad Clinical Outcome Assessment (DCOA) questionnaire. The DCOA questionnaire is completed as a Dyad pair (child and caregiver together), with some specific questions intended for the caregiver only. The DCOA questionnaire is comprised of 2 parts (DCOA 1 and 2), with a comprehensive list of questions to determine the treatment burden. Patients and caregivers rate treatment experience as part of DCOA 1, and select their preference for either daily or weekly injections as part of DCOA 2.

As used herein, an “effective dosage,” “effective amount,” “therapeutically effective amount” or “therapeutically effective dosage” of drug, compound, or pharmaceutical composition is an amount sufficient to affect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results, decreasing the dose of other medications required to treat the disease, enhancing the effect of another medication, and/or delaying the progression of the disease of patients.

In some embodiments, an effective amount of a long-acting rhGH (e.g., somatrogon) maintains a serum or plasma IGF-1 level in a subject within +/−2 SDS. In some embodiments, an effective amount of a long-acting rhGH, increases a serum or plasma IGF-1 level in a subject to within +/−2 SDS. In some embodiments, an effective amount of a long-acting rhGH (e.g., somatrogon) increases and maintains a serum or plasma IGF-1 level in a subject within +/−2 SDS.

In some embodiments, an effective amount of a long-acting rhGH (e.g., somatrogon) maintains a serum or plasma IGF-1 level in a subject within +/−1.5 SDS. In some embodiments, an effective amount of a long-acting rhGH, increases a serum or plasma IGF-1 level in a subject to within +/−1.5 SDS. In some embodiments, an effective amount of a long-acting rhGH (e.g., somatrogon) increases and maintains a serum or plasma IGF-1 level in a subject within +/−1.5 SDS.

In some embodiments, an effective amount of a long-acting rhGH (e.g., somatrogon) maintains a serum or plasma IGF-1 level in a subject between a standard deviation score of −0.5 and +1.5. In some embodiments, an effective amount of a long-acting rhGH, increases a serum or plasma IGF-1 level in a subject to between a standard deviation score of −0.5 and +1.5. In some embodiments, an effective amount of a long-acting rhGH (e.g., somatrogon) increases and maintains a serum or plasma IGF-1 level in a subject between a standard deviation score of −0.5 and +1.5.

In some embodiments, IGF-1 SDS is calculated using the modified least squares (LS) mean model (Bidlingmaier et al., J. Clin. Endocrinol. Metab. (2014) 99(5):1712-1721). In some embodiments, estimated IGF-1 SDS profiles over the dosing interval is calculated according to Fisher et al., Horm. Res. Paediatr. (2017) 87(5):324-332.

In some embodiments, an effective amount of a long-acting rhGH (e.g., somatrogon) decreases trunk fat mass, increases lean body mass, decreases trunk fat mass as a percentage of total fat mass, normalizes IGF-1 levels or a combination thereof in a subject (e.g., an adult).

An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

In some embodiments, an effective amount of a long-acting rhGH is administered based on the weight of a subject, for instance mg per kg of body weight. In some embodiments, an effective amount of a long-acting rhGH is administered based on the weight of a subject and on a dose interval, for instance, mg per kg of body weight per week. In some embodiments, an effective amount of a long-acting rhGH is adjusted every 1 to 6 months (e.g., every 1, every 2, every 3, every 4, every 5, or every 6 months) based on a subject's body weight.

In some embodiments, an effective amount of a long-acting rhGH is about 0.66 mg/kg body weight/week. In some embodiments, an effective amount of a long-acting rhGH is about 0.56 mg/kg body weight/week. In some embodiments, an effective amount of a long-acting rhGH is about 0.48 mg/kg body weight/week. In some embodiments, an effective amount of a long-acting rhGH is about 0.40 mg/kg body weight/week. In some embodiments, an effective amount of a long-acting rhGH is about 0.36 mg/kg body weight/week. In some embodiments, an effective amount of a long-acting rhGH, is about 0.25 mg/kg body weight/week. In some embodiments, an effective amount of a long-acting rhGH is about 0.16 mg/kg body weight/week. In some embodiments, an effective amount of a long-acting rhGH is about 0.16 mg/kg body weight/week to about 0.66 mg/kg body weight/week. In some embodiments, an effective amount of a once daily rhGH therapy is about 0.10 mg to about 1.0 mg per kg body weight per week.

In some embodiments, an effective amount of a long-acting rhGHis administered as a fixed dose at a particular interval, for instance, mg per week (e.g., 6 to 8 days). In some embodiments, an effective amount of a long-acting rhGH is administered based on the gender, age and/or estrogen status of a subject. In some embodiments, an effective amount of a long-acting rhGH is adjusted based on a subject's age and/or estrogen status.

In some embodiments, an effective amount of a long-acting rhGH is about 0.66 mg/week, about 0.56 mg/week, about 0.48 mg/week, about 0.40 mg/week, about 0.36 mg/week, about 0.25 mg/week, or about 0.16 mg/week for a pediatric subject. In some embodiments, an effective amount of a long-acting rhGH is between 0.16 mg/week to 0.66 mg/week for a pediatric subject.

In some embodiments, an effective amount of a long-acting rhGH is about 2.0 mg/week, about 2.5 mg/week, or about 3.5 mg/week for a male 50 years of age or less.

In some embodiments, an effective amount of a long-acting rhGH is about 1.5 mg/week, about 2.0 mg/week, or about 3.5 mg/week for a male greater than 50 years of age.

In some embodiments, an effective amount of a long-acting rhGH is about 2.5 mg/week, about 3.0 mg/week, or about 4.0 mg/week for a female not on oral estrogen who is 50 years of age or less.

In some embodiments, an effective amount of a long-acting rhGH is about 2.0 mg/week, about 2.5 mg/week, or about 3.5 mg/week for a female not on oral estrogen who is greater than 50 years of age.

In some embodiments, an effective amount of a long-acting rhGH is about 3.25 mg/week, about 4.0 mg/week, or about 5.5 mg/week for a female on oral estrogen who is 50 years of age or less.

In some embodiments, an effective amount of a long-acting rhGH is about 2.75 mg/week, about or 3.5 mg/week, or about 5.0 mg/week for a female on oral estrogen who is greater than 50 years of age.

In some embodiments, an effective amount of a long-acting rhGH ranges from about 1 mg/week to about 11 mg/week for an adult with growth hormone deficiency. In some embodiments, an effective amount of a long-acting rhGH ranges from about 1 mg/week to about mg/week for an adult with growth hormone deficiency.

As used herein, “GH” refers to growth hormone from any species, including bovine, ovine, porcine, equine, and preferably human, in native-sequence or invariant form, and from any source, whether natural, synthetic, or recombinant. In some embodiments, the phrase “human growth hormone” (hGH) refers to a polypeptide, such as set forth in Genbank Accession No. P01241 (SEQ ID NO:1). The hGH sequence shown in SEQ ID NO:1 is further processed to remove the first 26 N-terminal amino acids corresponding to a signal peptide (underlined), resulting in the mature, 191 amino acid form exhibiting hGH activity (i.e. stimulation of growth).

(SEQ ID NO: 1)
MATGSRTSLLLAFGLLCLPWLQEGSAFPTIPLSRLFDNAMLRAHRLHQLA
FDTYQEFEEAYIPKEQKYSFLQNPQTSLCFSESIPTPSNREETQQKSNLE
LLRISLLLIQSWLEPVQFLRSVFANSLVYGASDSNVYDLLKDLEEGIQTL
MGRLEDGSPRTGQIFKQTYSKFDTNSHNDDALLKNYGLLYCFRKDMDKVE
TFLRIVQCRSVEGSCGF

In other embodiments, “GH” also refers to homologues. In other embodiments, a GH amino acid sequence of the methods and compositions the present invention is at least 50% homologous to a GH sequence set forth herein as determined using BlastP software of the National Center of Biotechnology Information (NCBI) using default parameters. In other embodiments, a percent homology is 60%. In other embodiments, a percent homology is 70%. In other embodiments, a percent homology is 80%. In other embodiments, a percent homology is 90%. In other embodiments, a percent homology is at least 95%. In other embodiments, a percent homology is greater than 95%. Each possibility represents a separate embodiment of the present invention. As used herein, the term “homology” encompasses deletions, insertions, or substitution variants, including an amino acid substitution thereof, and biologically active polypeptide fragments thereof.

As used herein, “IGF-1” refers to insulin-like growth factor from any species, including bovine, ovine, porcine, equine, and preferably human, in native-sequence or in variant form, and from any source, whether natural, synthetic, or recombinant. IGF-1 is a secreted from the liver and other tissues in response to growth hormone. In some embodiments, IGF-1 is a validated surrogate marker for hGH activity. In some embodiments, a serum or plasma level of IGF-1 in a subject is increased following administration of a long-acting rhGH (e.g., somatrogon). In some embodiments, a serum or plasma level of IGF-1 in a subject is maintained following administration of a long-acting rhGH (e.g., somatrogon). In some embodiments, a serum or plasma level of IGF-1 in a subject is increased and maintained following administration of a long-acting rhGH (e.g., somatrogon). In some embodiments, a serum or plasma level of IGF-1 in a subject is maintained within a defined range following administration of a long-acting rhGH (e.g., somatrogon). In some embodiments, a defined range of a serum or plasma IGF-1 level is comparable to a range of serum or plasma IGF-1 levels in individuals without growth hormone deficiency. In some embodiments, a defined range of a serum or plasma IGF-1 level is the range of serum or plasma IGF-1 levels in a control population.

In some embodiments, a desired therapeutic range of IGF-1 in a subject treated with a long-acting rhGH (e.g., somatrogon) is defined as a range between +2 standard deviations through −2 standard deviations from the average IGF-1 levels expected in an appropriate control population, where the control population is a reference population stratified by age group and gender. In some embodiments, a desired therapeutic range of IGF-1 in a subject treated with a long-acting rhGH (e.g., somatrogon) is defined as a range between +1.5 standard deviations through −1.5 standard deviations from the average IGF-1 levels expected in a control population, stratified by age group and gender. In some embodiments, a desired therapeutic range of IGF-1 in a subject treated with a long-acting rhGH (e.g., somatrogon) is defined as a range between +1.5 standard deviations through −0.5 standard deviations from the average IGF-1 levels expected in a control population, stratified by age group and gender.

In some embodiments, IGF-1 standard deviation score (SDS) is calculated using the modified least squares (LS) mean model (Bidlingmaier et al., J. Clin. Endocrinol. Metab. (2014) 99(5):1712-1721). In some embodiments, estimated IGF-1 SDS profiles over the dosing interval is calculated according to Fisher et al., Horm. Res. Paediatr. (2017) 87(5):324-332.

As used herein, the term “subject” refers to a mammal, more preferably, a human. Mammals also include, but are not limited to, sport animals, pets, primates, horses, dogs, cats, mice, rats, and farm animals including without limitation cows, pigs, goats, and sheep. In some embodiments, a subject is a human with growth hormone deficiency. In some embodiments, a subject with growth hormone deficiency has impaired height (SDS≤−2) and impaired height velocity (HV) (e.g., annualized HV below the 25^{th} percentile for chronological age [HV<−0.7 SDS]), an IGF-1 level≥1 SD below the age- and sex-standardized mean IGF-1 level (e.g., SDS≤−1) and/or has not received prior rhGH therapy. In some embodiments, a subject with growth hormone deficiency is a pediatric subject, for example a subject up to the age of 18 year. In some embodiments, a subject with growth hormone deficiency is a female of 3 to ≤10 years of age. In some embodiments, a subject with growth hormone deficiency is a male of 3 to ≤11 years of age.

In some embodiments, a subject with growth hormone deficiency has: impaired height (standard deviation score (SDS)≤−2), impaired height velocity below the 25^{th} percentile for chronological age, an IGF-1 level at least 1 SD below the age and sex-standardized mean IGF-1 level (SDS≤−1), and/or has not received prior rhGH therapy.

In some embodiments, a subject with an SDS<−2 is a subject whose serum IGF-1 concentration is below the mean reference value for their age and sex by more than 2 SDS. In some embodiments, IGF-1 SDS is calculated using the modified least squares (LS) mean model (Bidlingmaier et al., J. Clin. Endocrinol. Metab. (2014) 99(5):1712-1721). In some embodiments, estimated IGF-1 SDS profiles over the dosing interval is calculated according to Fisher et al., Horm. Res. Paediatr. (2017) 87(5):324-332.

In some embodiments, a subject does not have active malignancy, a prior history of a malignancy or received radiation therapy or chemotherapy. In some embodiments, a subject does not have an acute illness such as for example, complications following open heart or abdominal surgery, multiple accidental trauma, or acute respiratory failure. In some embodiments, a subject does not have a body mass index (BMI)<−2 SDS (age- and sex-standardized), anti-rhGH antibodies at screening, psychosocial dwarfism, a chromosomal abnormality (e.g., Turner syndrome, Laron syndrome, Noonan syndrome, Prader-Willi syndrome, Russell-Silver syndrome, SHOX mutations/deletions, or skeletal dysplasia), celiac disease, uncontrolled primary hypothyroidism, rickets or who was born small for their gestational age (birth weight/length<−2 SDS). In some embodiments, a subject does not have type 1 or type 2 diabetes mellitus and is not receiving standard of care, is noncompliant with their prescribed treatment, or is in poor metabolic control. In some embodiments, a subject is not receiving anabolic/sex steroid (except for drugs for ADHD or hormone replacement therapies), glucocorticoid therapy or inhaled budesonide at dose greater than 400 μg/day or equivalent. In some embodiments, a subject does not have >1 closed epiphyses, is not HIV-positive or with advanced diseases such as AIDS or tuberculosis, is not hypersensitive to components of study medication.

In some embodiments, a subject had received a once daily recombinant human growth hormone for at least three, four, five, six, seven, eight, nine, ten, eleven months or twelve months. In some embodiments, a subject had received a once daily recombinant human growth hormone for at least one year. In some embodiments, a subject had not received a once daily recombinant human growth hormone.

In some embodiments, a subject is obese. In some embodiments, a subject is female. In some embodiments, the subject is 10 to 15 years old. In some embodiments, a subject is 3 to ≤11 years old, and optionally a male. In some embodiments, a subject is 3 to ≤10 years of age, and optionally a female.

In some embodiments, a subject is a pediatric subject. In some embodiments, a pediatric subject is three years old or older.

In some embodiments, a subject has one or more of the following: isolated growth hormone deficiency (GHD), GH insufficiency as part of multiple pituitary hormone deficiency, pediatric GHD, and Prader-Willi Syndrome. In some embodiments, a subject has adult.

As used herein, the terms “treat,” or “treatment” is an approach for obtaining beneficial or desired clinical results. In some embodiments, the terms “treat” or “treatment” means to administer a therapy that partially or completely alleviates, ameliorates, relieves, inhibits, delays onset of, reduces severity of, and/or reduces incidence of one or more symptoms, features and causes of a particular disease, disorder and/or condition (e.g., growth hormone deficiency). For purposes of this invention, beneficial or desired clinical results include, but are not limited to, one or more of the following: improved height velocity, bone maturation, IGF-1 level related to growth hormone deficiency. The term includes the administration of a compound or agent of the present invention to prevent or delay the onset of a symptom, complication, or biochemical indicia of a disease, alleviating a symptom or arresting or inhibiting further development of a disease, condition, or disorder. Treatment may be prophylactic (to prevent or delay the onset of the disease, or to prevent the manifestation of a clinical or subclinical symptom thereof) or therapeutic suppression or alleviation of a symptom after the manifestation of the disease. In some embodiments, the disease, condition or disorder is growth hormone deficiency.

In some embodiments, a “pharmaceutical composition” refers to a preparation of one or more active ingredients described herein with other chemical components such as physiologically suitable carriers and excipients. The purpose of a pharmaceutical composition is to facilitate administration of a compound to an organism.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

### Long Acting Recombinant Human Growth Hormone

In some embodiments, the present teachings provide long-acting recombinant human growth hormone (long-acting rhGH) and methods of producing and using the same. The long-acting rhGH provided herein comprises recombinant human growth hormone (rhGH) and carboxy terminal peptides (CTPs) of human chorionic gonadotropin (hCG). In some embodiments, CTP acts as a protectant against degradation of proteins or peptides derived therefrom. In other embodiments, CTP extends circulatory half-lives of proteins or peptides derived therefrom. In some embodiments, CTP enhances the potency of proteins or peptides derived therefrom. In some embodiments, the long-acting rhGH is a CTP-modified growth hormone polypeptide as described in at least U.S. Pat. No. 7,553,941, granted Jun. 30, 2009; U.S. Pat. No. 8,097,435, granted Jan. 17, 2012; U.S. Pat. No. 8,048,849, granted Nov. 1, 2011; U.S. Pat. No. 8,450,269, granted May 28, 2013; U.S. Pat. No. 8,304,386, granted Nov. 6, 2012; U.S. Pat. No. 8,946,155, granted Feb. 3, 2015; U.S. Pat. No. 9,896,494, granted Feb. 20, 2018; U.S. Pat. No. 8,450,269, granted May 28, 2013; U.S. Pat. No. 10,351,615, granted Jul. 16, 2019; U.S. Pat. No. 11,197,915, granted Dec. 14, 2021; and PCT Application Publication Nos. WO2007094985, WO2013018098, WO2014080401, WO2015059695, WO2016092550 and WO2016092549, each of which is incorporated herein by reference in its entirety for all of its materials, methods, and teachings.

The terms “CTP peptide,” “carboxy terminal peptide,” “CTP sequence,” and “chorionic gonadotropin C-terminal peptide” are used interchangeably herein. In other embodiments, the carboxy terminal peptide is a full-length CTP. In other embodiments, the carboxy terminal peptide is a truncated CTP. Each possibility represents a separate embodiment of the present invention. In some embodiments, a CTP peptide comprises an amino acid sequence of SEQ ID NO:3 (SSSSKAPPPSLPSPSRLPGPSDTPILPQ).

In some embodiments, a long-acting rhGH is a C-terminal peptide (CTP)-modified hGH. In some embodiments, a long-acting rhGH comprises the amino acid sequence of mature human growth hormone (hGH) with one copy of CTP from the beta chain of human chorionic gonadotropin at the hGH N-terminus and two copies of CTP in tandem at the hGH C-terminus. In some embodiments, a long-acting rhGH comprises the amino acid sequence shown in SEQ ID NO: 2. In some embodiments, a long-acting rhGH is glycosylated. In some embodiments, a long-acting rhGH is O-glycosylated on twelve to eighteen serines. In some embodiments, a long-acting rhGH is O-glycosylated on twelve to twenty serines. In some embodiments, a long-acting rhGH is O-glycosylated on ten to twenty serines.

In some embodiments, the present teachings provide pharmaceutical formulations comprising a buffer, a tonicity agent, and a long-acting rhGH comprising a human growth hormone and one chorionic gonadotropin CTP attached to the amino terminus of the human growth hormone, and two chorionic gonadotropin CTPs attached to the carboxy terminus of the human growth hormone. In some embodiments, the long-acting rhGH is somatrogon. Somatrogon is a long-acting rhGH comprising the amino acid sequence of human growth hormone and three copies of the carboxy-terminal peptide of human chorionic gonadotropin. In some embodiments, the long-acting rhGH comprises the amino acid sequence of SEQ ID NO: 2. SSSSKAPPPSLPSPSRLPGPSDTPILPQFPTIPLSRLFDNAMLRAHRLHQLAFDTYQEFEEA YIPKEQKYSFLQNPQTSLCFSESIPTPSNREETQQKSNLELLRISLLLIQSWLEPVQFLRSVF ANSLVYGASDSNVYDLLKDLEEGIQTLMGRLEDGSPRTGQIFKQTYSKFDTNSHNDDAL LKNYGLLYCFRKDMDKVETFLRIVQCRSVEGSCGFSSSSKAPPPSLPSPSRLPGPSDTPIL PQSSSSKAPPPSLPSPSRLPGPSDTPILPQ (SEQ ID NO: 2). In some embodiments, a long-acting recombinant human growth hormone (rhGH) comprising the amino acid sequence of SEQ ID NO:2 further comprises O-glycans occupancy at between 9 to 20 and has at least 50% of the population of somatrogon molecules comprising between 12 to 18 O-glycans. In some embodiments, a long-acting recombinant human growth hormone (rhGH) comprising the amino acid sequence of SEQ ID NO:2 further comprises O-glycans occupancy at between 9 to 20 and has at least 60% of the population of somatrogon molecules comprising between 12 to 18 O-glycans. In some embodiments, a long-acting recombinant human growth hormone (rhGH) comprising the amino acid sequence of SEQ ID NO:2 further comprises O-glycans occupancy at between 9 to 20 and has at least 70% of the population of somatrogon molecules comprising between 12 to 18 O-glycans. In some embodiments, a long-acting recombinant human growth hormone (rhGH) comprising the amino acid sequence of SEQ ID NO:2 further comprises 0-glycans occupancy at between 9 to 20 and has at least 80% of the population of somatrogon molecules comprising between 12 to 18 O-glycans.

In some embodiments, a long-acting rhGH is somatrogon. Somatrogon is a glycoprotein comprised of the amino acid sequence of human growth hormone (hGH) with one copy of the C-terminal peptide (CTP) from the beta chain of human chorionic gonadotropin (hCG) at the N-terminus and two copies of CTP (in tandem) at the C-terminus. Each CTP includes multiple O-linked glycosylation sites. The glycosylation and CTP domains account for the half-life of somatrogon which allows for weekly dosing. The O-glycan occupancy ranges from 9 to 20 moieties per intact somatrogon molecule. The predominant somatrogon glycoforms include the molecule with 15 monosialylated, core-1 O-glycans or 16 monosialylated, core-1 O-glycans. Additionally, each CTP region contains hydroxyproline residues, which range from 0-5 hydroxy additions per intact somatrogon molecule. The amino acid sequence of somatrogon is set forth in SEQ ID NO:2. Somatrogon comprises one disulfide bridge between cysteine residue 81 and cysteine residue 193 of SEQ ID NO:2 and a second disulfide bridge between cysteine residue 210 and cysteine residue 217 of SEQ ID NO:2. In some embodiments, the long-acting rhGH comprises a composition of somatrogon molecules wherein at least 50%, at least 60%, at least 70%, at least 80%, at least 90%, or at least 95% of a population of somatrogon molecules comprises between 12 to 18 O-glycans. In some embodiments, the long-acting rhGH comprises a composition of somatrogon molecules provided at a dose of 0.66 milligrams (mg) per kilogram (kg) of body weight per week wherein at least 50%, at least 60%, at least 70%, at least 80%, at least 90%, or at least 95% of a population of somatrogon molecules comprises between 10 to 18 O-glycans. In some embodiments, the long-acting rhGH comprises a composition of somatrogon molecules provided at a dose of 0.66 milligrams (mg) per kilogram (kg) of body weight per week wherein at least 50%, at least 60%, at least 70%, at least 80%, at least 90%, or at least 95% of a population of somatrogon molecules comprises between 12 to 18 O-glycans. In some embodiments, the long-acting rhGH comprises a composition of somatrogon molecules provided at a dose of 0.56 milligrams (mg) per kilogram (kg) of body weight per week wherein at least 50%, at least 60%, at least 70%, at least 80%, at least 90%, or at least 95% of a population of somatrogon molecules comprises between 10 to 18 O-glycans. In some embodiments, the long-acting rhGH comprises a composition of somatrogon molecules provided at a dose of 0.56 milligrams (mg) per kilogram (kg) of body weight per week wherein at least 50%, at least 60%, at least 70%, at least 80%, at least 90%, or at least 95% of a population of somatrogon molecules comprises between 12 to 18 O-glycans.

In some embodiments, the long-acting rhGH polypeptide is glycosylated. In some embodiments, each CTP present in SEQ ID NO: 2 includes multiple O-linked glycosylation sites. In some embodiments, O-glycan occupancy can range from 9 to 20 moieties per intact long-acting rhGH molecule. In some embodiments, O-glycan occupancy can range from 12 to 18 moieties per intact long-acting rhGH molecule. In some embodiments, at least 50% of a population of long-acting rhGH polypeptide molecules has an O-glycan occupancy of between 12 to 18 moieties per intact long-acting rhGH polypeptide molecule.

The present application further provides for, and includes, a long-acting glycosylated rhGH polypeptide having an O-glycan occupancy range from 9 to 20 moieties per intact long-acting rhGH molecule is provided at a dose level of 0.66 milligrams (mg) per kilogram (kg) of body weight per week, wherein at least 50% of the rhGH molecules have an O-glycan occupancy of between 10 to 18 moieties per intact long-acting rhGH polypeptide molecule. In another embodiment, the long-acting glycosylated rhGH polypeptide having an O-glycan occupancy range from 9 to 20 moieties per intact long-acting rhGH molecule is provided at a dose level of 0.66 milligrams (mg) per kilogram (kg) of body weight per week, wherein at least 60% of the rhGH molecules have an O-glycan occupancy of between 10 to 18 moieties per intact long-acting rhGH polypeptide molecule. In a further embodiment, the long-acting glycosylated rhGH polypeptide having an O-glycan occupancy range from 9 to 20 moieties per intact long-acting rhGH molecule is provided at a dose level of 0.66 milligrams (mg) per kilogram (kg) of body weight per week, wherein at least 70% of the rhGH molecules have an 0-glycan occupancy of between 10 to 18 moieties per intact long-acting rhGH polypeptide molecule. In an embodiment, the long-acting glycosylated rhGH polypeptide has an O-glycan occupancy range from 9 to 20 moieties per intact long-acting rhGH molecule and is provided at a dose level of 0.66 milligrams (mg) per kilogram (kg) of body weight per week, wherein at least 80% of the rhGH molecules have an O-glycan occupancy of between 10 to 18 moieties per intact long-acting rhGH polypeptide molecule. Also included are embodiments that provides for a long-acting glycosylated rhGH polypeptide having an O-glycan occupancy range from 9 to 20 moieties per intact long-acting rhGH molecule and is provided at a dose level of 0.66 milligrams (mg) per kilogram (kg) of body weight per week, wherein at least 90% of the rhGH molecules have an O-glycan occupancy of between 10 to 18 moieties per intact long-acting rhGH polypeptide molecule. In some embodiments, each CTP region can contain hydroxyproline residues, which can range from 0-5 hydroxy additions per intact long-acting rhGH molecule.

The present application further provides for, and includes, a long-acting glycosylated rhGH polypeptide having an O-glycan occupancy range from 9 to 20 moieties per intact long-acting rhGH molecule is provided at a dose level of 0.56 milligrams (mg) per kilogram (kg) of body weight per week, wherein at least 50% of the rhGH molecules have an O-glycan occupancy of between 10 to 18 moieties per intact long-acting rhGH polypeptide molecule. In another embodiment, the long-acting glycosylated rhGH polypeptide having an O-glycan occupancy range from 9 to 20 moieties per intact long-acting rhGH molecule is provided at a dose level of 0.56 milligrams (mg) per kilogram (kg) of body weight per week, wherein at least 60% of the rhGH molecules have an O-glycan occupancy of between 10 to 18 moieties per intact long-acting rhGH polypeptide molecule. In a further embodiment, the long-acting glycosylated rhGH polypeptide having an O-glycan occupancy range from 9 to 20 moieties per intact long-acting rhGH molecule is provided at a dose level of 0.56 milligrams (mg) per kilogram (kg) of body weight per week, wherein at least 70% of the rhGH molecules have an 0-glycan occupancy of between 10 to 18 moieties per intact long-acting rhGH polypeptide molecule. In an embodiment, the long-acting glycosylated rhGH polypeptide has an O-glycan occupancy range from 9 to 20 moieties per intact long-acting rhGH molecule and is provided at a dose level of 0.56 milligrams (mg) per kilogram (kg) of body weight per week, wherein at least 80% of the rhGH molecules have an O-glycan occupancy of between 10 to 18 moieties per intact long-acting rhGH polypeptide molecule. Also included are embodiments that provide for a long-acting glycosylated rhGH polypeptide having an O-glycan occupancy range from 9 to 20 moieties per intact long-acting rhGH molecule and provided at a dose level of 0.56 milligrams (mg) per kilogram (kg) of body weight per week, wherein at least 90% of the rhGH molecules have an O-glycan occupancy of between 10 to 18 moieties per intact long-acting rhGH polypeptide molecule. In some embodiments, each CTP region can contain hydroxyproline residues, which can range from zero to five hydroxy additions per intact long-acting rhGH molecule.

The present application further provides for, and includes, a long-acting glycosylated rhGH polypeptide having an O-glycan occupancy range from 9 to 20 moieties per intact long-acting rhGH molecule is provided at a dose level of 0.48 milligrams (mg) per kilogram (kg) of body weight per week, wherein at least 50% of the rhGH molecules have an O-glycan occupancy of between 10 to 18 moieties per intact long-acting rhGH polypeptide molecule. In another embodiment, the long-acting glycosylated rhGH polypeptide having an O-glycan occupancy range from 9 to 20 moieties per intact long-acting rhGH molecule is provided at a dose level of 0.48 milligrams (mg) per kilogram (kg) of body weight per week, wherein at least 60% of the rhGH molecules have an O-glycan occupancy of between 10 to 18 moieties per intact long-acting rhGH polypeptide molecule. In a further embodiment, the long-acting glycosylated rhGH polypeptide having an O-glycan occupancy range from 9 to 20 moieties per intact long-acting rhGH molecule is provided at a dose level of 0.48 milligrams (mg) per kilogram (kg) of body weight per week, wherein at least 70% of the rhGH molecules have an 0-glycan occupancy of between 10 to 18 moieties per intact long-acting rhGH polypeptide molecule. In an embodiment, the long-acting glycosylated rhGH polypeptide has an O-glycan occupancy range from 9 to 20 moieties per intact long-acting rhGH molecule and is provided at a dose level of 0.48 milligrams (mg) per kilogram (kg) of body weight per week, wherein at least 80% of the rhGH molecules have an O-glycan occupancy of between 10 to 18 moieties per intact long-acting rhGH polypeptide molecule. Also included are embodiments that provides for a long-acting glycosylated rhGH polypeptide having an O-glycan occupancy range from 9 to 20 moieties per intact long-acting rhGH molecule and provided at a dose level of 0.48 milligrams (mg) per kilogram (kg) of body weight per week, wherein at least 90% of the rhGH molecules have an O-glycan occupancy of between 10 to 18 moieties per intact long-acting rhGH polypeptide molecule. In some embodiments, each CTP region can contain hydroxyproline residues, which can range from zero to five hydroxy additions per intact long-acting rhGH molecule.

The present application further provides for, and includes, a long-acting glycosylated rhGH polypeptide having an O-glycan occupancy range from 9 to 20 moieties per intact long-acting rhGH molecule is provided at a dose level of 0.66 milligrams (mg) per kilogram (kg) of body weight per week, wherein at least 50% of the rhGH molecules have an O-glycan occupancy of between 12 to 18 moieties per intact long-acting rhGH polypeptide molecule. In another embodiment, the long-acting glycosylated rhGH polypeptide having an O-glycan occupancy range from 9 to 20 moieties per intact long-acting rhGH molecule is provided at a dose level of 0.66 milligrams (mg) per kilogram (kg) of body weight per week, wherein at least 60% of the rhGH molecules have an O-glycan occupancy of between 12 to 18 moieties per intact long-acting rhGH polypeptide molecule. In a further embodiment, the long-acting glycosylated rhGH polypeptide having an O-glycan occupancy range from 9 to 20 moieties per intact long-acting rhGH molecule is provided at a dose level of 0.66 milligrams (mg) per kilogram (kg) of body weight per week, wherein at least 70% of the rhGH molecules have an 0-glycan occupancy of between 12 to 18 moieties per intact long-acting rhGH polypeptide molecule. In an embodiment, the long-acting glycosylated rhGH polypeptide has an O-glycan occupancy range from 9 to 20 moieties per intact long-acting rhGH molecule and is provided at a dose level of 0.66 milligrams (mg) per kilogram (kg) of body weight per week, wherein at least 80% of the rhGH molecules have an O-glycan occupancy of between 12 to 18 moieties per intact long-acting rhGH polypeptide molecule. Also included are embodiments that provides for a long-acting glycosylated rhGH polypeptide having an O-glycan occupancy range from 9 to 20 moieties per intact long-acting rhGH molecule and is provided at a dose level of 0.66 milligrams (mg) per kilogram (kg) of body weight per week, wherein at least 90% of the rhGH molecules have an O-glycan occupancy of between 12 to 18 moieties per intact long-acting rhGH polypeptide molecule. In some embodiments, each CTP region can contain hydroxyproline residues, which can range from 0-5 hydroxy additions per intact long-acting rhGH molecule.

The present application further provides for, and includes, a long-acting glycosylated rhGH polypeptide having an O-glycan occupancy range from 9 to 20 moieties per intact long-acting rhGH molecule is provided at a dose level of 0.56 milligrams (mg) per kilogram (kg) of body weight per week, wherein at least 50% of the rhGH molecules have an O-glycan occupancy of between 12 to 18 moieties per intact long-acting rhGH polypeptide molecule. In another embodiment, the long-acting glycosylated rhGH polypeptide having an O-glycan occupancy range from 9 to 20 moieties per intact long-acting rhGH molecule is provided at a dose level of 0.56 milligrams (mg) per kilogram (kg) of body weight per week, wherein at least 60% of the rhGH molecules have an O-glycan occupancy of between 12 to 18 moieties per intact long-acting rhGH polypeptide molecule. In a further embodiment, the long-acting glycosylated rhGH polypeptide having an O-glycan occupancy range from 9 to 20 moieties per intact long-acting rhGH molecule is provided at a dose level of 0.56 milligrams (mg) per kilogram (kg) of body weight per week, wherein at least 70% of the rhGH molecules have an 0-glycan occupancy of between 12 to 18 moieties per intact long-acting rhGH polypeptide molecule. In an embodiment, the long-acting glycosylated rhGH polypeptide has an O-glycan occupancy range from 9 to 20 moieties per intact long-acting rhGH molecule and is provided at a dose level of 0.56 milligrams (mg) per kilogram (kg) of body weight per week, wherein at least 80% of the rhGH molecules have an O-glycan occupancy of between 12 to 18 moieties per intact long-acting rhGH polypeptide molecule. Also included are embodiments that provide for a long-acting glycosylated rhGH polypeptide having an O-glycan occupancy range from 9 to 20 moieties per intact long-acting rhGH molecule and provided at a dose level of 0.56 milligrams (mg) per kilogram (kg) of body weight per week, wherein at least 90% of the rhGH molecules have an O-glycan occupancy of between 12 to 18 moieties per intact long-acting rhGH polypeptide molecule. In some embodiments, each CTP region can contain hydroxyproline residues, which can range from zero to five hydroxy additions per intact long-acting rhGH molecule.

The present application further provides for, and includes, a long-acting glycosylated rhGH polypeptide having an O-glycan occupancy range from 9 to 20 moieties per intact long-acting rhGH molecule is provided at a dose level of 0.48 milligrams (mg) per kilogram (kg) of body weight per week, wherein at least 50% of the rhGH molecules have an O-glycan occupancy of between 12 to 18 moieties per intact long-acting rhGH polypeptide molecule. In another embodiment, the long-acting glycosylated rhGH polypeptide having an O-glycan occupancy range from 9 to 20 moieties per intact long-acting rhGH molecule is provided at a dose level of 0.48 milligrams (mg) per kilogram (kg) of body weight per week, wherein at least 60% of the rhGH molecules have an O-glycan occupancy of between 12 to 18 moieties per intact long-acting rhGH polypeptide molecule. In a further embodiment, the long-acting glycosylated rhGH polypeptide having an O-glycan occupancy range from 9 to 20 moieties per intact long-acting rhGH molecule is provided at a dose level of 0.48 milligrams (mg) per kilogram (kg) of body weight per week, wherein at least 70% of the rhGH molecules have an 0-glycan occupancy of between 12 to 18 moieties per intact long-acting rhGH polypeptide molecule. In an embodiment, the long-acting glycosylated rhGH polypeptide has an O-glycan occupancy range from 9 to 20 moieties per intact long-acting rhGH molecule and is provided at a dose level of 0.48 milligrams (mg) per kilogram (kg) of body weight per week, wherein at least 80% of the rhGH molecules have an O-glycan occupancy of between 12 to 18 moieties per intact long-acting rhGH polypeptide molecule. Also included are embodiments that provides for a long-acting glycosylated rhGH polypeptide having an O-glycan occupancy range from 9 to 20 moieties per intact long-acting rhGH molecule and provided at a dose level of 0.48 milligrams (mg) per kilogram (kg) of body weight per week, wherein at least 90% of the rhGH molecules have an O-glycan occupancy of between 12 to 18 moieties per intact long-acting rhGH polypeptide molecule. In some embodiments, each CTP region can contain hydroxyproline residues, which can range from zero to five hydroxy additions per intact long-acting rhGH molecule.

In some embodiments, the predominant glycoforms can include the long-acting rhGH molecule with 15 monosialylated, core-1 O-glycans or 16 monosialylated, core-1 O-glycans. Additionally, each CTP region can contain hydroxyproline residues, which can range from 0-5 hydroxy additions per intact long-acting rhGH molecule.

In some embodiments, the long-acting rhGH polypeptide comprises two disulfide bridges. In some embodiments of the long-acting rhGH comprising the amino acid sequence of SEQ ID NO: 2, one disulfide bridge is between cysteine residue 81 and cysteine residue 193 of SEQ ID NO: 2, and a second disulfide bridge is between cysteine residue 210 and cysteine residue 217 of SEQ ID NO: 2.

In some embodiments, a configuration of CTP-growth hormone-CTP-CTP as described herein comprises a growth hormone or an active fragment thereof connected via a linker to at least one CTP unit. In some embodiments, a linker is a peptide bond. In other embodiments, a configuration of CTP-growth hormone-CTP-CTP as described herein comprises a growth hormone or an active fragment thereof connected via a peptide bond to at least one CTP unit. In other embodiments, a CTP-growth hormone-CTP-CTP as described herein comprises a growth hormone or an active fragment thereof connected via a peptide bond to at least one CTP unit which is connected to an additional CTP unit via a peptide bond. In other embodiments, a polypeptide as described herein comprising a growth hormone fragment thereof and CTP units and/or fragments thereof are interconnected via a peptide bond. In other embodiments, one nucleic acid molecule encodes a polypeptide as described herein comprising a growth hormone and/or fragments thereof and CTP units and/or fragments thereof.

In other embodiments, a CTP is attached to the polypeptide sequence of interest via a linker. In other embodiments, at least one CTP is optionally attached to said polypeptide sequence of interest via a linker. In other embodiments, a linker which connects the CTP sequence to the polypeptide sequence of interest is a covalent bond. In other embodiments, a linker which connects a CTP sequence to a polypeptide sequence of interest is a peptide bond. In other embodiments, a linker which connects a CTP sequence to a polypeptide sequence of interest is a substituted peptide bond.

In some embodiments, a CTP sequence at the amino terminal end of a polypeptide, a CTP sequence at the carboxy terminal end of a polypeptide, and at least one additional CTP sequence attached in tandem to the CTP sequence at the carboxy terminus provide enhanced protection against degradation of a protein. In some embodiments, a CTP sequence at the amino terminal end of a polypeptide, a CTP sequence at the carboxy terminal end of the polypeptide, and at least one additional CTP sequence attached in tandem to the CTP sequence at the carboxy terminus provide an extended half-life to the attached protein. In some embodiments, a CTP sequence at the amino terminal end of a polypeptide, a CTP sequence at the carboxy terminal end of a polypeptide, and at least one additional CTP sequence attached in tandem to the CTP sequence at the carboxy terminus provide enhanced activity of the attached protein.

In other embodiments, at least one CTP sequence at the amino terminal end of a growth hormone and two CTP units in the carboxy terminal end of a growth hormone provide enhanced protection against clearance. In other embodiments, at least one CTP sequence at the amino terminal end of a growth hormone and two CTP units in the carboxy terminal end of a growth hormone provide prolonged clearance time. In other embodiments, at least one CTP sequence at the amino terminal end of a growth hormone and two CTP units in the carboxy terminal end of a growth hormone enhance C. of a growth hormone. In other embodiments, at least one CTP sequence at the amino terminal end of a growth hormone and two CTP units in the carboxy terminal end of a growth hormone enhance T. of a growth hormone. In other embodiments, at least one CTP sequence at the amino terminal end of a growth hormone and two CTP units in the carboxy terminal end of a growth hormone enhanced T_{1/2}.

In some embodiments, CTP sequences at both the amino terminal end of a growth hormone and at the carboxy terminal end of a growth hormone extend the half-life of the modified growth hormone. In other embodiments, at least a single CTP sequence at the amino terminal end of a growth hormone and at least two CTP sequences at the carboxy terminal end of a growth hormone provide an extended half-life to the modified growth hormone. In other embodiments, a single CTP sequence at the amino terminal end of a growth hormone and two CTP sequences at the carboxy terminal end of a growth hormone provide extended half-life to the attached growth hormone. In other embodiments, a single CTP sequence at the amino terminal end of a growth hormone and two CTP sequences in tandem at the carboxy terminal end of the growth hormone provide extended half-life to the modified growth hormone.

In some embodiments, a CTP sequence at the amino terminal end of a polypeptide, a CTP sequence at the carboxy terminal end of a growth hormone, and at least one additional CTP sequence attached in tandem to the CTP sequence at the carboxy terminus provide enhanced protection against degradation to a growth hormone. In some embodiments, a CTP sequence at the amino terminal end of a growth hormone, a CTP sequence at the carboxy terminal end of the growth hormone, and at least one additional CTP sequence attached in tandem to the CTP sequence at the carboxy terminus extend the half-life of the growth hormone. In some embodiments, a CTP sequence at the amino terminal end of a growth hormone, a CTP sequence at the carboxy terminal end of the growth hormone, and at least one additional CTP sequence attached in tandem to the CTP sequence at the carboxy terminus enhance the biological activity of the growth hormone.

In some embodiments, human growth hormone (hGH) is utilized according to the teachings of the present invention. In some embodiments, attachment of a CTP sequence to both the amino and carboxy termini of the hGH protein results in increased potency. In some embodiments, attachment of a CTP sequence to both the amino and carboxy termini of the hGH protein results in prolonged in vivo activity. A long-acting rhGH provided herein prolongs the half-live of protein drugs of molecular weight lower than 50,000 daltons, such as GH. In other embodiments, a long-acting rhGH provided herein enables interferons to exert their beneficial effects for a longer period of time.

In other embodiments, immunogenicity of a long-acting rhGH provided herein is equal to an isolated GH. In other embodiments, immunogenicity of a long-acting rhGH provided herein is comparable to an isolated GH. In other embodiments, modifying a GH as described herein with CTP peptides reduces immunogenicity of the GH. In other embodiments, a long-acting rhGH provided herein is as active as an isolated GH protein. In other embodiments, a long-acting rhGH provided herein is more active than an isolated GH. In other embodiments, a long-acting rhGH provided herein maximizes the growth hormone's protective ability against degradation while minimizing reductions in bioactivity.

In some embodiments, provided herein is a plurality of long-acting recombinant human growth hormone (long-acting rhGH) molecules, wherein each long-acting rhGH molecule comprises the amino acid sequence of mature human growth hormone (hGH) with one copy of CTP from the beta chain of human chorionic gonadotropin at the hGH N-terminus and two copies of CTP in tandem at the hGH C-terminus, and wherein the plurality comprises about 9 to O-glycans per intact long-acting rhGH molecule. In some embodiments, the long-acting rhGH comprises the amino acid sequence shown in SEQ ID NO: 2. In some embodiments, the long-acting rhGH is O-glycosylated on twelve to twenty serines. In some embodiments, plurality comprises a predominant glycoform having a molecular mass of about 40314 Da. In some embodiments, the plurality comprises additional predominant 0-glycoforms having molecular masses of about 39657 and 40970 Da. In some embodiments, the plurality comprises about 10-19 O-glycans per intact long-acting rhGH molecule. In some embodiments, a plurality comprises about 15 or 16 O-glycans per intact long-acting rhGH molecule. In some embodiments, the plurality comprises sialylated and di-sialylated core-1 O-glycans. In some embodiments, each CTP region comprises 0-5 hydroxy additions per intact somatrogon molecule.

### Methods of Treating

In some embodiments, the present teachings provide long-acting rhGH (e.g., somatrogon) for a once a week administration to a subject having a growth hormone deficiency. In some embodiments, a subject is a child. In other embodiments, a subject is a growth hormone deficient child. In some embodiments, the child is between 3 and 12 years of age. In other embodiments, the child is between 10 and 17 years of age. In some embodiments, the child is pre-pubertal and between 3 and 10 or 3 and 11 years of age depending upon whether the child is female or male respectively. In other embodiments, a subject is an adult. In other embodiments, a subject is a growth hormone deficient adult.

In some embodiments, the present teachings provide a method of treating a subject in need of GH therapy, comprising administering to said subject a therapeutically effective amount of a long-acting rhGH, thereby reducing the dosing frequency of a growth hormone in a subject. In other embodiments, a subject is a human subject. In some embodiments, a human subject is growth hormone deficient. In some embodiments, a subject is growth hormone deficient.

In some embodiments, a subject in need of GH therapy has been diagnosed with, and/or suffers from, a growth deficiency disorder such as, for example without limitation, isolated growth hormone deficiency (GHD), GH insufficiency or growth deficiency as part of multiple pituitary hormone deficiency, pediatric GHD, Prader-Willi Syndrome, Small for Gestational Age, Turner Syndrome, Idiopathic Short Stature, or Adult GHD.

In some embodiments, a subject is a growth hormone deficient child. In other embodiments, a subject is a pre-pubertal growth hormone deficient adult. In other embodiments, a subject is a pet. In other embodiments, a subject is a mammal. In other embodiments, a subject is a farm animal. In other embodiments, a subject is a dog. In other embodiments, a subject is a cat. In other embodiments, a subject is a monkey. In other embodiments, a subject is a horse. In other embodiments, a subject is a cow. In other embodiments, a subject is a mouse. In other embodiments, a subject is a rat. In some embodiments, a subject is male. In other embodiments, a subject is female.

In other embodiments, the present teachings provide a method of increasing insulin-like growth factor (IGF-1) levels in a subject, comprising administering to said subject a therapeutically effective amount of a long-acting rhGH, thereby increasing insulin-like growth factor (IGF-1) levels in a subject.

In other embodiments, the present teachings provide a method of maintaining insulin-like growth factor (IGF-1) levels in a subject, comprising administering to said subject a long-acting rhGH, thereby maintaining insulin-like growth factor (IGF-1) levels in a subject. In other embodiments, the IGF-1 levels are kept in a defined range, as further provided herein.

In other embodiments, the present teachings provide a method of increasing and maintaining insulin-like growth factor (IGF-1) levels within a defined range in a subject, comprising administering to said subject a long-acting rhGH, thereby increasing and maintaining insulin-like growth factor (IGF-1) levels within a defined range in a subject.

In some embodiments, provided herein is a method of achieving normal growth recovery of a pre-pubertal growth hormone deficient child, the method comprising administering a pharmaceutical composition comprising a CTP-modified growth hormone provided herein. In other embodiments, provided herein is a method of achieving growth recovery of a pre-pubertal growth hormone deficient child, the method comprising administering a pharmaceutical composition comprising a long-acting rhGH provided herein.

In some embodiments, the present teachings provide a method of treating a subject in need of GH therapy, the method comprising administering a long-acting growth hormone (e.g., comprising the amino acid sequence of SEQ ID NO:2) wherein the subject is small for gestational age, has Turner syndrome or has idiopathic short stature and optionally, wherein the long-acting growth hormone is administered at a dose of about 0.5 mg/kg body weight/week to about 1.5 mg/kg body weight/week, optionally at a dose of about 1.0 mg/kg body weight/week.

In some embodiments, the present teachings provide a method of inducing growth or weight gain in a subject, comprising administering to the subject a therapeutically effective amount of a long-acting rhGH comprising human growth hormone, one chorionic gonadotropin CTP attached to an amino terminus of said growth hormone, and two chorionic gonadotropin CTPs attached to a carboxy terminus of the growth hormone, thereby inducing growth or weight gain in a subject.

In some embodiments, a method comprises administering a long-acting rhGH to a subject previously administered a once daily rhGH therapy. In some embodiments, the subject had previously received a once daily rhGH therapy for one or more weeks. In some embodiments, a subject had previously received a once daily rhGH therapy for one or more months. In some embodiments, the subject had previously received a once daily rhGH therapy for one or more years. The once daily rhGH can be, for example without limitation, somatropin, including without limitation Genotropin®, Nutropin®, Humatrope®, Norditropin®, and Saizen®, a somatropin biosimilar such as for example Omnitrope®, Valtropin®, Zomacton®, and Eutropin®, somatrem, including Protropin, or a somatrem biosimilar. In some embodiments the daily rGH is Genotropin®, Nutropin®, Humatrope®, Norditropin®, or Saizen®. In some embodiments the daily rGH is Omnitrope®, Valtropin®, Zomacton®, Eutropin®, or other somatropin biosimilar.

In some embodiments, the methods comprise administering a long-acting rhGH to a subject who has not been previously administered a once daily rhGH therapy.

In some embodiments, a method of treating growth hormone deficiency in a first subject in need thereof comprises selecting a first subject with growth hormone deficiency, wherein the first subject has previously received a once daily recombinant human growth hormone (once daily rhGH) therapy; and administering an effective amount of a long-acting recombinant human growth hormone (long-acting rhGH) to the first subject, so that efficacy of the long-acting rhGH in the first subject is comparable to efficacy of the long-acting rhGH in a second subject who previously received only the long-acting rhGH and has not previously received the once daily rhGH therapy.

In some embodiments, a subject with a growth hormone deficiency is a female of three (3) to ≤10 years of age having impaired height and height velocity (HV) (e.g., annualized HV below the 25^{th} percentile for chronological age [HV≤−0.7 SDS]). In some embodiments, a subject with a growth hormone deficiency is a female of 3 to ≤10 years of age having an IGF-1 level≥1 SD below the age and sex-standardized mean IGF-1 level (e.g., SDS≤−1). In some embodiments, a subject with a growth hormone deficiency is a female of 3 to ≤10 years of age having impaired height and height velocity (HV) (e.g., annualized HV below the 25^{th} percentile for chronological age [HV≤−0.7 SDS]) and an IGF-1 level≥1 SD below the age and sex-standardized mean IGF-1 level (e.g., SDS≤−1). In some embodiments, the subject with a growth hormone deficiency has not received prior rhGH therapy.

In some embodiments, a subject with a growth hormone deficiency is a male of three (3) to ≤11 years of age having impaired height and height velocity (HV) (e.g., annualized HV below the 25^{th} percentile for chronological age [HV<−0.7 SDS]). In some embodiments, a subject with a growth hormone deficiency is a male of 3 to ≤11 years of age having an IGF-1 level≥1 SD below the age and sex-standardized mean IGF-1 level (e.g., SDS≤−1). In some embodiments, a subject with a growth hormone deficiency is a male of 3 to ≤11 years of age having impaired height and height velocity (HV) (e.g., annualized HV below the 25^{th} percentile for chronological age [HV<−0.7 SDS]) and an IGF-1 level≥1 SD below the age and sex-standardized mean IGF-1 level (e.g., SDS≤−1). In some embodiments, the subject with a growth hormone deficiency has not received prior rhGH therapy.

In some embodiments, a subject with a growth hormone deficiency is an adult having impaired height and height velocity (HV) (e.g., annualized HV below the 25^{th} percentile for chronological age [HV<−0.7 SDS]). In some embodiments, a subject with a growth hormone deficiency is an adult having an IGF-1 level≥1 SD below the age and sex-standardized mean IGF-1 level (e.g., SDS≤−1). In some embodiments, a subject with a growth hormone deficiency is an adult having impaired height and height velocity (HV) (e.g., annualized HV below the 25^{th} percentile for chronological age [HV<−0.7 SDS]) and an IGF-1 level≥1 SD below the age and sex-standardized mean IGF-1 level (e.g., SDS≤−1). In some embodiments, the subject with a growth hormone deficiency has not received prior rhGH therapy.

In some embodiments, a subject does not have active malignancy, a prior history of a malignancy or received radiation therapy or chemotherapy. In some embodiments, a subject does not have an acute illness such as for example, complications following open heart or abdominal surgery, multiple accidental trauma, or acute respiratory failure. In some embodiments, a subject does not have a body mass index (BMI) with an SDS of <−2 (age- and sex-standardized), anti-rhGH antibodies at screening, psychosocial dwarfism, a chromosomal abnormality (e.g., Turner syndrome, Laron syndrome, Noonan syndrome, Prader-Willi syndrome, Russell-Silver syndrome, SHOX mutations/deletions, or skeletal dysplasia), celiac disease, uncontrolled primary hypothyroidism, rickets or who was born small for their gestational age (birth weight/length SDS of <−2). In some embodiments, a subject does not have type 1 or type 2 diabetes mellitus and is not receiving standard of care, is noncompliant with their prescribed treatment, or is in poor metabolic control. In some embodiments, a subject is not receiving anabolic/sex steroid (except for drugs for ADHD or hormone replacement therapies), glucocorticoid therapy or inhaled budesonide at dose greater than 400 μg/day or equivalent. In some embodiments, a subject does not have >1 closed epiphyses, is not HIV-positive or with advanced diseases such as AIDS or tuberculosis, is not hypersensitive to components of study medication.

In some embodiments, a once daily rhGH is somatropin, somatrem, a somatropin biosimilar, or a somatrem biosimilar. In some embodiments, a subject is administered a once daily rhGH therapy at a dosage of about 0.16 mg to about 0.24 mg per kg body weight per week. In some embodiments, a subject received a once daily recombinant human growth hormone for at least three months. In some embodiments, a subject received a once daily recombinant human growth hormone for at least six months.

In some embodiments, a method or use further comprises monitoring glucose levels in the subject.

In some embodiments, a method or use demonstrates similar efficacy in a clinical study including participants divided into a test population and into a control population, wherein the test population receives (a) a once daily rhGH therapy for 12 months and then (b) a long-acting rhGH once weekly for 12 months, and the control population receives the long-acting rhGH once weekly for two years.

In some embodiments, an effective amount or dose of a long-acting rhGH is about 0.66 mg per kg body weight per week. In some embodiments, an effective amount or dose of a long-acting rhGH is about 0.56 mg per kg body weight per week. In some embodiments, an effective amount or dose of a long-acting rhGH is about 0.48 mg per kg body weight per week. In some embodiments, an effective amount or dose of a long-acting rhGH is about 0.36 mg per kg body weight per week. In some embodiments, an effective amount or dose of a long-acting rhGH is about 0.25 mg per kg body weight per week. In some embodiments, an effective amount or dose of a long-acting rhGH is about 0.16 mg per kg body weight per week. In some embodiments, an effective amount or dose of a long-acting rhGH is about 0.1 mg per kg body weight per week to about 1 mg per kg body weight per week.

In some embodiments, the long-acting rhGH is administered according to a dosage regimen comprising subcutaneous administration of about 0.66 mg per kg body weight once weekly at any time of day. In some embodiments, the long-acting rhGH is administered according to a dosage regimen comprising subcutaneous administration of about 0.56 mg per kg body weight once weekly at any time of day. In some embodiments, the long-acting rhGH is administered according to a dosage regimen comprising subcutaneous administration of about 0.48 mg per kg body weight once weekly at any time of day. In some embodiments, the long-acting rhGH is administered according to a dosage regimen comprising subcutaneous administration of about 0.36 mg per kg body weight once weekly at any time of day. In some embodiments, the long-acting rhGH is administered according to a dosage regimen comprising subcutaneous administration of about 0.25 mg per kg body weight once weekly at any time of day. In some embodiments, the long-acting rhGH is administered according to a dosage regimen comprising subcutaneous administration of about 0.16 mg per kg body weight once weekly at any time of day. In some embodiments, the long-acting rhGH is administered on the same day each week. In some embodiments, the time between two doses is at least three days. In some embodiments, the once daily rhGH therapy is administered at a dosage of about 0.16 to about 0.24 mg per kg body weight per week. In some embodiments, the long-acting rhGH is administered subcutaneously in the abdomen, thighs, buttocks, or upper arm.

In some embodiments, a long-acting rhGH is administered by subcutaneous injection. In some embodiments, a long-acting rhGH is administered once weekly at any time of day. In some embodiments, a long-acting rhGH is administered on the same day each week. In some embodiments, a long-acting rhGH comprises the amino acid sequence of mature human growth hormone (hGH) (e.g., SEQ ID NO:1) with one copy of CTP from the beta chain of human chorionic gonadotropin at the hGH N-terminus and two copies of CTP in tandem at the hGH C-terminus (e.g., somatrogon). In some embodiments, a long-acting rhGH comprises the amino acid sequence of SEQ ID NO: 2. In some embodiments, a CTP from the beta chain of human chorionic gonadotropin comprises the amino acid sequence of SEQ ID NO:3.

In some embodiments, efficacy of a long-acting rhGH in a first subject who previously received a once daily rhGH therapy is comparable to efficacy of the long-acting rhGH in a second subject, who previously received only the long-acting rhGH and did not previously receive the once daily rhGH therapy, when there is no significant difference in clinical measurements between the first subject and the second subject. In some embodiments, comparable efficacy includes a comparable safety profile. In some embodiments, a first subject may refer to a group of subjects similarly treated. In some embodiments, a second subject may refer to a group of subjects similarly treated.

In some embodiments, efficacy of a once daily rhGH or a long-acting rhGH is assessed by one or more clinical measurements: mean height velocity, annual height velocity, gain in height standard deviation score (SDS), body mass index, bone maturation, insulin growth factor-1 (IGF-1) SDS, insulin-like growth factor binding protein 3 IGFBP-3 SDS, pubertal status changed from Tanner 1, mean glucose, HbA1c, thyroid function, and cholesterol values. In some embodiments, annual height velocity, change in height standard deviation score (SDS), and bone maturation, which are assessed every 12 months. In some embodiments, biochemical endpoints including IGF-1 levels, IGF-1 SDS, IGFBP-3 levels, and IGFBP-3 SDS, are assessed on Day 4 after long-acting rhGH administration. In some embodiments, biochemical endpoints including IGF-1 levels, IGF-1 SDS, IGFBP-3 levels, and IGFBP-3 SDS, are assessed on day 4 following administration of a long-acting rhGH, including up to 24 hours before day 4 (i.e., from day 3 to day 4).

In some embodiments, efficacy is determined by monitoring glucose levels in a subject. In some embodiments, efficacy of a once daily rhGH or a long-acting rhGH is indicated by continued bone maturation.

In some embodiments, efficacy is determined by measuring or monitoring trunk fat mass, lean body mass, trunk fat mass as a percentage of total fat mass, IGF-1 levels, or a combination thereof, in a subject (e.g., an adult).

In some embodiments, a method of treating growth hormone deficiency in a population of participants in need thereof demonstrates similar efficacy in a clinical study including participants divided into a test population and into a control population, wherein the test population receives (a) the once daily rhGH therapy for 12 months and then (b) the long-acting rhGH once weekly for 12 months, and the control population receives the long-acting rhGH once weekly for two years.

In some embodiments, a method of treating growth hormone deficiency in a subject in need thereof, comprises i) administering a long-acting recombinant human growth hormone (rhGH) to the subject at an initial dose level; ii) taking a measurement of an IGF-1 level in the subject; and iii) administering the long-acting rhGH to the subject at a modified dose level based on the IGF-1 level in the subject. In some embodiments, a long-acting rhGH comprises the amino acid sequence of SEQ ID NO:2.

In some embodiments, a long-acting rhGH is administered once a week to a female of three (3) to ≤10 years of age at an initial dose level or at a modified dose level of about 0.66 mg/kg, about 0.56 mg/kg, about 0.48 mg/kg, about 0.36 mg/kg, about 0.25 mg/kg, or about 0.16 mg/kg. In some embodiments, a subject with a growth hormone deficiency has impaired height and height velocity (HV) (e.g., annualized HV below the 25^{th} percentile for chronological age [HV has a SDS of <−0.7]) and an IGF-1 level SDS≥1 SD below the age and sex-standardized mean IGF-1 level (e.g., SDS≤−1). In some embodiments, a subject with growth hormone deficiency has not received prior rhGH therapy.

In some embodiments, a long-acting rhGH is administered once a week to a male of three (3) to ≤11 years of age at an initial dose level or at a modified dose level of about 0.66 mg/kg, about 0.56 mg/kg, about 0.48 mg/kg, about 0.36 mg/kg, about 0.25 mg/kg, or about 0.16 mg/kg. In some embodiments, a subject with a growth hormone deficiency has impaired height and height velocity (HV) (e.g., annualized HV below the 25^{th} percentile for chronological age [HV has a SDS of <−0.7]) and an IGF-1 level SDS≥1 SD below the age and sex-standardized mean IGF-1 level (e.g., SDS≤−1). In some embodiments, a subject with growth hormone deficiency has not received prior rhGH therapy.

In some embodiments, a long-acting rhGH is administered once a week to an adult at an initial dose level or at a modified dose level of about 1 mg/week, about 1.2 mg/week, about 1.45 mg/week, about 1.82 mg/week, about 2 mg/week, about 2.18 mg/week, about 2.5 mg/week, about 2.54 mg/week, about 2.75 mg/week, about 2.9 mg/week, about 3 mg/week, about 3.25 mg/week, about 3.5 mg/week, about 4 mg/week, about 4.5 mg/week, about 5 mg/week, or about 5.5 mg/week. In some embodiments, a subject with a growth hormone deficiency has impaired height and height velocity (HV) (e.g., annualized HV below the 25^{th} percentile for chronological age [HV has a SDS of <−0.7]) and an IGF-1 level SDS≥1 SD below the age and sex-standardized mean IGF-1 level (e.g., SDS≤−1). In some embodiments, a subject with growth hormone deficiency has not received prior rhGH therapy.

In some embodiments, an initial dose of a long-acting rhGH is about 0.66 mg/kg body weight/week. In some embodiments, an initial dose of a long-acting rhGH for a pediatric patient or subject is about 0.66 mg/kg body weight/week. In some embodiments, a method of treating growth hormone deficiency in a subject with a long acting rhGH includes decreasing an initial dose of long-acting rhGH based on two repeated day 4 (−1) levels (i.e., from day 3 to day 4) of IGF-1>+2.0 SDS. In some embodiments, day 4 (−1) is understood to refer to days after administration of a long-acting rhGH. In some embodiments, day 4 (−1) is understood to refer to optimally about 96 hours after administration of a long-acting rhGH.

In some embodiments, an IGF-1 level is measured in serum or plasma. In some embodiments, an IGF-1 level is measured on day 4 following administration of an initial dose of a long-acting rhGH, including up to 24 hours before day 4 (i.e., from day 3 to day 4). In some embodiments, an IGF-1 level in a subject has an SDS of >+2 after administration of a long-acting rhGH at an initial dose level. In some embodiments, an IGF-1 level is measured from day 3 to day 4 after administration of a long-acting rhGH at an initial dose level and the IGF-1 level in a subject has an SDS of >+2.

In some embodiments, a method of treating growth hormone deficiency in a subject with a long-acting rhGH includes decreasing an initial dose of long-acting rhGH based on two repeated day 4 (−1) levels (i.e., from day 3 to day 4) of IGF-1>+2.0 SDS. Day 4 (−1) is understood to refer to days after administration of a long-acting rhGH. In some embodiments, day 4 (−1) is understood to refer to optimally about 96 hours after administration of a long-acting rhGH.

In some embodiments, an IGF-1 level in a subject who is receiving weekly administration of a long-acting rhGH at an initial dose level has an SDS>+2 on two consecutive measurements taken 4 to 6 weeks apart. Two consecutive measurements of IGF-1 taken 4 to 6 weeks apart is understood to include taking a second measurement of IGF-1 within 4 to 6 weeks after the first measurement of IGF-1.

In some embodiments, when an IGF-1 level in a subject who is receiving weekly administration of a long-acting rhGH at an initial dose level has an SDS>+2 on two consecutive measurements taken 4 to 6 weeks apart, the subject is subsequently administered the long-acting rhGH at a modified dose level. In some embodiments, a modified dose level is 15% lower than an initial dose level. In some embodiments, a modified dose level is about 0.56 mg per kg body weight per week.

In some embodiments, a method further comprises taking a measurement of an IGF-1 level in a subject at least 4 weeks after administration of a long acting rhGH at a modified dose level. In some embodiments, an IGF-1 level in a subject has an SDS>+2 after administration of a long-acting rhGH at a modified dose level.

In some embodiments, a method further comprises administering a long-acting rhGH to a subject at a further modified dose level when an IGF-1 level in the subject has a SDS>+2 after administering the long-acting rhGH at a modified dose level (e.g., 15% lower than an initial dose level). In some embodiments, a further modified dose level of long-acting rhGH is 30% lower than an initial dose level of the long-acting rhGH. In some embodiments, a further modified dose level of long-acting rhGH is 15% lower than a modified dose level of long-acting rhGH. In some embodiments, a further modified dose level of a long-acting rhGH is administered once per week. In some embodiments, a further modified dose level of long-acting rhGH is about 0.48 mg per kg body weight per week.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.66 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; and iii) administering the long-acting rhGH to the subject at a modified dose level wherein the modified dose level is about 15% lower or is between 10% to 20% lower than the initial dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.56 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; and iii) administering the long-acting rhGH to the subject at a modified dose level wherein the modified dose level is about 15% lower, or is between 10% to 20% lower than the initial dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.48 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; and iii) administering the long-acting rhGH to the subject at a modified dose level wherein the modified dose level is about 15% lower, or is between 10% to 20% lower than the initial dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.36 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; and iii) administering the long-acting rhGH to the subject at a modified dose level wherein the modified dose level is about 15% lower, or is between 10% to 20% lower than the initial dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.25 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; and iii) administering the long-acting rhGH to the subject at a modified dose level wherein the modified dose level is about 15% lower, or is between 10% to 20% lower, than the initial dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.16 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; and iii) administering the long-acting rhGH to the subject at a modified dose level wherein the modified dose level is about 15% lower, or is between 10% to 20% lower, than the initial dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.66 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; iii) administering the long-acting rhGH to the subject at a modified dose level wherein the modified dose level is about 15% lower, or is between 10% to 20% lower, than the initial dose level; iv) taking at least one measurement of an IGF-1 level in the subject at least 4 weeks after administering the modified dose level, wherein the at least one measurement of an IGF-1 level in the subject has an SDS of >+2; and v) administering the long-acting rhGH to the subject at a further modified dose level wherein the further modified dose level is about 15% lower, or is between 10% to 20% lower, than the modified dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.56 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; iii) administering the long-acting rhGH to the subject at a modified dose level wherein the modified dose level is about 15% lower, or is between 10% to 20% lower, than the initial dose level; iv) taking at least one measurement of an IGF-1 level in the subject at least 4 weeks after administering the modified dose level, wherein the at least one measurement of an IGF-1 level in the subject has an SDS of >+2; and v) administering the long-acting rhGH to the subject at a further modified dose level wherein the further modified dose level is about 15% lower, or is between 10% to 20% lower, than the modified dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.48 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; iii) administering the long-acting rhGH to the subject at a modified dose level wherein the modified dose level is about 15% lower, or is between 10% to 20% lower, than the initial dose level; iv) taking at least one measurement of an IGF-1 level in the subject at least 4 weeks after administering the modified dose level, wherein the at least one measurement of an IGF-1 level in the subject has an SDS of >+2; and v) administering the long-acting rhGH to the subject at a further modified dose level wherein the further modified dose level is about 15% lower, or is between 10% to 20% lower, than the modified dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.36 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; iii) administering the long-acting rhGH to the subject at a modified dose level wherein the modified dose level is about 15% lower, or is between 10% to 20% lower, than the initial dose level; iv) taking at least one measurement of an IGF-1 level in the subject at least 4 weeks after administering the modified dose level, wherein the at least one measurement of an IGF-1 level in the subject has an SDS of >+2; and v) administering the long-acting rhGH to the subject at a further modified dose level wherein the further modified dose level is about 15% lower, or is between 10% to 20% lower, than the modified dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.25 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; iii) administering the long-acting rhGH to the subject at a modified dose level wherein the modified dose level is about 15% lower, or is between 10% to 20% lower, than the initial dose level; iv) taking at least one measurement of an IGF-1 level in the subject at least 4 weeks after administering the modified dose level, wherein the at least one measurement of an IGF-1 level in the subject has an SDS of >+2; and v) administering the long-acting rhGH to the subject at a further modified dose level wherein the further modified dose level is about 15% lower, or is between 10% to 20% lower, than the modified dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose of about 0.16 mg per kg of body weight per week; ii) taking at least two measurements of an insulin growth factor-1 (IGF-1) level in the subject, wherein the IGF-1 level in the subject on two consecutive measurements taken 4 to 6 weeks apart each has a standard deviation score (SDS) of >+2; iii) administering the long-acting rhGH to the subject at a modified dose level wherein the modified dose level is about 15% lower, or is between 10% to 20% lower, than the initial dose level; iv) taking at least one measurement of an IGF-1 level in the subject at least 4 weeks after administering the modified dose level, wherein the at least one measurement of an IGF-1 level in the subject has an SDS of >+2; and v) administering the long-acting rhGH to the subject at a further modified dose level wherein the further modified dose level is about 15% lower, or is between 10% to 20% lower, than the modified dose level. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose level of about 0.66 mg per kg body weight per week; ii) taking a measurement of an IGF-1 level in the subject at day 3 to day 4 after administering the long-acting rhGH, wherein the IGF-1 level in the subject on two consecutive measurements taken 4 to 6 weeks apart is >+2 SDS; iii) administering the long-acting rhGH to the subject at a modified dose level, wherein the modified dose level is about 15% lower, or is between 10% to 20% lower, than the initial dose level; iv) taking a measurement of the IGF-1 level in the subject at least 4 weeks after administering the modified dose level, wherein the IGF-1 level at least 4 weeks after administering the modified dose level is >+2 SDS; v) administering the long-acting rhGH to the subject at a further modified dose level, wherein the further modified dose level is about 15% lower, or is between 10% to 20% lower, than the modified dose level. In some embodiments, a long-acting rhGH is administered to a subject at a further modified dose level, wherein the further modified dose level is 30% lower than the initial dose level of long-acting rhGH. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose level of about 0.56 mg per kg body weight per week; ii) taking a measurement of an IGF-1 level in the subject at day 3 to day 4 after administering the long-acting rhGH, wherein the IGF-1 level in the subject on two consecutive measurements taken 4 to 6 weeks apart is >+2 SDS; iii) administering the long-acting rhGH to the subject at a modified dose level, wherein the modified dose level is about 15% lower, or is between 10% to 20% lower, than the initial dose level; iv) taking a measurement of the IGF-1 level in the subject at least 4 weeks after administering the to modified dose level, wherein the IGF-1 level at least 4 weeks after administering the modified dose level is >+2 SDS; v) administering the long-acting rhGH to the subject at a further modified dose level, wherein the further modified dose level is about 15% lower, or is between 10% to 20% lower, than the modified dose level. In some embodiments, a long-acting rhGH is administered to a subject at a further modified dose level, wherein the further modified dose level is 30% lower than the initial dose level of long-acting rhGH. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose level of about 0.48 mg per kg body weight per week; ii) taking a measurement of an IGF-1 level in the subject at day 3 to day 4 after administering the long-acting rhGH, wherein the IGF-1 level in the subject on two consecutive measurements taken 4 to 6 weeks apart is >+2 SDS; iii) administering the long-acting rhGH to the subject at a modified dose level, wherein the modified dose level is about 15% lower, or is between 10% to 20% lower, than the initial dose level; iv) taking a measurement of the IGF-1 level in the subject at least 4 weeks after administering the modified dose level, wherein the IGF-1 level at least 4 weeks after administering the modified dose level is >+2 SDS; v) administering the long-acting rhGH to the subject at a further modified dose level, wherein the further modified dose level is about 15% lower, or is between 10% to 20% lower, than the modified dose level. In some embodiments, a long-acting rhGH is administered to a subject at a further modified dose level, wherein the further modified dose level is 30% lower than the initial dose level of long-acting rhGH. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose level of about 0.36 mg per kg body weight per week; ii) taking a measurement of an IGF-1 level in the subject at day 3 to day 4 after administering the long-acting rhGH, wherein the IGF-1 level in the subject on two consecutive measurements taken 4 to 6 weeks apart is >+2 SDS; iii) administering the long-acting rhGH to the subject at a modified dose level, wherein the modified dose level is about 15% lower, or is between 10% to 20% lower, than the initial dose level; iv) taking a measurement of the IGF-1 level in the subject at least 4 weeks after administering the modified dose level, wherein the IGF-1 level at least 4 weeks after administering the modified to dose level is >+2 SDS; v) administering the long-acting rhGH to the subject at a further modified dose level, wherein the further modified dose level is about 15% lower, or is between 10% to 20% lower, than the modified dose level. In some embodiments, a long-acting rhGH is administered to a subject at a further modified dose level, wherein the further modified dose level is 30% lower than the initial dose level of long-acting rhGH. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose level of about 0.25 mg per kg body weight per week; ii) taking a measurement of an IGF-1 level in the subject at day 3 to day 4 after administering the long-acting rhGH, wherein the IGF-1 level in the subject on two consecutive measurements taken 4 to 6 weeks apart is >+2 SDS; iii) administering the long-acting rhGH to the subject at a modified dose level, wherein the modified dose level is about 15% lower, or is between 10% to 20% lower, than the initial dose level; iv) taking a measurement of the IGF-1 level in the subject at least 4 weeks after administering the modified dose level, wherein the IGF-1 level at least 4 weeks after administering the modified dose level is >+2 SDS; v) administering the long-acting rhGH to the subject at a further modified dose level, wherein the further modified dose level is about 15% lower, or is between 10% to 20% lower, than the modified dose level. In some embodiments, a long-acting rhGH is administered to a subject at a further modified dose level, wherein the further modified dose level is 30% lower than the initial dose level of long-acting rhGH. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in a subject in need thereof comprises i) administering a long-acting recombinant human growth hormone (rhGH) comprising the amino acid sequence of SEQ ID NO:2 to the subject at an initial dose level of about 0.16 mg per kg body weight per week; ii) taking a measurement of an IGF-1 level in the subject at day 3 to day 4 after administering the long-acting rhGH, wherein the IGF-1 level in the subject on two consecutive measurements taken 4 to 6 weeks apart is >+2 SDS; iii) administering the long-acting rhGH to the subject at a modified dose level, wherein the modified dose level is about 15% lower, or is between 10% to 20% lower, than the initial dose level; iv) taking a measurement of the IGF-1 level in the subject at least 4 weeks after administering the modified dose level, wherein the IGF-1 level at least 4 weeks after administering the modified dose level is >+2 SDS; v) administering the long-acting rhGH to the subject at a further modified dose level, wherein the further modified dose level is about 15% lower, or is between 10% to 20% lower, than the modified dose level. In some embodiments, a long-acting rhGH is administered to a subject at a further modified dose level, wherein the further modified dose level is 30% lower than the initial dose level of long-acting rhGH. In some embodiments, the subject is a pediatric subject. In some further embodiments, the pediatric subject is a female of 3 to ≤10 years of age or a male of 3 to ≤11 years of age.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age; ii) taking at least one measurement of an IGF-1 level in the subject wherein the IGF-1 level in the subject has a standard deviation score (SDS) of >+1.5; and iii) administering the long-acting rhGH to the subject at a modified dose level, wherein the modified dose level is about 0.5 mg/week lower than the initial dose level when the IGF-1 level is the subject has an SDS value of >+1.5. In some embodiments, the method further comprises taking an additional at least one measurement of an IGF-1 level in a subject after administering the long-acting rhGH at the modified dose level, and administering the long-acting rhGH to the subject at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age; ii) taking at least one measurement of an IGF-1 level in the subject wherein the IGF-1 level in the subject has a standard deviation score (SDS) of >+1.5; and iii) administering the long-acting rhGH to the subject at a modified dose level, wherein the modified dose level is about 0.75 mg/week lower than the initial dose level when the IGF-1 level is the subject has an SDS value of >+1.5. In some embodiments, the method further comprises taking an additional at least one measurement of an IGF-1 level in a subject after administering the long-acting rhGH at the modified dose level, and administering the long-acting rhGH to the subject at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age; ii) taking at least one measurement of an IGF-1 level in the subject wherein the IGF-1 level in the subject has a standard deviation score (SDS) of <−0.5; and iii) administering the long-acting rhGH to the subject at a modified dose level, wherein the modified dose level is about 1.0 mg/week higher than the initial dose level when the IGF-1 level is the subject has an SDS value of <−0.5. In some embodiments, the method further comprises taking an additional at least one measurement of an IGF-1 level in a subject after administering the long-acting rhGH at the modified dose level, and administering the long-acting rhGH to the subject at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age; ii) taking at least one measurement of an IGF-1 level in the subject wherein the IGF-1 level in the subject has a standard deviation score (SDS) of <−0.5; and iii) administering the long-acting rhGH to the subject at a modified dose level, wherein the modified dose level is about 1.5 mg/week higher than the initial dose level when the IGF-1 level is the subject has an SDS value of <−0.5. In some embodiments, the method further comprises taking an additional at least one measurement of an IGF-1 level in a subject after administering the long-acting rhGH at the modified dose level, and administering the long-acting rhGH to the subject at a further modified dose level.

In some embodiments, a long-acting rhGH is administered once a week at an initial dose level, at a modified dose level or at a further modified dose level. In some embodiments, a subject is an adult or a child. In some embodiments an IGF-1 level is measured in serum or plasma. In some embodiments, a modified dose level is about 0.56 mg per kg body weight per week. In some embodiments, a further modified dose level is about 0.48 mg per kg body weight per week.

In other embodiments, a long-acting rhGH is administered to a subject in a dose ranging from about 2 mg to about 24 mg. In other embodiments, a long-acting rhGH is administered to a subject in a dose ranging from about 1 mg to about 11 mg. In other embodiments, a long-acting rhGH is administered to a subject in a dose ranging from about 2 mg to about 12 mg. In other embodiments, a long-acting rhGH is administered to a subject in a dose ranging from about 0.5 mg to about 60 mg. In other embodiments, a long-acting rhGH is administered to a subject in a dose ranging from about 0.5 mg to about 30 mg. In other embodiments, a long-acting rhGH is administered to subject in a dose ranging from about 0.5 mg to about 110 mg.

In some embodiments, a long-acting rhGH is administered to a subject in a dose of about 0.13 mg, about 0.25 mg, about 0.36 mg, about 0.48 mg, about 0.56 or about 0.66 mg per kg body weight per week. In some embodiments, a long-acting rhGH is administered to a subject in a dose of about 0.66 mg per kg body weight. In some embodiments, a long-acting rhGH is administered to a subject in a dosage of about 0.66 mg per kg body weight per week.

In some embodiments, a dosage regimen comprises administering a long-acting rhGH (e.g., somatrogon) in a dose of about 0.66 mg per kg body weight per week.

In some embodiments, a recommended dose of a long-acting rhGH (e.g., somatrogon) is about 0.66 mg/kg body weight administered once weekly by subcutaneous (SC) injection. In some embodiments, for patients switching from daily medicinal growth hormone products, weekly therapy with a long-acting rhGH (e.g., somatrogon) may be initiated at a dose of about 0.66 mg/kg/wk on the day following the patient's last daily injection. In some embodiments, regular monitoring of IGF-1 concentrations is recommended during treatment with a long-acting rhGH (e.g., somatrogon). In some embodiments, dosage of a long-acting rhGH (e.g., somatrogon) may be adjusted as necessary, based on growth velocity, body weight, and serum insulin-like growth factor 1 (IGF-1) concentrations. In some embodiments, when monitoring for IGF-1 concentrations, samples should be drawn 4 days after the prior dose of a long-acting rhGH (e.g., somatrogon), optionally target IGF-1 standard deviation score (SDS) should be the upper normal range not exceeding 2 SDS. In some embodiments, when a patient's blood IGF-1 concentration exceeds the mean reference value for their age and sex by more than 2 SDS, a dose of a long-acting rhGH (e.g., somatrogon) should be reduced by 15%. In some embodiments, a patient may require more than one dose reduction. In some embodiments, growth rates during the first year of treatment with a long-acting rhGH (e.g., somatrogon) should be monitored.

In some embodiments, a long-acting rhGH is administered to an adult male subject 50 years old or younger at a dosage of about 2.5 mg/week or 1.82 mg/wk. In some embodiments, a long-acting rhGH is administered to an adult male subject older than 50 years old at a dosage of about 2 mg/week or 1.45 mg/week. In some embodiments, a long-acting rhGH is administered to an adult female subject 50 years old or younger at a dosage of about 3 mg/week or 2.18 mg/wk. In some embodiments, a long-acting rhGH is administered to an adult female subject older than 50 years old at a dosage of about 2.5 mg/week or 1.2 mg/week. In some embodiments, an adult female subject is not on oral estrogen. In some embodiments, an adult female subject is on oral estrogen. In other embodiments, a long-acting rhGH is administered to an adult female subject 50 years old or younger at a dosage of about 4 mg/week or 2.9 mg/week. In other embodiments, a long-acting rhGH is administered to an adult female subject 50 years old or younger who is on oral estrogen at a dosage of about 4 mg/week or about 2.9 mg/week. In other embodiments, a long-acting rhGH is administered to an adult female subject older than 50 years old at a dosage of about 3.5 mg/week or 2.54 mg/week. In other embodiments, a long-acting rhGH is administered to an adult female subject older than 50 years old who is on oral estrogen at a dosage of about 3.5 mg/week or about 2.54 mg/week

In some embodiments, a dosage regimen comprises administering a long-acting rhGH (e.g., somatrogon) to an adult at an initial dose level, wherein the initial dose level ranges from about 1 mg/week to about 5 mg/week (e.g., about 1 mg/week, about 1.2 mg/week, about 1.45 mg/week, about 1.82 mg/week, about 2 mg/week, about 2.18 mg/week, about 2.5 mg/week, about 2.54 mg/week, about 2.9 mg/week, about 3 mg/week, about 3.25 mg/week, about 3.5 mg/week, about 4 mg/week, about 4.5 mg/week, or about 5 mg/week).

In some embodiments, a method of treating growth hormone deficiency comprises administering a long-acting recombinant human growth hormone (e.g., comprising the amino acid sequence of SEQ ID NO:2) to a subject (e.g., an adult) at an initial dose level, monitoring the subject for an adverse event (AE), and administering the long-acting rhGH to the subject at a modified dose level that is 25% lower than the initial dose level if the adverse event is moderate. In some embodiments, the initial dose is 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

In some embodiments, a method of treating growth hormone deficiency comprises administering a long-acting recombinant human growth hormone (e.g., comprising the amino acid sequence of SEQ ID NO:2) to a subject (e.g., an adult) at an initial dose level, monitoring the subject for an adverse event (AE), and administering the long-acting rhGH to the subject at a modified dose level that is 25% lower than the initial dose level if the adverse event is moderate. In some embodiments, an adverse event is at least one of the following: edema, hypertension, carpal tunnel and glucose intolerance.

In some embodiments, a method of treating growth hormone deficiency comprises administering a long-acting recombinant human growth hormone (e.g., comprising the amino acid sequence of SEQ ID NO:2) to a subject (e.g., an adult) at an initial dose level, monitoring the subject for an adverse event (AE), and administering the long-acting rhGH to the subject at a modified dose level that is 50% lower than the initial dose level if the adverse event is severe. In some embodiments, a modified dose level is a skipped dose if the adverse event is severe. In some embodiments, the initial dose is 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

In some embodiments, a method of treating growth hormone deficiency comprises administering a long-acting recombinant human growth hormone (e.g., comprising the amino acid sequence of SEQ ID NO:2) to a subject (e.g., an adult) at an initial dose level, monitoring the subject for an adverse event (AE), and administering the long-acting rhGH to the subject at a modified dose level that is 50% lower than the initial dose level if the adverse event is severe. In some embodiments, a modified dose level is a skipped dose if the adverse event is severe. In some embodiments, an adverse event is at least one of the following: edema, hypertension, carpal tunnel and glucose intolerance.

In some embodiments, an AE is characterized as mild when the AE results in transient or mild discomfort; there is no limitation in activity; and no medical intervention and/or therapy is required. In some embodiments, an AE is characterized as moderate when the AE results in a mild to moderate limitation in activity, some assistance may be needed; and no or minimal medical intervention and/or therapy is required. In some embodiments, an AE is characterized as severe when the AE results in a marked limitation in activity, some assistance is usually required; medical intervention and/or therapy is required; and hospitalization is possible.

In some embodiments, if an adverse event is moderate, a dose level (e.g., an initial dose level, a modified dose level, a further modified dose level, etc) may be reduced by 25% such that the new dose ranges from about 1.5 mg/week to about 3 mg/week based on age, gender and/or estrogen status. In some embodiments, the dose level (e.g., an initial dose level, a modified dose level, a further modified dose level, etc) that is reduced by 25% is about 2 mg/week, about 2.18 mg/week, about 2.5 mg/week, about 2.54 mg/week, about 2.9 mg/week, about 3 mg/week, about 3.25 mg/week, or about 3.5 mg/week.

In some embodiments, if an adverse event is severe, a dose level (e.g., an initial dose level, a modified dose level, a further modified dose level, etc) may be reduce by 50% such that the new dose ranges from about 1 mg/week to about 2 mg/week based on age, gender and/or estrogen status. In some embodiments, the dose level (e.g., an initial dose level, a modified dose level, a further modified dose level, etc) that is reduced by 50% is about 2 mg/week, about 2.18 mg/week, about 2.5 mg/week, about 2.54 mg/week, about 2.9 mg/week, about 3 mg/week, about 3.25 mg/week, about 3.5 mg/week, or about 4 mg/week.

In some embodiments, a method of treating growth hormone deficiency in an adult subject in need thereof, comprises i) administering a long-acting recombinant human growth hormone (rhGH) to the subject at an initial dose level; ii) taking a measurement an IGF-1 level in the subject; and iii) administering the long-acting rhGH to the subject at a modified dose level based on the IGF-1 level in the subject. In some embodiments, a long-acting rhGH comprises the amino acid sequence of SEQ ID NO:2.

In some embodiments, a long-acting rhGH is administered once a week at an initial dose level or at a modified dose level. In some embodiments, a subject with growth hormone deficiency has not received prior rhGH therapy. In some embodiments, an initial dose of long-acting rhGH ranges from about 1 mg/week to about 5 mg/week for an adult with growth hormone deficiency. In some embodiments, an initial dose of long-acting rhGH is: about 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

In some embodiments, an IGF-1 level is measured in serum or plasma. In some embodiments, an IGF-1 level is measured at day 3 to day 4 after administration of a long-acting rhGH at an initial dose level. In some embodiments, an IGF-1 level in a subject has a SDS of >+1.5 after administration of a long-acting rhGH at an initial dose level. In some embodiments, an IGF-1 level in a subject measured at day 3 to day 4 after administration of a long-acting rhGH at an initial dose level has an SDS of >+1.5.

In some embodiments, when an IGF-1 level in a subject who is receiving weekly administration of a long-acting rhGH at an initial dose level has an SDS of >+1.5, the subject is subsequently administered the long-acting rhGH at a modified dose level. In some embodiments, a modified dose level is about 0.5 mg/week lower than an initial dose level. In some embodiments, a modified dose level is about 0.75 mg/week lower than an initial dose level.

In some embodiment, when an IGF-1 level in an adult male 50 years of age or less who is receiving weekly administration of a long-acting rhGH at an initial dose level of about 2.5 mg per week has an SDS of >+1.5, the adult male is subsequently administered the long-acting rhGH at a modified dose level of about 2.0 mg per week.

In some embodiments, when an IGF-1 level in an adult male greater than 50 years of age who is receiving weekly administration of a long-acting rhGH at an initial dose level of about 2.0 mg per week has an SDS of >+1.5, the adult male is subsequently administered the long-acting rhGH at a modified dose level of about 1.5 mg per week.

In some embodiments, when an IGF-1 level in an adult female 50 years of age or less who is not on oral estrogen but who is receiving weekly administration of a long-acting rhGH at an initial dose level of about 3.0 mg per week has an SDS of >+1.5, the adult female is subsequently administered the long-acting rhGH at a modified dose level of about 2.5 mg per week.

In some embodiments, when an IGF-1 level in an adult female greater than 50 years of age who is not on oral estrogen and who is receiving weekly administration of a long-acting rhGH at an initial dose level of about 2.5 mg per week has an SDS of >+1.5, the adult female is subsequently administered the long-acting rhGH at a modified dose level of about 2.0 mg per week.

In some embodiments, when an IGF-1 level in an adult female 50 years of age or less who is on oral estrogen and who is receiving weekly administration of a long-acting rhGH at an initial dose level of about 4.0 mg per week has an SDS of >+1.5, the adult female is subsequently administered the long-acting rhGH at a modified dose level of about 3.25 mg per week.

In some embodiments, when an IGF-1 level in an adult female greater than 50 years of age who is on oral estrogen and who is receiving weekly administration of a long-acting rhGH at an initial dose level of about 3.5 mg per week has an SDS of >+1.5, the adult female is subsequently administered the long-acting rhGH at a modified dose level of about 2.75 mg per week.

In some embodiments, a method further comprises administering a long-acting rhGH to a subject (e.g., adult male, adult female on oral estrogen, adult female not on oral estrogen) at a further modified dose level when an IGF-1 level in the subject has an SDS of >+1.5 when measured 3 to 4 days after administration of the long-acting rhGH at a modified dose level. In some embodiments, a further modified dose level is about 0.5 mg/week lower than a modified dose if the subject is a male or if the subject is a female who is not being treated with estrogen. In some embodiments, a further modified dose level is about 1.0 mg/week lower than an initial dose level if the subject is a male or if the subject is a female who it not being treated with estrogen. In some embodiments, a further modified dose level is about 0.75 mg/week lower than a modified dose if the subject is a female who is being treated with estrogen. In some embodiments, a further modified dose level is about 1.5 mg/week lower than an initial dose level if the subject is a female who is being treated with estrogen.

In some embodiments, an IGF-1 level in a subject has an SDS of <−0.5 after administration of a long-acting rhGH at an initial dose level. In some embodiments, an IGF-1 level in an adult subject measured at day 3 to day 4 after administration of a long-acting rhGH at an initial dose level has an SDS of <−0.5.

In some embodiments, when an IGF-1 level in a subject who is receiving weekly administration of a long-acting rhGH at an initial dose level has a SDS of <−0.5, the subject is subsequently administered the long-acting rhGH at a modified dose level. In some embodiments, a modified dose level is about 1.0 mg/week higher than an initial dose level. In some embodiments, a modified dose level is about 1.5 mg/week higher than an initial dose level.

In some embodiments, when an IGF-1 level in an adult male 50 years of age or less who is receiving weekly administration of a long-acting rhGH at an initial dose level of about 2.5 mg per week has a SDS of <−0.5, the adult male is subsequently administered the long-acting rhGH at a modified dose level of about 3.5 mg per week.

In some embodiments, when an IGF-1 level in an adult male greater than 50 years of age who is receiving weekly administration of a long-acting rhGH at an initial dose level of about 2.0 mg per week has an SDS of <−0.5, the adult male is subsequently administered the long-acting rhGH at a modified dose level of about 3.0 mg per week.

In some embodiments, when an IGF-1 level in an adult female 50 years of age or less who is not on oral estrogen but who is receiving weekly administration of a long-acting rhGH at an initial dose level of about 3.0 mg per week has an SDS of <−0.5, the adult female is subsequently administered the long-acting rhGH at a modified dose level of about 4.0 mg per week.

In some embodiments, when an IGF-1 level in an adult female greater than 50 years of age who is not on oral estrogen and who is receiving weekly administration of a long-acting rhGH at an initial dose level of about 2.5 mg per week has an SDS of <−0.5, the adult female is subsequently administered the long-acting rhGH at a modified dose level of about 3.5 mg per week.

In some embodiments, when an IGF-1 level in an adult female 50 years of age or less who is on oral estrogen and who is receiving weekly administration of a long-acting rhGH at an initial dose level of about 4.0 mg per week has an SDS of <−0.5, the adult female is subsequently administered the long-acting rhGH at a modified dose level of about 5.5 mg per week.

In some embodiments, when an IGF-1 level in an adult female greater than 50 years of age who is on oral estrogen and who is receiving weekly administration of a long-acting rhGH at an initial dose level of about 3.5 mg per week has an SDS of <−0.5, the adult female is subsequently administered the long-acting rhGH at a modified dose level of about 5.0 mg per week.

In some embodiments, a method further comprises administering a long-acting rhGH to a subject (e.g., adult male, adult female on oral estrogen, adult female not on oral estrogen) at a further modified dose level when an IGF-1 level in the subject has an SDS of <−0.5 when measured 3 to 4 days after administering the long-acting rhGH at a modified dose level. In some embodiments, a further modified dose level is about 1.0 mg/week higher than a modified dose if the subject is a male or if the subject is a female who is not being treated with estrogen. In some embodiments, a further modified dose level is about 2.0 mg/week higher than an initial dose level if the subject is a male or if the subject is a female who it not being treated with estrogen. In some embodiments, a further modified dose level is about 1.5 mg/week higher than a modified dose if the subject is a female who is being treated with estrogen. In some embodiments, a further modified dose level is about 3.0 mg/week higher than an initial dose level if the subject is a female who is being treated with estrogen.

In some embodiments, a method may further comprise administering the long-acting rhGH one, two, three, four, five, six, seven, eight, nine, ten or more times, taking a measurement of an IGF-1 level in a subject at day 3 to day 4 after each administration and reducing the dose level of long-acting rhGH by about 0.5 mg/week or about 0.75 mg/week if the IGF-1 level has an SDS of >+1.5 or increasing the dose level of long-acting rhGH by about 1.0 mg/week or about 1.5 mg/week if the IGF-1 level has an SDS of <−0.5.

In some embodiments, a subject (e.g., an adult) treated with a long acting recombinant growth hormone has a decreased trunk fat mass, has an increased lean body mass, has a decreased trunk fat mass as a percentage of total fat mass, has normalized IGF-1 levels, or a combination thereof.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of about 1 mg/week to about 5 mg/week; ii) taking a measurement of an IGF-1 level in the subject at day 3 to day 4 after administering the long-acting rhGH, wherein the IGF-1 level in the subject has an SDS of >+1.5; and iii) administering the long-acting rhGH to the subject at a modified dose level, wherein the modified dose level is about 0.5 mg/week lower than the initial dose level if the IGF-1 level in the subject has an SDS of >+1.5. In some embodiments, the long-acting rhGH is administered once a week at an initial dose level or a modified dose level.

In some embodiments, the initial dose is 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of about 1 mg/week to about 5 mg/week; ii) taking a measurement of an IGF-1 level in the subject at day 3 to day 4 after administering the long-acting rhGH, wherein the IGF-1 level in the subject has an SDS of >+1.5; and iii) administering the long-acting rhGH to the subject at a modified dose level, wherein the modified dose level is about 0.75 mg/week lower than the initial dose level if the IGF-1 level in the subject has an SDS of >+1.5. In some embodiments, the long-acting rhGH is administered once a week at an initial dose level or a modified dose level. In some embodiments, the initial dose is 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of about 1 mg/week to about 5 mg/week; ii) taking a measurement of an IGF-1 level in the subject at day 3 to day 4 after administering the long-acting rhGH, wherein the IGF-1 level in the subject has an SDS of <−0.5; and iii) administering the long-acting rhGH to the subject at a modified dose level, wherein the modified dose level is about 1.0 mg/week higher than the initial dose level if the IGF-1 level in the subject has an SDS of <−0.5. In some embodiments, the long-acting rhGH is administered once a week at an initial dose level or a modified dose level. In some embodiments, the initial dose is 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of about 1 mg/week to about 5 mg/week; ii) taking a measurement of an IGF-1 level in the subject at day 3 to day 4 after administering the long-acting rhGH, wherein the IGF-1 level in the subject has an SDS of <−0.5; and iii) administering the long-acting rhGH to the subject at a modified dose level, wherein the modified dose level is about 1.5 mg/week higher than the initial dose level if the IGF-1 level in the subject has an SDS of <−0.5. In some embodiments, the long-acting rhGH is administered once a week at an initial dose level or a modified dose level. In some embodiments, the initial dose is 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose of about 1 mg/week to about 5 mg/week; ii) taking a measurement of an IGF-1 level in the subject at day 3 to day 4 after administering the long-acting rhGH, wherein the IGF-1 level in the subject has an SDS of >+1.5; and iii) administering the long-acting rhGH to the subject at a modified dose level, wherein the modified dose level is about 0.5 mg/week or about 0.75 mg/week lower than the initial dose level if the IGF-1 level in the subject has an SDS of >+1.5; taking an additional at least one measurement of the IGF-1 level in the subject after administering the modified dose level, wherein the IGF-1 level after administering the modified dose level has an SDS of >+1.5; and administering the long-acting rhGH to the subject at a further modified dose level, wherein the further modified dose level is about 0.5 mg/week lower than the modified dose level if the IGF-1 level in the subject has an SDS of >+1.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of >+1.5; and iii) administering the long-acting rhGH to the subject at a modified dose level, wherein the modified dose level is about 0.5 mg/week or about 0.75 mg/week lower than the initial dose level if the IGF-1 level in the subject has an SDS of >+1.5; taking an additional at least one measurement of the IGF-1 level in the subject after administering the modified dose level, wherein the IGF-1 level after administering the modified dose level has an SDS of >+1.5; and administering the long-acting rhGH to the subject at a further modified dose level, wherein the further modified dose level is about 0.75 mg/week lower than the modified dose level if the IGF-1 level in the subject has an SDS of >+1.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of >+1.5; and iii) administering the long-acting rhGH to the subject at a modified dose level, wherein the modified dose level is about 0.5 mg/week or about 0.75 mg/week lower than the initial dose level if the IGF-1 level in the subject has an SDS of >+1.5; taking an additional at least one measurement of the IGF-1 level in the subject after administering the modified dose level, wherein the IGF-1 level after administering the modified dose level has an SDS of <−0.5; and administering the long-acting rhGH to the subject at a further modified dose level, wherein the further modified dose level is about 1.0 mg/week higher than the modified dose level if the IGF-1 level in the subject has an SDS of <−0.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of >+1.5; and iii) administering the long-acting rhGH to the subject at a modified dose level, wherein the modified dose level is about 0.5 mg/week or about 0.75 mg/week lower than the initial dose level if the IGF-1 level in the subject has an SDS of >+1.5; taking an additional at least one measurement of the IGF-1 level in the subject after administering the modified dose level, wherein the IGF-1 level after administering the modified dose level has an SDS of <−0.5; and administering the long-acting rhGH to the subject at a further modified dose level, wherein the further modified dose level is about 1.5 mg/week higher than the modified dose level if the IGF-1 level in the subject has an SDS of <−0.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of <−0.5; and iii) administering the long-acting rhGH to the subject at a modified dose level, wherein the modified dose level is about 1.0 mg/week or about 1.5 mg/week higher than the initial dose level if the IGF-1 level in the subject has an SDS of <−0.5; taking an additional at least one measurement of the IGF-1 level in the subject after administering the modified dose level, wherein the IGF-1 level after administering the modified dose level has an SDS of >+1.5; and administering the long-acting rhGH to the subject at a further modified dose level, wherein the further modified dose level is about 0.5 mg/week lower than the modified dose level if the IGF-1 level in the subject has an SDS of >+1.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of <−0.5; and iii) administering the long-acting rhGH to the subject at a modified dose level, wherein the modified dose level is about 1.0 mg/week or about 1.5 mg/week higher than the initial dose level if the IGF-1 level in the subject has an SDS of <−0.5; taking an additional at least one measurement of the IGF-1 level in the subject after administering the modified dose level, wherein the IGF-1 level after administering the modified dose level has an SDS of >+1.5; and administering the long-acting rhGH to the subject at a further modified dose level, wherein the further modified dose level is about 0.75 mg/week lower than the modified dose level if the IGF-1 level in the subject has an SDS of >+1.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of <−0.5; and iii) administering the long-acting rhGH to the subject at a modified dose level, wherein the modified dose level is about 1.0 mg/week or about 1.5 mg/week higher than the initial dose level if the IGF-1 level in the subject has an SDS of <−0.5; taking an additional at least one measurement of the IGF-1 level in the subject after administering the modified dose level, wherein the IGF-1 level after administering the modified dose level has an SDS of <−0.5; and administering the long-acting rhGH to the subject at a further modified dose level, wherein the further modified dose level is about 1.0 mg/week higher than the modified dose level if the IGF-1 level in the subject has an SDS of <−0.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of <−0.5; and iii) administering the long-acting rhGH to the subject at a modified dose level, wherein the modified dose level is about 1.0 mg/week or about 1.5 mg/week higher than the initial dose level if the IGF-1 level in the subject has an SDS of <−0.5; taking an additional at least one measurement of the IGF-1 level in the subject after administering the modified dose level, wherein the IGF-1 level after administering the modified dose level has an SDS of <−0.5; and administering the long-acting rhGH to the subject at a further modified dose level, wherein the further modified dose level is about 1.5 mg/week higher than the modified dose level if the IGF-1 level in the subject has an SDS of <−0.5. In some embodiments, the long-acting rhGH is administered once a week at an initial dose level, at a modified dose level, or at a further modified dose level.

In some embodiments, a method of treating growth hormone deficiency in an adult subject in need thereof comprises: i) administering a long-acting recombinant human growth hormone (rhGH) comprising the amino acid sequence of SEQ ID NO:2 to the subject at an initial dose level of about 1 mg/week to about 5 mg/week; ii) taking a measurement of an IGF-1 level in the subject at day 3 to day 4 after administering the long-acting rhGH, wherein the IGF-1 level in the subject has an SDS of <−0.5 or >+1.5; iii) administering the long-acting rhGH to the subject at a modified dose level, wherein the modified dose level is about 0.5 mg/week or about 0.75 mg/week lower than the initial dose level if the IGF-1 level in the subject has an SDS level of >+1.5 or wherein the modified dose level is about 1.0 mg/week or about 1.5 mg/week higher than the initial dose level if the IGF-1 level in the subject has an SDS level of <−0.5; iv) taking a measurement of an IGF-1 level in the subject after administering the modified dose level; and v) administering the long-acting rhGH to the subject at a further modified dose level, wherein the further modified dose level is about 0.5 mg/week or about 0.75 mg/week lower than the modified dose level if the IGF-1 level in the subject has an SDS level of >+1.5 or wherein the further modified dose level is about 1.0 mg/week or about 1.5 mg/week higher than the modified dose level if the IGF-1 level in the subject has an SDS level of <−0.5. In some embodiments, the long-acting rhGH is administered once a week at an initial dose level, at a modified dose level or at a further modified dose level. In some embodiments, an IGF-1 level is measured in serum or plasma.

In other embodiments, a defined range is a therapeutic dose range achieved by administering a long-acting rhGH provided herein. In other embodiments, a defined range is one in which the Cₘₐₓ and C_{trough} of the sinusoidal behavior of IGF-1 are maintained following consecutive administrations of a long-acting rhGH provided herein. In other embodiments, a defined range is a therapeutic dose range for consecutively administering a long-acting rhGH provided herein with excellent responsiveness in a subject and with minimal need for dose modification. In other embodiments, a defined range is comparable to the range of IGF-1 levels in individuals that are considered to be normal. In other embodiments, a defined range is the normal range of IGF-1 levels/values in normal individuals. In another yet embodiment, the defined range is within the normal range when IGF-1 SDS values are within ±2 SDS.

In other embodiments, a long-acting rhGH described herein is used in the same manner as unmodified growth hormones. In other embodiments, a long-acting rhGH described herein has an increased circulating half-life and plasma residence time, decreased clearance, and increased clinical activity in vivo. In other embodiments, due to the improved properties of a long-acting rhGH described herein, these conjugates are administered less frequently than unmodified growth hormones. In other embodiments, a long-acting rhGH as described herein is administered once a week to once every two weeks. In other embodiments, a long-acting rhGH as described herein is administered once every two weeks to once every three weeks. In other embodiments, decreased frequency of administration will result in improved patient compliance leading to improved treatment outcomes, as well as improved patient quality of life. In other embodiments, compared to conventional conjugates of growth hormones linked to poly(ethylene glycol) it has been found that growth hormone CTP conjugates having the molecular weight and linker structure of the conjugates of this invention have an improved potency, improved stability, elevated AUC levels, enhanced circulating half-life. In other embodiments, compared to conventional conjugates of growth hormones linked to poly(ethylene glycol) it has been found that growth hormones having the molecular weight and linker structure of the conjugates of this invention have an improved potency, improved stability, elevated AUC levels, enhanced circulating half-life. In other embodiments, a therapeutically effective amount of a conjugated growth hormone is the amount of conjugate necessary for the in vivo measurable expected biological activity. In other embodiments, a growth hormone utilized according to the teachings disclosed herein exhibits increased potency. In some embodiments, attachment of CTP sequence to both the amino and carboxy termini of a growth hormone results in prolonged in-vivo activity.

In other embodiments, a therapeutically effective amount of a long-acting rhGH described herein is determined according to factors as the exact type of condition being treated, the condition of the patient being treated, as well as the other ingredients in the composition. In other embodiments, a therapeutically effective amount of a conjugated growth hormone is about 0.1 mg to about 1 mg per kg body weight administered once a week. In other embodiments, a therapeutically effective amount of a conjugated growth hormone is about 0.5 mg to about 0.8 mg per kg body weight, administered once a week. In other embodiments, a therapeutically effective amount of a conjugated growth hormone is about 0.6 mg to about 0.7 mg per kg body weight, administered once a week. In other embodiments, a therapeutically effective amount of a conjugated growth hormone is about 0.66 mg per kg body weight, administered once a week. In other embodiments, a therapeutically effective amount of a conjugated growth hormone is 0.66 mg per kg body weight, administered subcutaneously once a week. In other embodiments, a pharmaceutical composition comprising a conjugated growth hormone is formulated at strength effective for administration by various means to a human patient.

In other embodiments, the methods of the invention include increasing the compliance in the use of GH therapy, comprising providing to a subject in need thereof, a long-acting rhGH described herein, thereby increasing compliance in the use of growth hormone therapy.

In other embodiments, methods provided herein include increasing the compliance of subjects afflicted with chronic illnesses that are in need of a GH therapy. In other embodiments, methods of the invention enable reduction in the dosing frequency of a GH by modifying the GH with CTPs as described hereinabove. In other embodiments, the term compliance comprises adherence. In other embodiments, methods of the invention include increasing the compliance of patients in need of a GH therapy by reducing the frequency of administration of the GH. In other embodiments, reduction in the frequency of administration of the GH is achieved due to the CTP modifications which render the CTP-modified GH more stable. In other embodiments, reduction in frequency of administration of the GH is achieved as a result of increasing half-life of the growth hormone. In other embodiments, reduction in frequency of administration of a GH is achieved as a result of increasing clearance time of the GH. In other embodiments, reduction in the frequency of administration of a growth hormone is achieved as a result of increasing the AUC measure of the growth hormone.

In other embodiments, the present teachings provide a method of decreasing body fat in a non-human subject, comprising administering to said subject a therapeutically effective amount of an expression vector comprising a polynucleotide, said polynucleotide consisting of a non-human growth hormone, one chorionic gonadotropin carboxy terminal peptide (CTP) attached to the amino terminus of said non-human growth hormone, and two chorionic gonadotropin CTPs attached to the carboxy terminus of said non-human growth hormone, and wherein said polypeptide optionally consists of a signal peptide attached to the amino terminus of said one CTP, thereby inducing growth or weight gain in a non-human subject.

In other embodiments, the present teachings provide a method of increasing insulin-like growth factor (IGF-1) levels in a human subject, comprising administering to said subject a therapeutically effective amount of a polypeptide comprising a growth hormone, one chorionic gonadotropin carboxy terminal peptide (CTP) attached to the amino terminus of said growth hormone, and two chorionic gonadotropin CTPs attached to the carboxy terminus of said growth hormone, thereby increasing IGF-1 levels in said subject.

In other embodiments, the present teachings provide a method of increasing insulin-like growth factor (IGF-1) levels in a non-human subject, comprising administering to said subject a therapeutically effective amount of an expression vector comprising a polynucleotide, said polynucleotide consisting of a non-human growth hormone, one chorionic gonadotropin carboxy terminal peptide (CTP) attached to the amino terminus of said non-human growth hormone, and two chorionic gonadotropin CTPs attached to the carboxy terminus of said non-human growth hormone, and wherein said polypeptide optionally consists of a signal peptide attached to the amino terminus of said one CTP, thereby inducing growth or weight gain in a non-human subject.

In other embodiments, the present teachings provide a method of improving the area under the curve (AUC) of a growth hormone in a subject, comprising administering to said subject a therapeutically effective amount of a long-acting rhGH, thereby reducing the dosing frequency of a growth hormone in a subject.

In other embodiments, the methods provide a long-acting rhGH for stimulating muscle growth. In some embodiments, increasing IGF-1 levels in a human subject may be effective in treating, preventing or suppressing type 1 diabetes, type 2 diabetes, amyotrophic lateral sclerosis (ALS aka “Lou Gehrig's Disease”), severe burn injury and myotonic muscular dystrophy (MMD).

In other embodiments, the methods utilize any of the long-acting rhGH described herein for stimulating bone growth. In such embodiments, the bone growth of the treated patient correlates with the chronological age of said patient. Thus, in some embodiments the present invention is directed to a method of treatment that provides efficacious bone maturation rates.

In other embodiments, the methods provide a nucleic acid sequence encoding a long-acting rhGH described herein, for stimulating bone growth.

In other embodiments, the methods provide a nucleic acid sequence encoding long-acting rhGH as described herein. In other embodiments, the methods provide a nucleic acid sequence encoding a long-acting rhGH for stimulating muscle growth, increasing cardiac function, stimulating bone growth, maintaining muscle integrity, balancing muscle metabolism, inducing muscle buildup, inducing de-novo muscle build-up, enhancing bone load, treating symptoms associated with osteoporosis, treating a wasting disease, increasing lipolysis, improving fluid balance, treating osteoporosis, improving lung function, improving immunity, regrowing a vital organ, increasing sense of well-being, restoring REM sleep, or any combination thereof.

In other embodiments, a nucleic acid molecule encoding a growth hormone as described herein encodes any amino acid sequence of a growth hormone known to one of skill in the art.

In other embodiments, the methods provide a long-acting rhGH for the treatment of wasting disease, AIDS, cachexia, or hGH deficiency.

In some embodiments, the methods provided herein are employed in veterinary medicine. In some embodiments, the present teachings provide treatment of domesticated mammals which are maintained as human companions (e.g., dogs, cats, horses), which have significant commercial value (e.g., dairy cows, beef cattle, sporting animals), which have significant scientific value (e.g., captive or free specimens of endangered species), or which otherwise have value.

In other embodiments, the present teachings provide a method of inducing growth or weight gain in a human subject, comprising the step of administering to said human subject a therapeutically effective amount of an expression vector comprising a polynucleotide consisting of a nucleic acid encoding a long-acting rhGH comprising human growth hormone, one chorionic gonadotropin CTP attached to the amino terminus of said non-human growth hormone, and two chorionic gonadotropin CTPs attached to the carboxy terminus of the human growth hormone, thereby inducing growth or weight gain in the human subject.

In other embodiments, the present teachings provide a method of inducing weight loss or decreasing body fat in a subject, comprising administering to said subject a long-acting rhGH provided herein, thereby inducing weight loss or decreasing body fat in said subject. In some embodiments, the present teachings provide a method of decreasing trunk fat mass, increasing lean body mass, decreasing trunk fat mass as a percentage of total fat mass, normalizing IGF-1 levels, or a combination thereof, in a subject (e.g., an adult). In some embodiments, said subject is obese. In other embodiments, said subject is overweight.

In other embodiments, the present teachings provide a method of decreasing body fat in a non-human subject, comprising administering to said subject a long-acting rhGH provided herein, thereby inducing growth or weight gain in a non-human subject.

In other embodiments, the present teachings provide a method of decreasing fat deposits in a subject. In other embodiments, the present teachings provide a method of increasing muscle mass in a subject. In other embodiments, the present teachings provide a method of promoting muscle growth in a subject. In other embodiments, the present teachings provide a method of increasing muscle to fat ratio. In other embodiments, the present teachings provide a method of decreasing body mass index (BMI) or Quetelet index.

In other embodiments, growth is measured by weight gain. In other embodiments, growth is measured by height gain. In other embodiments, growth is measured by weight gain. In other embodiments, growth is measured by muscle mass gain. In other embodiments, growth is measured by weight gain. In other embodiments, growth is measured by bone mass gain. In other embodiments, growth is measured by weight gain. In other embodiments, growth is measured by muscle mass gain. In other embodiments, the weight gain is due to bone and/or muscle mass gain. In other embodiments, growth is measured by any known measure known to one of skill in the art.

In some embodiments, human growth hormone polypeptides can be used to treat a subject, with conditions related to growth and weight, such as a growth deficiency disorder, AIDS wasting, aging, impaired immune function of HIV-infected subjects, a catabolic illness, surgical recovery, a congestive cardiomyopathy, liver transplantation, liver regeneration after hepatectomy, chronic renal failure, renal osteodystrophy, osteoporosis, achondroplasia/hypochondroplasia, skeletal dysplasia, a chronic inflammatory or nutritional disorder such as Crohn's disease, short bowel syndrome, juvenile chronic arthritis, cystic fibrosis, male infertility, X-linked hypophosphatemic rickets, Down's syndrome, Spina bifida, Noona Syndrome, obesity, impaired muscle strength and fibromyalgia. In some embodiments, interferon polypeptides are used to treat a subject, with a variety of conditions such as hairy cell leukemia (HCL), Kaposi's sarcoma (KS), chronic myelogenous leukemia (CML), chronic Hepatitis C (CHC), condylomata acuminata (CA), chronic Hepatitis B, malignant melanoma, follicular non-Hodgkin's lymphoma, multiple sclerosis, chronic granulomatous disease, Mycobacterium avium complex (MAC), pulmonary fibrosis and osteoporosis.

In some embodiments, the polypeptides can be provided to the individual per se. In some embodiments, the polypeptides can be provided to the individual as part of a pharmaceutical composition where it is mixed with a pharmaceutically acceptable carrier.

Method of Making

In some embodiments, the present teachings provide a process for making a long-acting rhGH pharmaceutical composition for a once a week administration to a subject having a growth hormone deficiency, the process comprising the steps of:
a. modifying a growth hormone by attaching one chorionic gonadotropin carboxy terminal peptide (CTP) attached to the amino terminus of said growth hormone, and two chorionic gonadotropin CTPs attached to the carboxy terminus of said growth hormone;
b. mixing the modified growth hormone in step a. with a buffer and a tonicity agent; and,
c. pre-filling a pen or syringe with the mixture produced in step b.

In some embodiments, the present teachings provide a process for filling a pen or syringe with a formulation provided herein comprising the steps of:
a. formulating a once a week dosage form of said long-acting rhGH having a pre-determined amount of long-acting rhGH; and,
b. filling the pen or syringe with the formulation.

In some embodiments, the present teachings provide pharmaceutical formulations comprising a long-acting rhGH comprising: a growth hormone, a single chorionic gonadotropin carboxy terminal peptide attached to the amino terminus of the growth hormone, and two chorionic gonadotropin carboxy terminal peptides attached to the carboxy terminus of the growth hormone. In other embodiments, the present teachings provide a pharmaceutical formulation comprising a long-acting rhGH comprising a growth hormone, a single chorionic gonadotropin carboxy terminal peptide attached to the amino terminus of the growth hormone, two chorionic gonadotropin carboxy terminal peptides attached to the carboxy terminus of the growth hormone, and a signal peptide attached to the amino terminus of one chorionic gonadotropin carboxy terminal peptide. In other embodiments, a pharmaceutical formulation further comprises a buffer and a tonicity agent. In some embodiments, a buffer is 10 mM citrate and the tonicity agent is 147 mM NaCl. In some embodiments, a formulation is at about a pH of 6.6. In other embodiments, a formulation is at about a pH of 6.5. In other embodiments, a formulation is at about a pH of 6.4. In some embodiments, a buffer is 10 mM citrate, a tonicity agent is 147 mM NaCl, and the pH is about 6.6. In other embodiments, a formulation is at about a pH range of 6.0-6.8.

In some embodiments, a formulation is a liquid formulation.

In some embodiments, the present teachings provide a formulation comprising a long-acting rhGH, wherein said formulation has increased stability. In some embodiments, a formulation is stable for at least one year. In other embodiments, a formulation is stable for at least two years.

In other embodiments, provided herein are a once weekly dosage forms comprising pharmaceutical compositions and pharmaceutical formulations provided herein.

In some embodiments, any of the compositions provided herein may comprise at least two CTP sequences bound to a protein of interest, in any form. In some embodiments, the present teachings provide combined preparations. In some embodiments, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In some embodiments, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In some embodiments, “excipient” refers to an inert substance added to a pharmaceutical composition to further facilitate administration of an active ingredient. In some embodiments, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, PA, latest edition, which is incorporated herein by reference.

In some embodiments, the long-acting rhGH provided herein is subcutaneously administered to a subject. In some embodiments, the preparation is administered in a local rather than systemic manner, for example, via injection of the preparation directly into a specific region of a patient's body. In other embodiments, the long-acting rhGH is injected below the skin (subcutaneous injection). In other embodiments, the long-acting rhGH is injected below the skin. In other embodiments, the long-acting rhGH is injected into the muscle. In other embodiments, the long-acting rhGH is injected into the muscle (intramuscular injection). In other embodiments, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In some embodiments, where the pharmaceutical composition or the pharmaceutical composition is administered via injection to a subject, it is done so using a prefilled syringe or a pen.

The pharmaceutical compositions provided herein can be subcutaneously administered to a subject using one or more of several modes of administration, including, but not limited to, syringes, pens, pumps, or any combination thereof. For example, single-use syringes be used to administer discrete bolus injections of the compositions. Syringes useful for administrations of the compositions provided herein include, for example without limitation, syringes which can be designed to hold about 1 ml, about 1.1 ml, about 1.2 ml, about 1.3 ml, about 1.4 ml, about 1.5 ml, about 1.6 ml, about 1.7 ml, about 1.8 ml, about 1.9 ml, or about 2 ml, and have markings in units for ease of administration.

In other embodiments, the pharmaceutical compositions are administered by intravenous, intra-arterial, or intramuscular injection of a liquid preparation. In some embodiments, liquid formulations include solutions, suspensions, dispersions, emulsions, oils and the like. In some embodiments, the pharmaceutical compositions are administered intravenously, and are thus formulated in a form suitable for intravenous administration. In other embodiments, the pharmaceutical compositions are administered intra-arterially, and are thus formulated in a form suitable for intra-arterial administration. In other embodiments, the pharmaceutical compositions are administered intramuscularly, and are thus formulated in a form suitable for intramuscular administration.

In other embodiments, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In some embodiments, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In some embodiments, formulation is dependent upon the route of administration chosen.

In some embodiments, injectables, of the invention are formulated in aqueous solutions. In some embodiments, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, the preparation of the present invention is formulated in liquid formulations for subcutaneous injection via a prefilled syringe or pen. In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In some embodiments, depending on the severity and responsiveness of the condition to be treated, dosing can be of a single or a plurality of administrations, with course of treatment lasting from several weeks to several years or until cure is effected or diminution of the disease state is achieved.

In some embodiments, the amount of a composition or formulation to be administered will, of course, be dependent on the subject being treated, the severity of the affliction, the manner of administration, the judgment of the prescribing physician, etc.

In some embodiments, compositions including the preparation of the present invention formulated in a compatible pharmaceutical carrier are also be prepared, placed in an appropriate container, and labeled for treatment of an indicated condition.

In other embodiments, the pharmaceutical composition comprising a long-acting rhGH as described herein is further formulated to comprise complex carriers such as human serum albumin, polyols, sugars, and anionic surface active stabilizing agents. See, for example, WO 89/10756 (Hara et al.—containing polyol and p-hydroxybenzoate). In other embodiments, the pharmaceutical composition comprises a growth hormone as described herein and is further formulated to comprise lactobionic acid and an acetate/glycine buffer. In other embodiments, the pharmaceutical composition comprising a long-acting rhGH as described herein is further formulated to comprise amino acids, such as arginine or glutamate that increase the solubility of interferon compositions in water. In other embodiments, the pharmaceutical composition comprises a long-acting rhGH as described herein and is further formulated to comprise glycine or human serum albumin (HSA), a buffer (e g. acetate) and an isotonic agent (e.g., NaCl). In other embodiments, the pharmaceutical composition comprises a long-acting rhGH as described herein and is further formulated to comprise a phosphate buffer, glycine and HSA.

In other embodiments, the pharmaceutical composition comprising a long-acting rhGH provided herein is formulated in a liquid composition comprising a stabilizing agent at between about 0.3% and 5% by weight which is an amino acid.

In other embodiments, the pharmaceutical composition comprising a long-acting rhGH provided herein provides dosing accuracy and product safety. In other embodiments, the pharmaceutical composition comprising a long-acting rhGH provided herein provides a biologically active, stable liquid formulation for use in injectable applications. In other embodiments, the pharmaceutical composition comprises a non-lyophilized long-acting rhGH provided herein.

In other embodiments, the pharmaceutical composition comprising a long-acting rhGH as described herein provides a liquid formulation permitting storage for a long period of time in a liquid state facilitating storage and shipping prior to administration.

In other embodiments, the pharmaceutical composition comprising a long-acting rhGH as described herein comprises solid lipids as matrix material. In other embodiments, the injectable pharmaceutical composition comprising a long-acting rhGH as described herein comprises solid lipids as matrix material. In other embodiments, the production of lipid microparticles by spray congealing was described by Speiser (Speiser and al., Pharm. Res. 8 (1991) 47-54) followed by lipid nanopellets for peroral administration (Speiser EP 0167825 (1990)). In other embodiments, lipids, which are used, are well tolerated by the body (e. g. glycerides composed of fatty acids which are present in the emulsions for parenteral nutrition). In other embodiments, the pharmaceutical composition comprising a long-acting rhGH as described herein is in the form of liposomes (J. E. Diederichs and al., Pharm./nd. 56 (1994) 267-275).

In some embodiments, it will be appreciated that the long-acting rhGH can be provided to the individual with additional active agents to achieve an improved therapeutic effect as compared to treatment with each agent by itself. In other embodiments, measures (e.g., dosing and selection of the complementary agent) are taken to minimize adverse side effects which are associated with combination therapies.

Additional objects, advantages, and novel features of the present invention will become apparent to one ordinarily skilled in the art upon examination of the following embodiments and examples, which are not intended to be limiting. Additionally, each of the various embodiments of the present invention as delineated hereinabove and as claimed in the claims section below finds experimental support in the following examples.

### EMBODIMENTS

Embodiment 1. A method of treating growth hormone deficiency in a subject in need thereof, the method comprising: administering a long-acting recombinant human growth hormone (rhGH) comprising the amino acid sequence of SEQ ID NO:2 to the subject at an initial dose level; taking at least two measurements of an IGF-1 level in the subject, wherein the IGF-1 level in the subject on two consecutive measurements taken 4 to 6 weeks apart each has a standard deviation score (SDS) of >+2; and administering the long-acting rhGH to the subject at a modified dose level wherein the modified dose level is about 15% lower than the initial dose level.

Embodiment 2. The method of embodiment 1, wherein the long-acting rhGH is administered once a week at the initial dose level or at the modified dose level.

Embodiment 3. The method of embodiment 1 or 2, wherein the subject is a pediatric subject.

Embodiment 4. The method of any one of embodiments 1 to 3, wherein the initial dose level is about 0.66 mg per kg of body weight per week.

Embodiment 5. The method of any one of embodiments 1 to 4, wherein the at least two IGF-1 level measurements are taken at day 3 to day 4 (e.g., about 96 hours) after administering the long-acting rhGH at an initial dose level.

Embodiment 6. The method of any one of embodiments 1 to 5, wherein the modified dose level is about 0.56 mg per kg body weight per week.

Embodiment 7. The method of any one of embodiments 1 to 6, further comprising taking at least one measurement of an IGF-1 level in the subject at least 4 weeks after administering the modified dose level.

Embodiment 8. The method of embodiment 7, wherein the at least one measurement of an IGF-1 level in the subject has an SDS of >+2.

Embodiment 9. The method of embodiment 8, further comprising administering the long-acting rhGH to the subject at a further modified dose level.

Embodiment 10. The method of embodiment 9, wherein the long-acting rhGH is administered once a week at the further modified dose level.

Embodiment 11. The method of embodiment 9 or 10, wherein the further modified dose level is 15% lower than the modified dose level.

Embodiment 12. The method of any one of embodiments 9 to 11, wherein the further modified dose level is about 0.48 mg per kg body weight per week.

Embodiment 13. A method of treating growth hormone deficiency in a subject in need thereof, the method comprising: administering a long-acting recombinant human growth hormone (rhGH) comprising the amino acid sequence of SEQ ID NO:2 to the subject at an initial dose level of about 0.66 mg per kg body weight per week; taking at least two measurements of an IGF-1 level in the subject at day 3 to day 4 after administering the long-acting rhGH, wherein the IGF-1 level in the subject on two consecutive measurements taken 4 to 6 weeks apart each has a standard deviation score (SDS) of >+2; administering the long-acting rhGH to the subject at a modified dose level, wherein the modified dose level is 15% lower than the initial dose level; taking at least one measurement of an IGF-1 level in the subject at least 4 weeks after administering the modified dose level, wherein the IGF-1 level at least 4 weeks after administering the modified dose level has a SDS of >+2; and administering the long-acting rhGH to the subject at a further modified dose level, wherein the further modified dose level is 15% lower than the modified dose level.

Embodiment 14. The method of embodiment 13, wherein the long-acting rhGH is administered once a week at an initial dose level, at a modified dose level or at a further modified dose level.

Embodiment 15. The method of embodiment 13 or 14, wherein the subject is a pediatric subject.

Embodiment 16. The method of any one of embodiments 13 to 15, wherein the IGF-1 level is measured in serum or plasma.

Embodiment 17. The method of any one of embodiments 13 to 16, wherein the modified dose level is about 0.56 mg per kg body weight per week.

Embodiment 18. The method of any one of embodiments 13 to 17, wherein the further modified dose level is about 0.48 mg per kg body weight per week.

Embodiment 19. Use of a long-acting recombinant human growth hormone (rhGH) for the treatment of a growth hormone deficiency (GHD) in a subject in need thereof, the treatment comprising administering the amino acid sequence of SEQ ID NO:2 to the subject at an initial dose level; taking at least two measurements of an IGF-1 level in the subject, wherein the IGF-1 level in the subject on two consecutive measurements taken 4 to 6 weeks apart each has a standard deviation score (SDS) of >+2; and administering the long-acting rhGH to the subject at a modified dose level wherein the modified dose level is about 15% lower than the initial dose level.

Embodiment 20. The use of embodiment 19, wherein the long-acting rhGH is administered once a week at the initial dose level or at the modified dose level.

Embodiment 21. The use of embodiment 19 or 20, wherein the subject is a pediatric subject.

Embodiment 22. The use of any one of embodiments 19 to 21, wherein the initial dose level is about 0.66 mg per kg of body weight per week.

Embodiment 23. The use of any one of embodiments 19 to 22, wherein the at least two IGF-1 level measurements are taken at day 3 to day 4 (e.g., about 96 hours) after administering the long-acting rhGH at an initial dose level.

Embodiment 24. The use of any one of embodiments 19 to 23, wherein the modified dose level is about 0.56 mg per kg body weight per week.

Embodiment 25. The use of any one of embodiments 19 to 24, wherein the treatment further comprises taking at least one measurement of an IGF-1 level in the subject at least 4 weeks after administering the modified dose level.

Embodiment 26. The use of embodiment 25, wherein the at least one measurement of an IGF-1 level in the subject has an SDS of >+2.

Embodiment 27. The use of embodiment 26, wherein the treatment further comprises administering the long-acting rhGH to the subject at a further modified dose level.

Embodiment 28. The use of embodiment 27, wherein the long-acting rhGH is administered once a week at the further modified dose level.

Embodiment 29. The use of embodiment 27 or 28, wherein the further modified dose level is 15% lower than the modified dose level.

Embodiment 30. The use of any one of embodiments 27 to 29, wherein the further modified dose level is about 0.48 mg per kg body weight per week.

Embodiment 31. Use of a long-acting recombinant human growth hormone (rhGH) for the treatment of a growth hormone deficiency (GHD) in a subject in need thereof, the treatment comprising administering a long-acting recombinant human growth hormone (rhGH) comprising the amino acid sequence of SEQ ID NO:2 to the subject at an initial dose level of about 0.66 mg per kg body weight per week; taking at least two measurements of an IGF-1 level in the subject at day 3 to day 4 after administering the long-acting rhGH, wherein the IGF-1 level in the subject on two consecutive measurements taken 4 to 6 weeks apart each has a standard deviation score (SDS) of >+2; administering the long-acting rhGH to the subject at a modified dose level, wherein the modified dose level is 15% lower than the initial dose level; taking at least one measurement of an IGF-1 level in the subject at least 4 weeks after administering the modified dose level, wherein the IGF-1 level at least 4 weeks after administering the modified dose level has a SDS of >+2; and administering the long-acting rhGH to the subject at a further modified dose level, wherein the further modified dose level is 15% lower than the modified dose level.

Embodiment 32. The use of embodiment 31, wherein the long-acting rhGH is administered once a week at an initial dose level, at a modified dose level or at a further modified dose level.

Embodiment 33. The use of embodiment 31 or 32, wherein the subject is a pediatric subject.

Embodiment 34. The use of any one of embodiments 31 to 33, wherein the IGF-1 level is measured in serum or plasma.

Embodiment 35. The use of any one of embodiments 31 to 34, wherein the modified dose level is about 0.56 mg per kg body weight per week.

Embodiment 36. The use of any one of embodiments 31 to 35, wherein the further modified dose level is about 0.48 mg per kg body weight per week.

Embodiment 37. A method of treating growth hormone deficiency in an adult subject in need thereof, the method comprising: administering a long-acting recombinant human growth hormone (rhGH) comprising the amino acid sequence of SEQ ID NO:2 to the subject at an initial dose level; taking at least one measurement of an IGF-1 level in the subject wherein the IGF-1 level in the subject has a standard deviation score (SDS) of >+1.5 or <−0.5; and administering the long-acting rhGH to the subject at a modified dose level, wherein the modified dose level is about 0.5 mg/week or about 0.75 mg/week lower than the initial dose level when the IGF-1 level is the subject has an SDS value of >+1.5, or wherein the modified dose level is about 1.0 mg/week or about 1.5 mg/week higher than the initial dose level when the IGF-1 level in the subject has an SDS of <−0.5.

Embodiment 38. The method of embodiment 37, wherein the long-acting rhGH is administered once a week at the initial dose level or at the modified dose level.

Embodiment 39. The method of embodiment 37 or 38, wherein the initial dose level ranges from about 1 mg/week to about 5 mg/week.

Embodiment 40. The method of any one of embodiments 37 to 39, wherein the subject is a male, a female not on oral estrogen or a female on oral estrogen.

Embodiment 41. The method of embodiment 40, wherein the initial dose is: about 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

Embodiment 42. The method of any one of embodiments 37 to 41, wherein the IGF-1 level is measured in serum or plasma.

Embodiment 43. The method of any one of embodiments 37 to 42, wherein the IGF-1 level is measured at day 3 to day 4 after administering the long-acting rhGH at an initial dose level.

Embodiment 44. The method of any one of embodiments 40 to 43, wherein the modified dose level is about 0.5 mg/week lower than the initial dose level when the IGF-1 level in a male or a female not on oral estrogen has an SDS of >+1.5.

Embodiment 45. The method of any one of embodiments 40 to 43, wherein the modified dose level is about 0.75 mg/week lower than the initial dose level when the IGF-1 level in a female on oral estrogen has an SDS of >+1.5.

Embodiment 46. The method of any one of embodiments 40 to 45, wherein the modified dose level is: about 2.0 mg/week for a male 50 years of age or less, about 1.5 mg/week for a male greater than 50 years of age, about 2.5 mg/week for a female not on oral estrogen who is 50 years of age or less, about 2.0 mg/week for a female not on oral estrogen who is greater than 50 years of age, about 3.25 mg/week for a female on oral estrogen who is 50 years of age or less or about 2.75 mg/week for a female on oral estrogen who is greater than 50 years of age when the IGF-1 level in the male or female has an SDS of >+1.5.

Embodiment 47. The method of any one of embodiments 40 to 43, wherein the modified dose level is about 1.0 mg/week higher than the initial dose level when the IGF-1 level in a male or a female not on oral estrogen has an SDS of <−0.5.

Embodiment 48. The method of any one of embodiments 40 to 43, wherein the modified dose level is about 1.5 mg/week higher than the initial dose level when the IGF-1 level in a female on oral estrogen has an SDS of <−0.5.

Embodiment 49. The method of any one of embodiments 40 to 43, 47, and 48, wherein the modified dose is: about 3.5 mg/week for a male 50 years of age or less, about 3.0 mg/week for a male greater than 50 years of age, about 4.0 mg/week for a female not on oral estrogen who is 50 years of age or less, about 3.5 mg/week for a female not on oral estrogen who is greater than 50 years of age, about 5.5 mg/week for a female on oral estrogen who is 50 years of age or less or about 5.0 mg/week for a female on oral estrogen who is greater than 50 years of age when the IGF-1 level in the male or female has an SDS of <−0.5.

Embodiment 50. The method of any one of embodiments 19 to 49, further comprising taking a measurement of an IGF-1 level in a subject after administering the long-acting rhGH at the modified dose level.

Embodiment 51. The method of embodiment 50, wherein the IGF-1 level in the subject has an SDS of >+1.5.

Embodiment 52. The method of embodiment 51, further comprising administering the long-acting rhGH to the subject at a further modified dose level.

Embodiment 53. The method of embodiment 52, wherein the further modified dose level is about 0.5 mg/week lower than the modified dose level if the subject is a male or if the subject is a female who is not on estrogen.

Embodiment 54. The method of embodiment 52, wherein the further modified dose level is about 0.75 mg/week lower than the modified dose level if the subject is a female on estrogen.

Embodiment 55. The method of embodiment 50, wherein the IGF-1 level is the subject has an SDS of <−0.5.

Embodiment 56. The method of embodiment 55, further comprising administering the long-acting rhGH to the subject at a further modified dose level.

Embodiment 57. The method of embodiment 56, wherein the further modified dose level is about 1.0 mg/week higher than the modified dose level if the subject is a male or a female not on estrogen.

Embodiment 58. The method of embodiment 56, wherein the further modified dose level is about 1.5 mg/week higher than the modified dose level if the subject is a female on estrogen.

Embodiment 59. The method of any one of embodiments 52 to 58, further comprising administering the long-acting rhGH one, two, three, four, five, six, seven, eight, nine, ten or more times, taking a measurement of the IGF-1 level in the subject at day 3 to day 4 after each administration and reducing the dose level of long-acting rhGH by about 0.5 mg/week or about 0.75 mg/week based on age, gender and estrogen status if the IGF-1 level has an SDS value of >+1.5 or increasing the dose level of long-acting rhGH by about 1.0 mg/week or about 1.5 mg/week based on age, gender and estrogen status if the IGF-1 level has an SDS of <−0.5.

Embodiment 59. The method of any one of embodiments 37 to 58, wherein the subject's trunk fat mass is decreased, lean body mass is increased, trunk fat mass as a percentage of total fat mass is decreased, IGF-1 levels are normalized, or a combination thereof.

Embodiment 60. A method of treating growth hormone deficiency (GHD) in an adult subject in need thereof, the method comprising: administering a long-acting recombinant human growth hormone (rhGH) comprising the amino acid sequence of SEQ ID NO:2 to the subject at an initial dose level of about 1 mg/week to about 5 mg/week; taking at least one measurement of an IGF-1 level in the subject at day 3 to day 4 after administering the long-acting rhGH, wherein the IGF-1 level in the subject has an SDS of <−0.5 or >+1.5; administering the long-acting rhGH to the subject at a modified dose level, wherein the modified dose level is about 0.5 mg/week or about 0.75 mg/week lower than the initial dose level if the IGF-1 level in the subject has an SDS of >+1.5 or wherein the modified dose level is about 1.0 mg/week or about 1.5 mg/week higher than the initial dose level if the IGF-1 level in the subject has an SDS of <−0.5; optionally taking a measurement of the IGF-1 level in the subject after administering the modified dose level, wherein the IGF-1 level after administering the modified dose level has an SDS of <−0.5 or >+1.5; and administering the long-acting rhGH to the subject at a further modified dose level, wherein the further modified dose level is about 0.5 mg/week or about 0.75 mg/week lower than the modified dose level if the IGF-1 level in the subject has an SDS of >+1.5 or wherein the further modified dose level is about 1.0 mg/week or about 1.5 mg/week higher than the modified dose level if the IGF-1 level in the subject has an SDS of <−0.5.

Embodiment 61. The method of embodiment 60, wherein the long-acting rhGH is administered once a week at an initial dose level, at a modified dose level or at a further modified dose level.

Embodiment 62. The method of embodiment 60 or 61, wherein the IGF-1 level is measured in serum or plasma.

Embodiment 63. Use of a long-acting recombinant human growth hormone (rhGH) for the treatment of a growth hormone deficiency (GHD) in a subject in need thereof, the treatment comprising administering a long-acting recombinant human growth hormone (rhGH) comprising the amino acid sequence of SEQ ID NO:2 to the subject at an initial dose level; taking at least one measurement of an IGF-1 level in the subject wherein the IGF-1 level in the subject has a standard deviation score (SDS) of >+1.5 or <−0.5; and administering the long-acting rhGH to the subject at a modified dose level, wherein the modified dose level is about 0.5 mg/week or to about 0.75 mg/week lower than the initial dose level when the IGF-1 level is the subject has an SDS value of >+1.5, or wherein the modified dose level is about 1.0 mg/week or about 1.5 mg/week higher than the initial dose level when the IGF-1 level in the subject has an SDS of <−0.5.

Embodiment 64. The use of embodiment 63, wherein the long-acting rhGH is administered once a week at the initial dose level or at the modified dose level.

Embodiment 65. The use of embodiment 63 or 64, wherein the initial dose level ranges from about 1 mg/week to about 5 mg/week.

Embodiment 66. The use of any one of embodiments 63 to 65, wherein the subject is a male, a female not on oral estrogen or a female on oral estrogen.

Embodiment 67. The use of embodiment 66, wherein the initial dose is: about 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less or about 3.5 mg/week for a female on oral estrogen who is greater than 50 years of age.

Embodiment 68. The use of any one of embodiments 63 to 67, wherein the IGF-1 level is measured in serum or plasma.

Embodiment 69. The use of any one of embodiments 63 to 68, wherein the IGF-1 level is measured at day 3 to day 4 after administering the long-acting rhGH at an initial dose level.

Embodiment 70. The use of any one of embodiments 66 to 69, wherein the modified dose level is about 0.5 mg/week lower than the initial dose level when the IGF-1 level in a male or a female not on oral estrogen has an SDS of >+1.5.

Embodiment 71. The use of any one of embodiments 66 to 69, wherein the modified dose level is about 0.75 mg/week lower than the initial dose level when the IGF-1 level in a female on oral estrogen has an SDS of >+1.5.

Embodiment 72. The use of any one of embodiments 66 to 71, wherein the modified dose level is: about 2.0 mg/week for a male 50 years of age or less, about 1.5 mg/week for a male greater than 50 years of age, about 2.5 mg/week for a female not on oral estrogen who is 50 years of age or less, about 2.0 mg/week for a female not on oral estrogen who is greater than 50 years of age, about 3.25 mg/week for a female on oral estrogen who is 50 years of age or less or about 2.75 mg/week for a female on oral estrogen who is greater than 50 years of age when the IGF-1 level in the male or female has an SDS of >+1.5.

Embodiment 73. The use of any one of embodiments 66 to 69, wherein the modified dose level is about 1.0 mg/week higher than the initial dose level when the IGF-1 level in a male or a female not on oral estrogen has an SDS of <−0.5.

Embodiment 74. The use of any one of embodiments 66 to 69, wherein the modified dose level is about 1.5 mg/week higher than the initial dose level when the IGF-1 level in a female on oral estrogen has an SDS of <−0.5.

Embodiment 75. The use of any one of embodiments 66 to 69, 73, and 74, wherein the modified dose is: about 3.5 mg/week for a male 50 years of age or less, about 3.0 mg/week for a male greater than 50 years of age, about 4.0 mg/week for a female not on oral estrogen who is 50 years of age or less, about 3.5 mg/week for a female not on oral estrogen who is greater than 50 years of age, about 5.5 mg/week for a female on oral estrogen who is 50 years of age or less or about 5.0 mg/week for a female on oral estrogen who is greater than 50 years of age when the IGF-1 level in the male or female has an SDS of <−0.5.

Embodiment 76. The use of any one of embodiments 63 to 75, wherein the treatment further comprises taking a measurement of an IGF-1 level in a subject after administering the long-acting rhGH at the modified dose level.

Embodiment 77. The use of embodiment 76, wherein the IGF-1 level in the subject has an SDS of >+1.5.

Embodiment 78. The use of embodiment 77, wherein the treatment further comprises administering the long-acting rhGH to the subject at a further modified dose level.

Embodiment 79. The use of embodiment 78, wherein the further modified dose level is about 0.5 mg/week lower than the modified dose level if the subject is a male or if the subject is a female who is not on estrogen.

Embodiment 80. The use of embodiment 78, wherein the further modified dose level is about 0.75 mg/week lower than the modified dose level if the subject is a female on estrogen.

Embodiment 81. The use of embodiment 76, wherein the IGF-1 level is the subject has an SDS of <−0.5.

Embodiment 82. The use of embodiment 81, wherein the treatment further comprises administering the long-acting rhGH to the subject at a further modified dose level.

Embodiment 83. The use of embodiment 82, wherein the further modified dose level is about 1.0 mg/week higher than the modified dose level if the subject is a male or a female not on estrogen.

Embodiment 84. The use of embodiment 82, wherein the further modified dose level is about 1.5 mg/week higher than the modified dose level if the subject is a female on estrogen.

Embodiment 85. The use of any one of embodiments 78 to 84, wherein the treatment further comprises administering the long-acting rhGH one, two, three, four, five, six, seven, eight, nine, ten or more times, taking a measurement of the IGF-1 level in the subject at day 3 to day 4 after each administration and reducing the dose level of long-acting rhGH by about 0.5 mg/week or about 0.75 mg/week based on age, gender and estrogen status if the IGF-1 level has an SDS value of >+1.5 or increasing the dose level of long-acting rhGH by about 1.0 mg/week or about 1.5 mg/week based on age, gender and estrogen status if the IGF-1 level has an SDS of <−0.5.

Embodiment 86. The use of any one of embodiments 63 to 85, wherein the subject's trunk fat mass is decreased, lean body mass is increased, trunk fat mass as a percentage of total fat mass is decreased, IGF-1 levels are normalized, or a combination thereof.

Embodiment 87. Use of a long-acting recombinant human growth hormone (rhGH) for the treatment of a growth hormone deficiency (GHD) in a subject in need thereof, the treatment comprising administering a long-acting recombinant human growth hormone (rhGH) comprising the amino acid sequence of SEQ ID NO:2 to the subject at an initial dose level of about 1 mg/week to about 5 mg/week; taking at least one measurement of an IGF-1 level in the subject at day 3 to day 4 after administering the long-acting rhGH, wherein the IGF-1 level in the subject has an SDS of <−0.5 or >+1.5; administering the long-acting rhGH to the subject at a modified dose level, wherein the modified dose level is about 0.5 mg/week or about 0.75 mg/week lower than the initial dose level if the IGF-1 level in the subject has an SDS of >+1.5 or wherein the modified dose level is about 1.0 mg/week or about 1.5 mg/week higher than the initial dose level if the IGF-1 level in the subject has an SDS of <−0.5; optionally taking a measurement of the IGF-1 level in the subject after administering the modified dose level, wherein the IGF-1 level after administering the modified dose level has an SDS of <−0.5 or >+1.5; and administering the long-acting rhGH to the subject at a further modified dose level, wherein the further modified dose level is about 0.5 mg/week or about 0.75 mg/week lower than the modified dose level if the IGF-1 level in the subject has an SDS of >+1.5 or wherein the further modified dose level is about 1.0 mg/week or about 1.5 mg/week higher than the modified dose level if the IGF-1 level in the subject has an SDS of <−0.5.

Embodiment 88. The use of embodiment 87, wherein the long-acting rhGH is administered once a week at an initial dose level, at a modified dose level or at a further modified dose level.

Embodiment 89. The use of embodiment 87 or 88, wherein the IGF-1 level is measured in serum or plasma.

Embodiment 90. A method of treating growth hormone deficiency in an adult subject in need thereof, the method comprising: administering a long-acting recombinant human growth hormone (rhGH) comprising the amino acid sequence of SEQ ID NO:2 to the subject at an initial dose level; monitoring the subject for an adverse event; and administering the long-acting rhGH to the subject at a modified dose level, wherein the modified dose level is 25% lower than the initial dose level if the adverse event is moderate, or wherein the modified dose level is 50% lower than the initial dose level if the adverse event is severe.

Embodiment 91. The method of embodiment 90, wherein the adverse event is edema, hypertension, carpal tunnel, glucose, or a combination thereof. Embodiment 92. Use of a long-acting recombinant human growth hormone (rhGH) for the treatment of a growth hormone deficiency (GHD) in a subject in need thereof, the treatment comprising administering a long-acting recombinant human growth hormone (rhGH) comprising the amino acid sequence of SEQ ID NO:2 to the subject at an initial dose level; monitoring the subject for an adverse event; and administering the long-acting rhGH to the subject at a modified dose level, wherein the modified dose level is 25% lower than the initial dose level if the adverse event is moderate, or wherein the modified dose level is 50% lower than the initial dose level if the adverse event is severe.

Embodiment 93. The use of embodiment 92, wherein the adverse event is edema, hypertension, carpal tunnel, glucose, or a combination thereof. Embodiment 94. A method of treating growth hormone deficiency in a first subject in need thereof, the method comprising: selecting a first subject with growth hormone deficiency, wherein the first subject has previously received a once daily recombinant human growth hormone (once daily rhGH) therapy; and administering an effective amount of a long-acting recombinant human growth hormone (long-acting rhGH) to the first subject, so that efficacy of the long-acting rhGH in the first subject is comparable to efficacy of the long-acting rhGH in a second subject who has previously received only the long-acting rhGH and has not previously received the once daily rhGH therapy.

Embodiment 95. The method of embodiment 94, wherein the long-acting rhGH is a C-terminal peptide (CTP)-modified hGH.

Embodiment 96. The method of embodiment 94 or 95, wherein the long-acting rhGH comprises the amino acid sequence of mature human growth hormone (hGH) with one copy of CTP from the beta chain of human chorionic gonadotropin at the hGH N-terminus and two copies of CTP in tandem at the hGH C-terminus.

Embodiment 97. The method of any one of embodiments 94 to 96, wherein the long-acting rhGH comprises the amino acid sequence shown in SEQ ID NO: 2.

Embodiment 98. The method of any one of embodiments 94 to 97, wherein the long-acting rhGH is glycosylated.

Embodiment 99. The method of any one of embodiments 94 to 98, wherein the long-acting rhGH is O-glycosylated on twelve to twenty serines.

Embodiment 100. The method of any one of embodiments 94 to 99, wherein the once daily rhGH is somatropin, somatrem, a somatropin biosimilar, or a somatrem biosimilar.

Embodiment 101. The method of any one of embodiments 94 to 100, wherein efficacy is assessed by measuring one or more of: mean height velocity, gain in height standard deviation score (SDS), body mass index, bone maturation, insulin growth factor-1 (IGF-1) SDS, insulin-like growth factor binding protein 3 IGFBP-3 SDS, pubertal status changed from Tanner 1, mean glucose, HbA1c, thyroid function, and cholesterol values.

Embodiment 102. The method of embodiment 101, wherein efficacy is indicated by continued bone maturation.

Embodiment 103. The method of any one of embodiments 94 to 100, wherein the long-acting rhGH is administered according to a dosage regimen comprising subcutaneous administration of 0.66 mg per kg body weight once weekly at any time of day.

Embodiment 104. The method of embodiment 103, wherein the long-acting rhGH is administered on the same day each week.

Embodiment 105. The method of embodiment 103, wherein the time between two doses is at least three days.

Embodiment 106. The method of any one of embodiments 94 to 105, wherein the once daily rhGH therapy is administered at a dosage of 0.16 to 0.24 mg per kg body weight per week.

Embodiment 107. The method of any one of embodiments 94 to 106, wherein the subject does not have active malignancy.

Embodiment 108. The method of any one of embodiments 94 to 107, wherein the subject does not have active malignancy, does not have an acute illness (complications following open heart or abdominal surgery, multiple accidental trauma, or acute respiratory failure).

Embodiment 109. The method of any one of embodiments 94 to 108, wherein the subject had received a once daily recombinant human growth hormone for at least three months.

Embodiment 110. The method of any one of embodiments 94 to 109, wherein the subject had received a once daily recombinant human growth hormone for at least six months.

Embodiment 111. The method of any one of embodiments 94 to 110, wherein the subject is obese.

Embodiment 112. The method of any one of embodiments 94 to 111, wherein the subject is a female.

Embodiment 113. The method of any one of embodiments 94 to 112, wherein the subject is 10 to 15 years old.

Embodiment 114. The method of any one of embodiments 94 to 113, wherein the subject has one or more of the following: isolated growth hormone deficiency (GHD), GH insufficiency as part of multiple pituitary hormone deficiency, pediatric GHD, and Prader-Willi Syndrome.

Embodiment 115. The method of any one of embodiments 94 to 114, wherein the subject has adult GHD.

Embodiment 116. The method of any one of embodiments 95 to 115, wherein the method further comprises monitoring glucose levels in the subject.

Embodiment 117. The method of any one of embodiments 95 to 116, wherein the long-acting rhGH is administered subcutaneously in the abdomen, thighs, buttocks, or upper arm.

Embodiment 118. The method of any one of embodiments 95 to 117, wherein the method demonstrates similar efficacy in a clinical study including participants divided into a test population and into a control population, wherein the test population receives (a) the once daily rhGH therapy for 12 months and then (b) the long-acting rhGH once weekly for 12 months, and the control population receives the long-acting rhGH once weekly for two years.

Embodiment 119. Use of a long-acting recombinant human growth hormone (long-acting rhGH) for the manufacture of a medicament for the treatment of growth hormone deficiency in a first subject in need thereof, wherein the use comprises administering an effective amount of the long-acting rhGH to a first subject with a growth hormone deficiency, wherein the first subject has previously received a once daily recombinant human growth hormone (once daily rhGH) therapy, and wherein efficacy of the long-acting rhGH in the first subject is comparable to efficacy of the long-acting rhGH in a second subject who has previously received only the long-acting rhGH and has not previously received the once daily rhGH therapy.

Embodiment 120. The use of embodiment 119, wherein the long-acting rhGH is a C-terminal peptide (CTP)-modified hGH.

Embodiment 121. The use of embodiment 119 or 120, wherein the long-acting rhGH comprises the amino acid sequence of mature human growth hormone (hGH) with one copy of CTP from the beta chain of human chorionic gonadotropin at the hGH N-terminus and two copies of CTP in tandem at the hGH C-terminus.

Embodiment 122. The use of any one of embodiments 119 to 121, wherein the long-acting rhGH comprises the amino acid sequence shown in SEQ ID NO: 2.

Embodiment 123. The use of any one of embodiments 119 to 122, wherein the long-acting rhGH is glycosylated.

Embodiment 124. The use of any one of embodiments 119 to 123, wherein the long-acting rhGH is O-glycosylated on twelve to twenty serines.

Embodiment 125. The use of any one of embodiments 119 to 124, wherein the once daily rhGH is somatropin, somatrem, a somatropin biosimilar, or a somatrem biosimilar.

Embodiment 126. The use of any one of embodiments 119 to 125, wherein efficacy is to assessed by measuring one or more of: mean height velocity, gain in height standard deviation score (SDS), body mass index, bone maturation, insulin growth factor-1 (IGF-1) SDS, insulin-like growth factor binding protein 3 IGFBP-3 SDS, pubertal status changed from Tanner 1, mean glucose, HbA1c, thyroid function, and cholesterol values.

Embodiment 127. The use of embodiment 126, wherein efficacy is indicated by continued bone maturation.

Embodiment 128. The use of any one of embodiments 119 to 127, wherein the long-acting rhGH is administered according to a dosage regimen comprising subcutaneous administration of 0.66 mg per kg body weight once weekly at any time of day.

Embodiment 129. The use of embodiment 128, wherein the long-acting rhGH is administered on the same day each week.

Embodiment 130. The use of embodiment 128, wherein the time between two doses is at least three days.

Embodiment 131. The use of any one of embodiments 119 to 130, wherein the once daily rhGH therapy is administered at a dosage of 0.16 to 0.24 mg per kg body weight per week.

Embodiment 132. The use of any one of embodiments 119 to 131, wherein the subject does not have active malignancy.

Embodiment 133. The use of any one of embodiments 119 to 132, wherein the subject does not have an acute illness selected from the group consisting of: complications following open heart or abdominal surgery, multiple accidental trauma, or acute respiratory failure.

Embodiment 134. The use of any one of embodiments 119 to 133, wherein the subject has one or more of the following: isolated growth hormone deficiency (GHD), GH insufficiency as part of multiple pituitary hormone deficiency, pediatric GHD, and Prader-Willi Syndrome.

Embodiment 135. The use of any one of embodiments 119 to 133, wherein the subject has adult GHD.

Embodiment 136. The use of any one of embodiments 119 to 135, wherein the use further comprises monitoring glucose levels in the subject.

Embodiment 137. The use of any one of embodiments 119 to 136, wherein the long-acting rhGH is administered subcutaneously in the abdomen, thighs, buttocks, or upper arm.

Embodiment 138. The use of any one of embodiments 119 to 137, wherein the use demonstrates similar efficacy in a clinical study including participants divided into a test population and into a control population, wherein the test population receives (a) the once daily rhGH therapy for 12 months and then (b) the long-acting rhGH once weekly for 12 months, and the control population receives the long-acting rhGH once weekly for two years.

Embodiment 139. A method of treating growth hormone deficiency in a subject in need to thereof, the method comprising: selecting a subject with growth hormone deficiency, wherein the subject has previously received a once daily recombinant human growth hormone (once daily rhGH) therapy; and administering an effective amount of a long-acting recombinant human growth hormone (long-acting rhGH) to the subject once weekly wherein the bone maturation rate of the subject previously on the once daily recombinant human growth hormone is comparable to the bone maturation rate of the subject while on the once daily recombinant treatment.

Embodiment 140. Use of a long-acting recombinant human growth hormone (rhGH) for the treatment of a growth hormone deficiency (GHD) in a subject in need thereof, the treatment comprising selecting a subject with growth hormone deficiency, wherein the subject has previously received a once daily recombinant human growth hormone (once daily rhGH) therapy; and administering an effective amount of a long-acting recombinant human growth hormone (long-acting rhGH) to the subject once weekly wherein the bone maturation rate of the subject previously on the once daily recombinant human growth hormone is comparable to the bone maturation rate of the subject while on the once daily recombinant treatment.

Embodiment 141. A method of treating growth hormone deficiency in a subject in need thereof, the method comprising: selecting a subject with growth hormone deficiency, wherein the subject has previously received a once daily recombinant human growth hormone (once daily rhGH) therapy; and administering an effective amount of a long-acting recombinant human growth hormone (long-acting rhGH) to the subject once weekly wherein one or more clinical measurements of efficacy of the subject previously on the once daily recombinant human growth hormone is comparable to one or more clinical measurements of efficacy of the subject while on the once daily recombinant treatment.

Embodiment 142. The method of embodiment 141, wherein one or more clinical measurements of efficacy is selected from the group consisting of mean height velocity, annual height velocity, gain in height standard deviation score (SDS), body mass index, bone maturation, insulin growth factor-1 (IGF-1) SDS, insulin-like growth factor binding protein 3 IGFBP-3 SDS, pubertal status changed from Tanner 1, mean glucose, HbA1c, thyroid function, cholesterol values and a combination thereof.

Embodiment 143. Use of a long-acting recombinant human growth hormone (rhGH) for the treatment of a growth hormone deficiency (GHD) in a subject in need thereof, the treatment comprising selecting a subject with growth hormone deficiency, wherein the subject has previously received a once daily recombinant human growth hormone (once daily rhGH) therapy; and administering an effective amount of a long-acting recombinant human growth hormone (long-acting rhGH) to the subject once weekly wherein one or more clinical measurements of efficacy of the subject previously on the once daily recombinant human growth hormone is comparable to one or more clinical measurements of efficacy of the subject while on the once daily recombinant treatment.

Embodiment 144. The use of embodiment 143, wherein one or more clinical measurements of efficacy is selected from the group consisting of mean height velocity, annual height velocity, gain in height standard deviation score (SDS), body mass index, bone maturation, insulin growth factor-1 (IGF-1) SDS, insulin-like growth factor binding protein 3 IGFBP-3 SDS, pubertal status changed from Tanner 1, mean glucose, HbA1c, thyroid function, cholesterol values and a combination thereof.

Embodiment 145. A plurality of long-acting recombinant human growth hormone (long-acting rhGH) molecules, wherein each long-acting rhGH molecule comprises the amino acid sequence of mature human growth hormone (hGH) with one copy of C-terminal peptide (CTP) from the beta chain of human chorionic gonadotropin at the hGH N-terminus and two copies of CTP in tandem at the hGH C-terminus, and wherein the plurality comprises about 9 to 20 O-glycans per intact long-acting rhGH molecule.

Embodiment 146. The plurality of embodiment 145, wherein each long-acting rhGH molecule comprises the amino acid sequence shown in SEQ ID NO: 2.

Embodiment 147. The plurality of embodiment 145 or 146, wherein the long-acting rhGH molecule are 0-glycosylated on twelve to twenty serines.

Embodiment 148. The plurality of any one of embodiments 145 to 147, wherein the plurality comprises a predominant glycoform having a molecular mass of about 40314 Da.

Embodiment 149. The plurality of embodiment 148, wherein the plurality comprises additional predominant 0-glycoforms having molecular masses of about 39657 and 40970 Da.

Embodiment 150. The plurality of any one of embodiments 145 to 147, wherein the plurality comprises about 10-19 O-glycans per intact long-acting rhGH molecule.

Embodiment 151. The plurality of any one of embodiments 145 to 147, wherein the plurality comprises sialylated and di-sialylated core-1 O-glycans.

Embodiment 152. The plurality of any one of embodiments 145 to 147, wherein each CTP region comprises 0-5 hydroxy additions per intact somatrogon molecule.

### EXAMPLES

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

### Example 1

An Open-Label Extension of a Clinical Trial of Once Weekly Somatrogon Compared to Daily Recombinant Human Growth Hormone in Pediatric Patients with Growth Hormone Deficiency

Objective: Assess the efficacy and safety of long-term exposure to somatrogon once weekly in pediatric subjects.

### Study Details

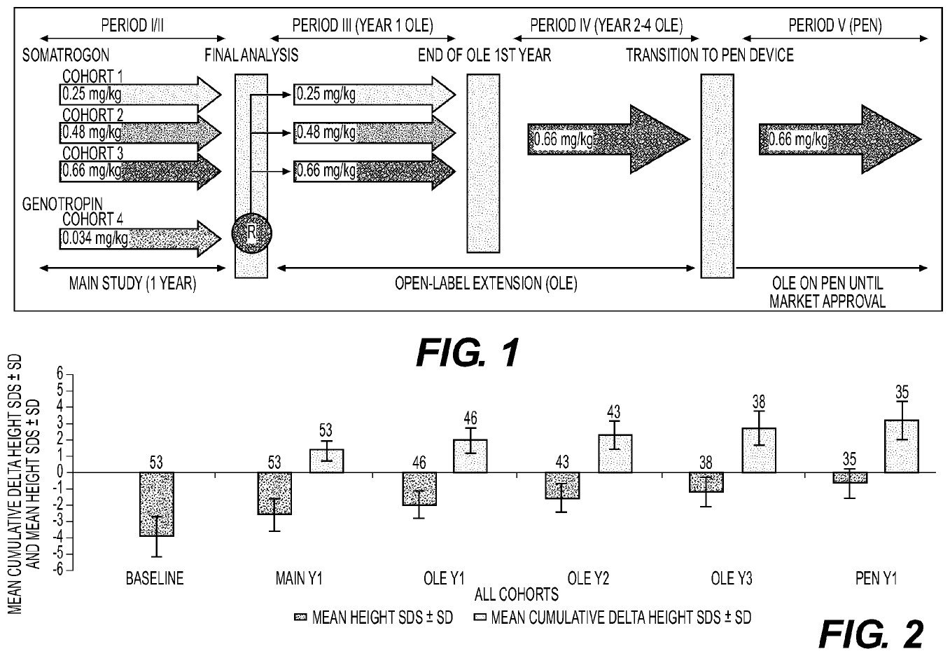

Somatrogon is a long-acting recombinant human growth hormone (rhGH) consisting of the amino acid sequence of human growth hormone and 3 copies of the carboxy-terminal peptide of human chorionic gonadotropin. Somatrogon is currently being developed as a once-weekly (QW) treatment for pediatric patients with growth hormone deficiency (GHD). This open-label extension (OLE) phase 2 study was a continuation of a randomized 12-month study that investigated the safety, efficacy, and tolerability of 3 dose levels of somatrogon QW (0.25, 0.48, or 0.66 mg/kg/wk) compared with once daily rhGH (Genotropin® 0.034 mg/kg/d) in initially rhGH-naïve prepubertal pediatric subjects with GHD. This global phase 2 study (NCT01592500) is comprised of 5 treatment periods (FIG. 1). The main study (Period I and II) found that subjects in all 3 somatrogon dose cohorts achieved adequate catch-up growth, with the highest dose cohort (0.66 mg/kg/wk) achieving the highest mean growth rate and an annualized height velocity (HV) closest to that of Genotropin® recipients. The OLE phase of the study (Periods III, IV, and V) followed patients for up to 5 additional years of exposure to somatrogon.

### Methods

Subjects who completed the main study (Periods I and II) and provided consent were eligible to be enrolled in the OLE study, which consisted of 3 periods (FIG. 1): Period III: an additional 12 months at the original somatrogon dose; Genotropin® recipients were randomized to 1 of the 3 somatrogon dose regimens. Period IV: Years 2-4 of the OLE, where all subjects received somatrogon at 0.66 mg/kg/wk. Period V: currently ongoing until marketing approval; subjects transitioned from single-use vials of somatrogon (subcutaneous injection via needle and syringe) to a prefilled pen device at the same somatrogon dose (0.66 mg/kg/wk).

Data up to 1 year of Period V are reported.

### Assessment and Endpoints

Annual height velocity (HV), change in height standard deviation score (SDS), and bone maturation were assessed every 12 months.

Safety evaluations included monitoring of all adverse events (AEs), including serious AEs and local injection site reactions, as well as laboratory assessments, including IGF-1 levels and immunogenicity.

Primary safety endpoints included the incidence of AEs and anti-drug antibody (ADA) formation, assessment of local site injections, IGF-1 levels, and IGF-1 SDS.

Secondary endpoints included annual HV, change in height SDS, and annual bone maturation.

All subjects were included in the full analysis set.

### Results

Study participants: 48 of 53 subjects who completed the main study were randomized and entered Period III of the OLE. At the start of Period III, the majority (66.7%) of subjects were male and almost all (93.8%) of the subjects were White (Table 1).

Completion rates for each OLE period (Periods III, IV, and Year 1 of Period V) ranged from 87.5 to 97.7%.

TABLE 1
Subject demographics and baseline characteristics
at the beginning of Period III of the OLE

| | Somatrogon Treatment Group^{a} | | | | | | | |
|---|---|---|---|---|---|---|---|---|
| | 0.25 | | 0.48 | | 0.66 | | | |
| | mg/kg/wk | | mg/kg/w | | mg/kg/wk | | Total | |
| n (%)^{b} | n = 16 | | n = 17 | | n = 15 | | N = 48 | |
| Age, mean (SD), y | 7.98 | (2.03) | 7.55 | (2.23) | 7.49 | (2.20) | 7.67 | (2.12) |
| Female | 3 | (18.8) | 6 | (35.3) | 7 | (46.7) | 16 | (33.3) |

Race

| | | | | | | | | |
|---|---|---|---|---|---|---|---|---|
| Black or | 0 | (0.0) | 1 | (5.9) | 0 | (0.0) | 1 | (2.1) |
| African American | | | | | | | | |
| White | 15 | (93.8) | 16 | (94.1) | 14 | (93.3) | 45 | (93.8) |
| Other | 1 | (6.3) | 0 | (0.0) | 1 | (6.7) | 2 | (4.2) |

Pubertal status

| | | | | | | | | |
|---|---|---|---|---|---|---|---|---|
| Tanner I | 16 | (100.0) | 17 | (100.0) | 14 | (93.3) | 47 | (97.9) |
| Tanner II | 0 | (0.0) | 0 | (0.0) | 1 | (6.7) | 1 | (2.1) |
| Tanner III | 0 | (0.0) | 0 | (0.0) | 0 | (0.0) | 0 | (0.0) |
| Tanner IV | 0 | (0.0) | 0 | (0.0) | 0 | (0.0) | 0 | (0.0) |

^{a}Includes subjects who received Genotropin ® during the main study and were re-randomized to receive somatrogon during the OLE period.
^{b}Except where indicated.
OLE = open-label extension;
SD = standard deviation

### Efficacy

At the end of Period III, the mean (SD) annual HV for the 0.25 and 0.48 mg/kg/wk dose cohorts was similar (7.73±1.89 and 7.54±1.28 cm/y, respectively), but was higher in the 0.66 mg/kg/wk dose cohort (8.81±1.12 cm/y), consistent with the results of the main study.

The HV in Periods IV and V indicated a sustained growth response that was independent of initial cohort assignment in the main study.

Relative to the main study baseline (−3.98±1.22), mean (SD) height SDS improved progressively throughout the OLE and was within the normal range (−0.69±0.87) at the end of the first year of Period V (FIG. 2).

Similar increases in mean annual bone maturation were observed across Periods III-V. Bone maturation with QW somatrogon did not advance discordantly relative to advancement in chronological age.

### Safety

Mean (SD) IGF-1 SDS values were similar at the end of Year 1 and 2 but increased at Year 3 (1.05±0.82) and at the end of the first year of Period V (1.29±0.81); mean IGF-1 SDS values were within the target therapeutic range and remained<2 SDS at all time points during the OLE.

During the OLE, 39 (81.3%) subjects reported≥1 treatment-emergent AE (TEAE) (Table 2); most TEAEs were mild or moderate in intensity and most were considered unrelated to study treatment.

All reported serious TEAEs (in 3 subjects) were considered unlikely related to study treatment, with the exception of 1 instance of scoliosis, which the investigator considered unexpected and probably related to study treatment.

During somatrogon administration with a needle and syringe (Period III and IV), no injection site reactions were reported; 3 (7.5%) subjects reported injection site reactions (bruising in 2 subjects and erythema in 1 subject) with the pen device, that were mild or moderate in intensity.

TABLE 2
Treatment-emergent adverse events observed
in ≥3 subjects (full analysis set)

| | All Subjects | | | | |
|---|---|---|---|---|---|
| | N = 48 | | | | |
| | Treatment- | Not related | | | |
| | related | to treatment | | Total | |
| Any adverse event | 7 (14.6) | 32 | (66.7) | 39 | (81.3) |
| Upper respiratory | 0 | 13 | (27.1) | 13 | (27.1) |
| tract infection | | | | | |
| Bronchitis | 0 | 11 | (22.9) | 11 | (22.9) |
| Nasopharyngitis | 0 | 6 | (12.5) | 6 | (12.5) |
| Rhinitis | 0 | 6 | (12.5) | 6 | (12.5) |
| Varicella | 0 | 5 | (10.4) | 5 | (10.4) |
| Ear infection | 0 | 4 | (8.3) | 4 | (8.3) |
| Pneumonia | 0 | 3 | (6.3) | 3 | (6.3) |
| Tonsilitis | 0 | 3 | (6.3) | 3 | (6.3) |
| Viral infections | 0 | 3 | (6.3) | 3 | (6.3) |
| Viral upper | 0 | 3 | (6.3) | 3 | (6.3) |
| respiratory tract | | | | | |
| infection | | | | | |
| Vomiting | 0 | 3 | (6.3) | 3 | (6.3) |
| Arthralgia | 0 | 3 | (6.3) | 3 | (6.3) |
| Pyrexia | 0 | 3 | (6.3) | 3 | (6.3) |
| Headache | 0 | 3 | (6.3) | 3 | (6.3) |
| Rhinitis allergic | 0 | 3 | (6.3) | 3 | (6.3) |

### Immunogenicity

ADAs were reported in 18 (37.5%) of 48 subjects during the OLE; 10 of these subjects also had ADAs in the main study.

No clinically meaningful differences in annual HV or TEAEs were observed between ADA-positive and ADA-negative subjects.

### Conclusions

Subjects treated with somatrogon once weekly for up to 5 years in the OLE study showed sustained improvement in annual HV, height SDS, and change in height SDS. Once weekly administration of somatrogon for an extended period was well tolerated in pediatric subjects with GHD.

### Example 2

Switch Data from the Open-Label Extension of the Pivotal Phase 3 Study of Once Weekly Somatrogon Compared to Daily Somatropin in Pediatric Patients with Growth Hormone Deficiency (pGHD)

Objectives: Compare the efficacy and safety of the soma/soma regimen (somatrogon administered once weekly in both the main study and the OLE) vs. the Geno/soma regimen (Genotropin® administered once daily in the main study and somatrogon administered once weekly in the OLE). This Example summarizes data from the first year of the optional open-label extension (OLE) of the pivotal phase 3 global trial (ClinicalTrials.gov: NCT02968004), comparing the efficacy and safety of children switched from Genotropin® (rhGH; somatropin) to Somatrogon (Geno/Soma) and children maintained on Somatrogon (Soma/Soma).

### Background

Somatrogon is a long-acting recombinant human growth hormone (rhGH) that comprises the amino acid sequence of human growth hormone and 3 copies of the carboxy-terminal peptide of human chorionic gonadotropin.

Somatrogon is in development as a once weekly (QW) treatment for children with growth hormone deficiency (GHD).

This open-label extension (OLE) phase 3 study was a continuation of a randomized 12-month main study (NCT02968004) that investigated the efficacy and safety of somatrogon, administered QW compared with rhGH (Genotropin®) administered once daily (QD) in initially rhGH-naïve prepubertal pediatric subjects with GHD (see Example 10).

The main study showed that QW somatrogon is noninferior to QD Genotropin® and that both treatments have a similar safety profile.

After completing the main study, subjects were then eligible to be consented and enrolled into the (optional) OLE study, in which all subjects received QW somatrogon.

### Methods

During the main study, 224 children were randomized to receive either once weekly somatrogon (0.66 mg/kg, n=109) or once daily Genotropin® (0.24 mg/kg/wk, n=115) for 12 months. Of these, 222 completed the 12 month main study, and 212 (95%) chose to enter the OLE study. By Sep. 30, 2020, 161 children (including 76 Geno/Soma) had complete mycological data at month 12 of the OLE.

The main study was an open-label, randomized, active controlled, parallel-group phase 3 study in which subjects were randomized 1:1 to receive QW subcutaneous (SC) doses of somatrogon (0.66 mg/kg/wk) or QD SC doses of Genotropin® (0.034 mg/kg/d) for 12 months.

Subjects who completed the main study and provided their consent were eligible to be enrolled in the single-arm OLE study.

Subjects who received somatrogon in the main study continued to receive somatrogon QW at the same dose (0.66 mg/kg/wk) (the “Soma/Soma” treatment group) while subjects who received Genotropin® in the main study were switched to somatrogon QW (0.66 mg/kg/wk) (the “Geno/Soma” treatment group).

Due to COVID-19, a number of subject visits were delayed for Month 12, resulting in lower than anticipated subject numbers for several parameters.

### Assessments and Endpoints

Clinical endpoints included annual height velocity (HV), change in height standard deviation score (SDS), and bone maturation, which were assessed every 12 months.

Biochemical endpoints included IGF-1 levels, IGF-1 SDS, IGFBP-3 levels, and IGFBP-3 SDS, which were assessed on Day 4 after somatrogon dosing across study visits.

### Results

Study participants: At the end of the main study, the least squares (LS) mean height velocity (HV) and the LS gain in height SDS for the somatrogon treatment group were 10.10 cm/year and 0.92; for the Genotropin® treatment group these were 9.78 cm/year and 0.87. Of the 222 subjects who completed the main study, 212 subjects entered the OLE. At the beginning of the OLE, demographics and baseline characteristics were well balanced between Soma/Soma and Geno/Soma treatment groups (Table 3). The majority of subjects were male (70.75%) and most subjects were White (76.42%). The Soma/Soma and Geno/Soma treatment groups had similar mean height SDS (−2.01 vs −1.94), mean BMI (16.95 vs 15.53 kg/m 2), and bone age (6.39 vs 6.37 y) at baseline (Table 4).

TABLE 3
Subject demographics and baseline characteristics
at the beginning of Period III of the OLE

| | Soma/Soma | | Geno/Soma | | | |
|---|---|---|---|---|---|---|
| | treatment | | treatment | | | |
| | group^{a} | | group^{b} | | Total | |
| | n = 104 | | n = 108 | | N = 212 | |
| Age, mean (SD), y | 8.89 | (2.67) | 8.69 | (2.37) | 8.79 | (2.52) |

Sex, n (%)

| | | | | | | |
|---|---|---|---|---|---|---|
| Male | 78 | (75.00) | 72 | (66.67) | 150 | (70.75) |
| Female | 26 | (25.00) | 36 | (33.33) | 62 | (29.25) |

Race, n (%)

| White | 79 | (75.96) | 83 | (76.85) | 162 | (76.42) |
|---|---|---|---|---|---|---|
| Black or | 0 | | 2 | (1.85) | 2 | (0.94) |
| African American | | | | | | |
| Asian | 21 | (20.19) | 17 | (15.74) | 38 | (17.92) |
| Other | 4 | (3.85) | 6 | (5.56) | 10 | (4.72) |
| Height, mean | 119.87 | (14.97) | 119.44 | (13.63) | 119.65 | (14.27) |
| (SD), cm | | | | | | |
| Weight, mean | 25.08 | (8.35) | 22.64 | (6.72) | 23.84 | (7.65) |
| (SD), kg | | | | | | |
| Target height | −0.88 | (0.95) | −0.68 | (1.01) | −0.78 | (0.98) |
| SDS, mean (SD) | | | | | | |

^{a}Subjects randomized to receive somatrogon in the main study.
^{b}Subjects randomized to receive Genotropin ® in the main study.
OLE = open-label extension;
SD = standard deviation

Efficacy: The mean (SD) height velocity (HV) at Month 12 of the OLE was 7.98 (1.81) cm/year for the Soma/Soma treatment group (Table 4). The mean (SD) HV at Month 12 of the OLE was 8.23 (1.88) cm/year for the Geno/Soma treatment group (Table 4). The mean (SD) change in height SDS from the beginning of the OLE to Month 12 was +0.42 (0.33) for the Soma/Soma treatment group and +0.49 (0.33) for the Geno/Soma treatment group (Table 4). The Soma/Soma and Geno/Soma treatment groups had similar mean height SDS (−1.46 vs −1.28), mean BMI (17.84 vs 17.58 kg/m 2), and mean bone age (8.29 vs 8.34 y) at Month 12 of the OLE (Table 4). An increase in bone maturation was observed from baseline to Month 12 for both the Soma/Soma and Geno/Soma treatment groups, indicating continued bone maturation (Table 4).

TABLE 4
Efficacy and safety at baseline and Month 12 of the OLE

| | Soma/Soma treatment group^{a} | | | | Geno/Soma treatment group^{b} | | | |
|---|---|---|---|---|---|---|---|---|
| | | | OLE | | | | OLE | |
| | OLE | | Month | | OLE | | Month | |
| Mean (SD)^{c} | Baseline | n | 12 | n | Baseline | n | 12 | n |
| Height | — | — | 7.98 | 84 | — | — | 8.23 | 78 |
| velocity, | | | (1.81) | | | | (1.88) | |
| cm/y | | | | | | | | |
| Height SDS | −2.01 | 103 | −1.46 | 84 | −1.94 | 108 | −1.28 | 78 |
| | (1.07) | | (0.87) | | (1.13) | | (0.78) | |
| BMI, kg/m² | 16.95 | 104 | 17.84 | 91 | 15.53 | 108 | 17.58 | 81 |
| | (2.29) | | (2.86) | | (1.73) | | (2.29) | |
| IGF-1 SDS | 0.63 | 102 | 1.14 | 82 | −0.70 | 105 | 1.28 | 76 |
| | (1.35) | | (1.22) | | (1.07) | | (1.16) | |
| IGFBP-3 | −0.05 | 103 | 0.27 | 83 | −0.71 | 106 | 0.41 | 76 |
| SDS | (0.86) | | (0.78) | | (1.00) | | (0.88) | |
| Bone age, y | 6.39 | 103 | 8.29 | 78 | 6.37 | 108 | 8.34 | 69 |
| | (2.76) | | (3.08) | | (2.68) | | (2.95) | |
| Bone | 0.70 | 103 | 0.80 | 78 | 0.71 | 108 | 0.82 | 69 |
| maturation | (0.17) | | (0.16) | | (0.17) | | (0.15) | |
| Change to | — | — | 1.78 | 68 | — | — | 1.64 | 66 |
| bone age | | | (0.84) | | | | (0.92) | |
| relative to | | | | | | | | |
| chronological | | | | | | | | |
| age (SD) | | | | | | | | |

Tanner stage, n (%)

| | | | | | | | | |
|---|---|---|---|---|---|---|---|---|
| Tanner I | 96 | 103 | 62 | 80 | 98 | 108 | 53 | 73 |
| | (93.20) | | (77.50) | | (90.74) | | (72.60) | |
| Tanner II | 5 | 103 | 13 | 80 | 9 | 108 | 12 | 73 |
| | (4.85) | | (16.25) | | (8.33) | | (16.44) | |
| Tanner III | 2 | 103 | 4 | 80 | 1 | 108 | 6 | 73 |
| | (1.94) | | (5.00) | | (0.93) | | (8.22) | |
| Tanner IV | — | — | 1 | 80 | — | — | 2 | 73 |
| | | | (1.25) | | | | (2.74) | |

^{a}Subjects randomized to receive somatrogon in the main study
^{b}Subjects randomized to receive Genotropin ® in the main study
^{c}unless otherwise stated
Mean (SD) time between dosing and IGF-1 sampling at OLE Month 12 was 3.54 (1.03) days for the Soma/Soma treatment group and 3.52 (1.21) days for the Geno/Soma treatment group

### Safety

Mean IGF-1 SDS values were higher at Month 12 vs baseline, for both the Soma/Soma (1.14 vs 0.63) and Geno/Soma (1.28 vs −0.70) treatment groups Dose reductions due to IGF-1 SDS>2 were required in 19 (18.3%) of 104 subjects in the soma/soma treatment group and 25 (23.1%) of 108 subjects in the Geno/Soma treatment group. Treatment-emergent adverse events (TEAEs) were reported in 71 (68.3%) and 87 (80.6%) subjects in the Soma/Soma and Geno/Soma treatment groups, respectively; most (≥90%) TEAEs were mild to moderate in severity. The Soma/Soma treatment group had no discontinuations due to TEAEs whereas 6 discontinuations occurred in the Geno/soma treatment group.

Across the 12 months of the OLE, mean glucose, HbA1c, FT4, TSH, and cholesterol (total, LDL, and HDL) values remained similar to baseline in both treatment groups.

### Conclusions

Height velocities and change in height SDS in the OLE were similar between the Geno/Soma and Soma/Soma treatment groups. The main phase of the global pivotal phase 3 trial demonstrated that Somatrogon (hGH-CTP) given once weekly is non-inferior to Genotropin® (hGH) while the OLE demonstrated that catch-up growth continued into the second year of treatment, with ‘switch’ from Genotropin® to somatrogon being non-inferior to somatrogon treatment only for two years. Metabolic (glycemic, lipid, and thyroid) parameters throughout the 12 months were similar to the levels observed at the OLE baseline, and levels were similar between treatment groups.

These results demonstrate that switching from Genotropin® administered once daily, to somatrogon administered weekly in the second year of the study was shown to be non-inferior to somatrogon given once weekly for the full two years. Catch-up growth continued, and metabolic parameters remained stable during the second year and were similar between groups, with no alarming safety signals.

### Example 3

Comparison of Quality of Life Responses from Caregiver and Children Aged ≥7 Years Using the Quality of Life in Short Stature Youth (QoLISSY) Questionnaire, Following 12 Months of Growth Hormone Treatment With Either a Weekly Somatrogon or a Daily Genotropin® Injection Schedule

Objectives: Evaluate the effect of weekly somatrogon and daily Genotropin® administration on QoL, as measured by the Quality of Life in Short Stature Youth (QoLISSY) during the first 12 months of treatment in a specific cohort of participants aged 7 years and older enrolled in a randomized controlled trial.

### Introduction

When focusing only on the data reported by children aged≥7 years, a comparable QoL improvement was demonstrated in this cohort of older children. This is one of the first clinical studies in which the QoLISSY has been administered to children with pGHD and their caregivers. Pediatric growth hormone deficiency (pGHD) is caused by a shortage of growth hormone (GH), leading to short stature. The prevalence is ˜1 in 4000 children 1-4; evidence suggest pGHD may be linked to lower emotional and social well-being. Treatment with daily subcutaneous injections of recombinant human GH (rhGH) increases height velocity (HV) and quality of life (QoL). New treatment approaches to enable once weekly dosing of rhGH are in development. Clinical trial NCT02968004 was a 12-month, open-label, multicenter, randomized control study to compare the efficacy and safety of once weekly somatrogon to daily Genotropin® in prepubertal children with pGHD. Inclusion and exclusion criteria are shown in Table 5. The study was powered for non-inferiority to evaluate the efficacy (i.e., HV after 12 months of treatment) and safety between the 2 treatments.

QoL was an exploratory endpoint evaluated using the validated Quality of Life in Short Stature Youth (QoLISSY) questionnaire, which assesses the impact of short stature on the QoL in children. The response scale is a 5-point Likert scale (“not at all/never” to “extremely/always”). Scores>70 indicate a good QoL.

TABLE 5
Inclusion and Exclusion Criteria

| Inclusion | Exclusion |
|---|---|
| ≥3 y old and <11 y old for girls | History of radiation therapy |
| (10 y and 364 d) or <12 y old for | or chemotherapy |
| boys (11 y and 364 d) | |
| Diagnosed with isolated GHD or GH | Children with psychosocial |
| insufficiency as part of multiple | dwarfism or born small for |
| pituitary hormone deficiency | gestational age |
| Peak plasma GH ≤10 ng/mL confirmed | Presence of anti-hGH Abs |
| by 2 tests | at screening |
| BA ≤ CA and not older than 10 y for | Any clinically significant |
| girls and 11 y for boys (9 and 10 y, | abnormality likely to affect |
| respectively, for CP-4-004) | growth or the ability to |
| | evaluate growth |
| No prior rhGH therapy | Closed epiphyses |
| Impaired HT and HV | |
| Baseline IGF-1 ≥ 1 SD below | |
| mean IGF-1 level standardized | |
| for age and sex (IGF-1 | |
| SDS ≤ −1.0) | |

Ab = antibody;
BA = bone age;
CA = chronologic age;
GH = growth hormone;
GHD = Growth Hormone Deficiency;
hGH = human growth hormone;
HT = height;
HV = height velocity;
IGF-1 = insulin-like growth factor-1;
rhGH = recombinant human growth hormone;
SD = standard deviation;
SDS = standard deviation score

### Methods

224 children participated in Study NCT02968004. Participants invited to complete the QoLISSY (appropriately translated) 10 were recruited from 8 countries: United States (n=41), Spain (n=19), UK (n=3), Belarus (n=2), Russia (n=20), Ukraine (n=24), Australia (n=4), and New Zealand (n=4). QoLISSY was administered to this cohort, at Baseline (BL) and 12 months after treatment start. QoLISSY-CHILD version was completed by children aged 7 years and older (somatrogon: n=35; Genotropin®: n=35). QoLISSY-PARENT version was intended to be completed by the caregiver for children<7 years of age (somatrogon: n=17; Genotropin®: n=26). However, the QoLISSY-PARENT was also completed by caregivers for a proportion of the children aged 7 years and older (somatrogon: n=26; Genotropin®: n=28). Reported in this study are QoLISSY results for children aged 7 years and older reported by either child or parent.

Normality was tested with the Shapiro-Wilkes test. With alpha at 0.05, statistical significance of between group differences in continuous outcomes was established using an unpaired t-test or the Mann-Whitney U test for normal or skewed data, respectively.

### Results

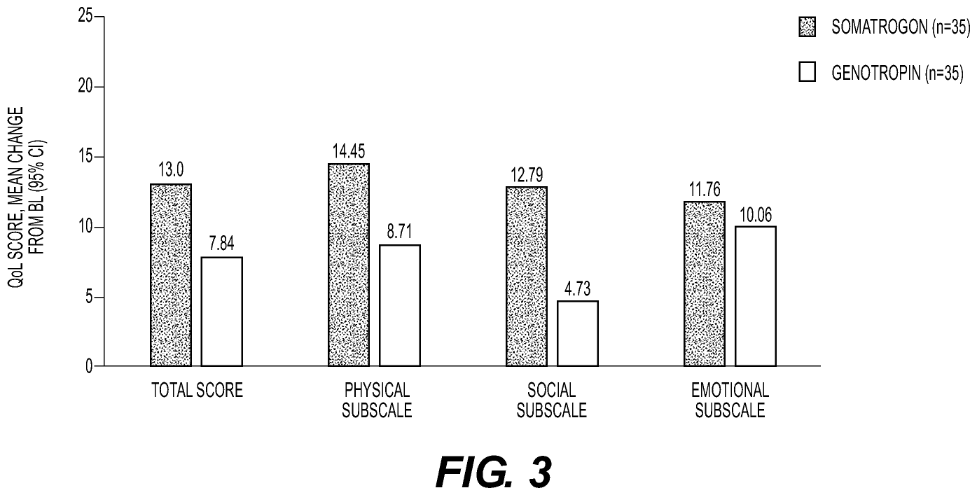

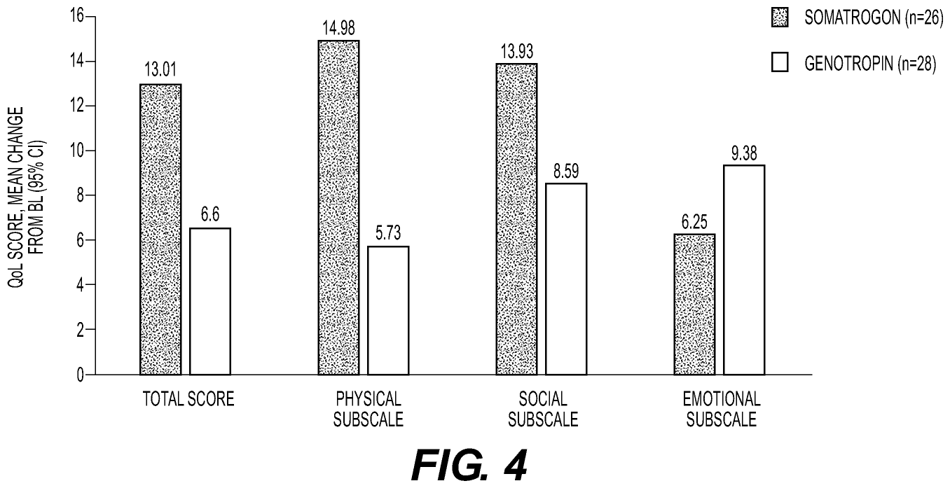

117 children/caregivers were invited to complete the QoLISSY. 70 children aged 7 years and older completed the QoLISSY-CHILD. 54 caregivers completed the QoLISSY-PARENT for children 7 years and older. Data from the QoLISSY-CHILD and QoLISSY-PARENT showed that both the somatrogon and Genotropin® treatment groups had increases in core total scores and subscale scores at 12 months, indicating similar improvements in QoL at 12 months for both weekly and daily treatments (FIG. 3 and FIG. 4).

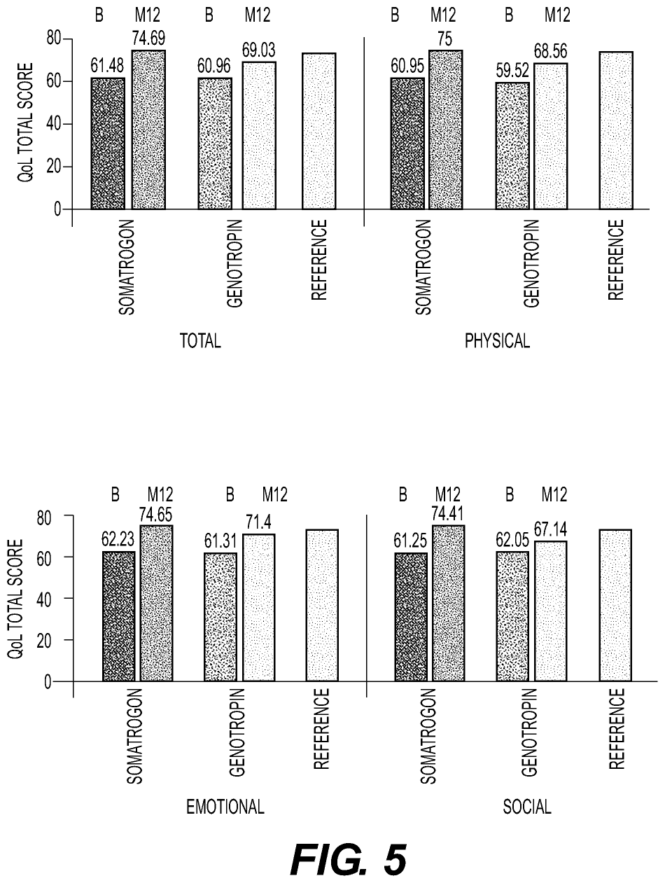

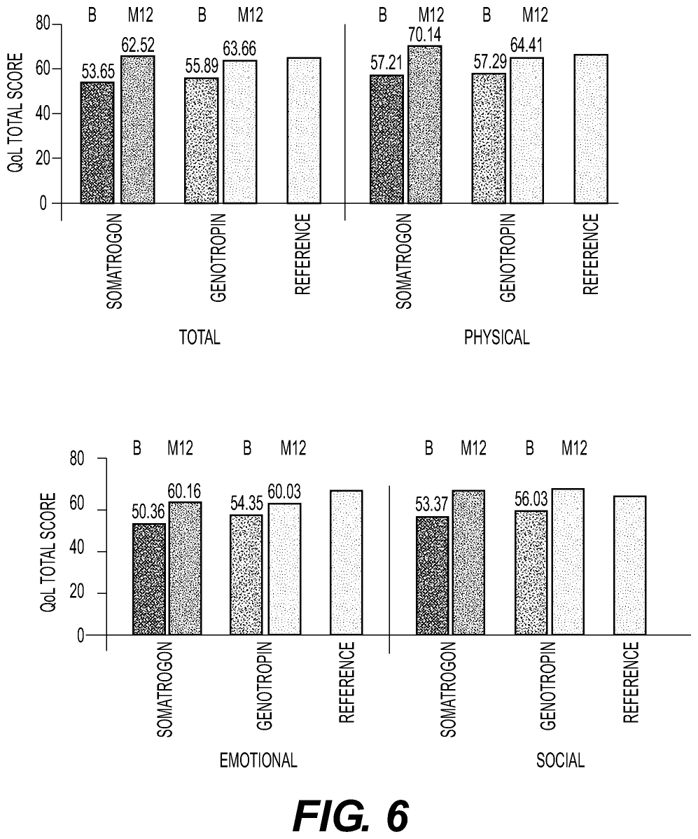

Numerically lower scores at BL and Month 12 for QoLISSY-PARENT were reported for this age group (≥7 years) compared with the BL scores and Month 12 reported in QoLISSY-CHILD (FIG. 5 and FIG. 6). Total QoLISSY-PARENT mean scores in the somatrogon group (n=26) were 53.65 (BL) and 65.52 (Month 12), with mean change of 13.01 (95% CI: 3.99, 22.02). In the Genotropin® group (n=28), mean scores were 55.89 (BL) and 63.66 (Month 12), with mean change of 6.60 (95% CI: −0.21, 13.40). Total QoLISSY-CHILD mean scores in the somatrogon group (n=35) were 61.48 (BL) and 74.69 (Month 12), with mean change of 13.00 (95% CI: 5.81, 20.19). In the Genotropin® group (n=35), mean scores were 60.96 (BL) and 69.03 (Month 12), with mean change of 7.84 (95% CI: 2.71, 12.97).

### Conclusions

The comparable improvement in QoL at 12 months observed in weekly somatrogon- and daily Genotropin®-treated children aged≥7 years was expected as this clinical trial was powered for non-inferiority (HV at 12 months); hence, the QoL gain should also be comparable between the 2 treatments. Improvement in QoL was demonstrated, regardless of child or caregiver report, by end of Month 12. However, these data show BL and 12-month scores from the QoLISSY-PARENT in both somatrogon and Genotropin® treatment groups were numerically lower than those reported by the child. This is consistent with the literature, in which the caregivers generally report lower QoL scores on behalf of the child. The same pattern was found in other studies with children/adolescents with chronic health conditions, but opposite to that observed in healthy populations. These results may reflect, on the one hand, the children's tendency to emphasize the positive aspects of adaptation and, on the other hand, the parents' reliability in identifying the most strongly affected areas of their children's functioning. Accordingly, routine assessment of pediatric health-related QoL in healthcare and research contexts should include self- and parent-reported data as complementary sources of information.

### Example 4

Perception of Treatment Burden with Once Weekly Somatrogon Vs Once Daily Genotropin® in Pediatric Patients with Growth Hormone Deficiency: Results from a Randomized Phase 3 Study

Objective: Evaluate patient and caregiver perceptions of the treatment burden associated with once weekly somatrogon vs once daily Genotropin®.

Background

Growth hormone deficiency (GHD) is characterized by inadequate secretion of growth hormone from the pituitary gland, and treatment with growth hormone is the standard of care.

Genotropin® is a recombinant human growth hormone (rhGH) with an identical amino acid sequence to the naturally occurring hormone (hGH). It was first approved in the US and other countries in the 1980s and has a well-established safety profile. Genotropin® is administered once daily as a subcutaneous (SC) injection.

In the long term, daily injections may be a burden to the child and his/her caregivers. The introduction of a long-acting rhGH could potentially improve compliance, adherence, and ultimately, clinical outcomes.

Somatrogon is a long-acting rhGH comprised of the amino acid sequence of hGH and 3 copies of the carboxy-terminal peptide from human chorionic gonadotropin. The carboxy-terminal peptides extend the half-life of the attached rhGH, allowing longer intervals between doses. Somatrogon is being developed as a once weekly SC injection for children with GHD. Phase 3 trial results have shown that once weekly somatrogon was generally well tolerated and demonstrated noninferiority to once daily Genotropin® in promoting growth in pediatric GHD.

A recent patient preference study conducted as a discrete-choice experiment showed that patients with GHD preferred a less frequent injection schedule. The current study compared treatment burden of the once weekly somatrogon regimen vs once daily Genotropin® for pediatric GHD.

### Methods

This phase 3, 24-week study (NCT03831880) used a randomized, open-label, crossover design. It was conducted between February 2019 and August 2020, and enrolled patients at centers in Bulgaria, Czech Republic, Slovakia, the UK, and the US.

Eligible patients with pediatric GHD were aged≥3 to <18 years, had insulin-like growth factor-1(IGF-1) standard deviation score<2, and had received stable rhGH therapy for ≥3 months.

Patients were randomized 1:1 to Sequence #1 (12 weeks of once daily Genotropin® followed by 12 weeks of once weekly somatrogon) or Sequence #2 (12 weeks of once weekly somatrogon followed by 12 weeks of once daily Genotropin®). Regardless of the sequence, all patients were to receive a somatrogon dose of 0.66 mg per kg body weight per week, and a Genotropin® dose equivalent to their daily GH dose before the study.

### Assessments

A recently developed, validated Dyad Clinical Outcome Assessment (DCOA) questionnaire was administered electronically. It was to be completed as a Dyad pair (child and caregiver together), with some specific questions intended for the caregiver only.

The DCOA questionnaire is comprised of 2 parts (DCOA 1 and 2), with a comprehensive list of questions to determine the treatment burden. At baseline and after each 12-week treatment period, patients and caregivers completed DCOA 1 (rating treatment experience) and a Patient Global Impression Scale-Impact on Daily Activities (PGIS-IDA).

After experiencing both treatment schedules, they also selected their preference for either daily or weekly injections using questions as part of DCOA 2.

### Endpoints

The primary endpoint was the difference in mean overall life interference total score after each 12-week treatment period, assessed by the Patient Life Interference Questionnaire, a subset of the DCOA 1 questionnaire.

### Results

87 patients were randomized and treated with ≥1 dose of study drug. Patients randomized to the 2 sequences had similar baseline demographics. For the groups receiving Genotropin® first or somatrogon first, respectively, mean (SD) age was 10.8 (3.4) vs 10.7 (3.7) years and 79% vs 86% were male.

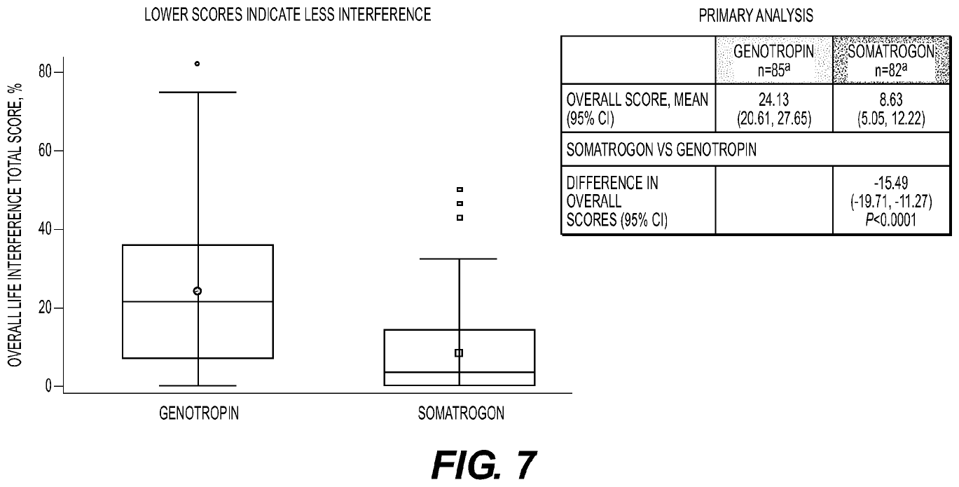

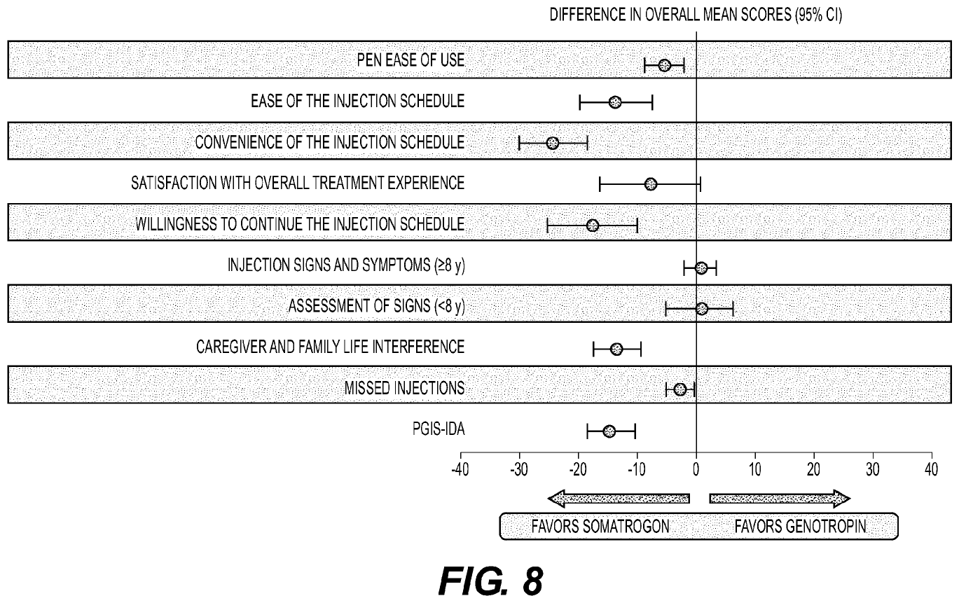

Results of the DCOA 1 questionnaire are summarized in FIG. 7 and FIG. 8. In FIG. 7, all scores were transformed from raw scores and converted to a 0 to 100 scale; model results based on a linear mixed-effects model, including sequence, period, and treatment as fixed effects and subject-within-sequence and within-subject error as random effects; sensitivity analysis in the per protocol set (randomized patients who complete both treatment periods and corresponding assessments; n=81 for both treatments) showed similar results (mean difference in overall scores: −14.85 [95% CI: −19.03, −10.66]; P<0.0001). In FIG. 8, results are based on a linear mixed-effects model, including sequence, period, and treatment as fixed effects and subject-within-sequence and within-subject error as random effects; assessment of signs: caregivers completed the assessment of signs for children<8 years old; all scores were transformed from raw scores and converted to a 0 to 100 scale; lower scores represent less life interference/less impact on daily activities (better outcome); PGIS-IDA=Patient Global Impression Severity-Impact on Daily Activities.

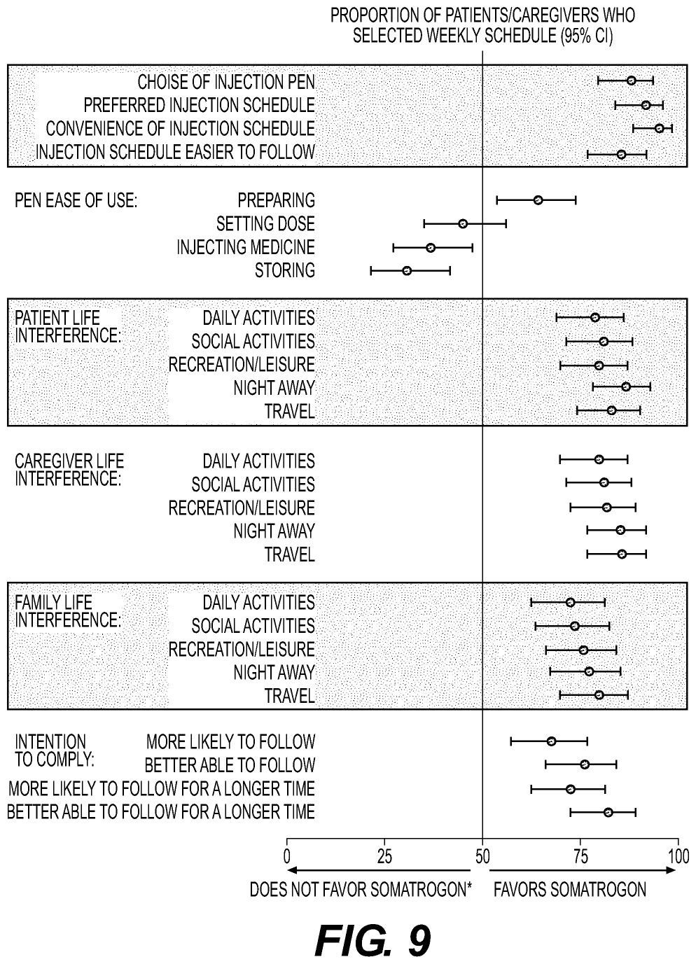

Results of the DCOA 2 questionnaire are summarized in FIG. 9. In FIG. 9, for the 3 items of the “pen ease of use” domain where <50% of patients preferred somatrogon, a substantial proportion of patients had no preference (38.1%, 29.8%, 64.3%, for setting the dose, injecting the medicine, and storing the pen, respectively) between the injection schedules; two-sided 95% CI computed using the Wilson score method.

Once weekly somatrogon demonstrated significant improvement in overall life interference scores vs once daily Genotropin® (FIG. 7). Primary and secondary analyses were conducted in the full analysis set (all randomized patients who received≥1 dose of study drug).

Somatrogon was associated with improvements in other aspects of treatment experience, more patients/caregivers preferred once weekly dosing, and a higher proportion indicated a greater intent to comply with treatment (FIG. 8 and FIG. 9). The caregiver burden was also improved for somatrogon-treated patients.

Safety: Of 87 patients randomized, 86 were treated with both study drugs. During the somatrogon period, 1 patient discontinued the study due to a nonserious treatment-emergent adverse event (TEAE) of moderate injection site pain, considered related to study drug. This patient did not cross over to the Genotropin® period. During the Genotropin® period, 3 patients had temporary discontinuation due to a total of 4 TEAEs (viral upper respiratory tract infection, nasopharyngitis, otitis media and viral infection); all mild and considered not related to study drug.

### Conclusion

In pediatric patients with GHD, compared with once daily Genotropin®, somatrogon administered once weekly has a lower treatment burden as shown by less life interference, and is associated with a more favorable treatment experience.

### Example 5

Phase 3 Study Evaluating Once Weekly Somatrogon Compared to Daily Genotropin® in Japanese Patients with Pediatric Growth Hormone Deficiency (pGHD)

Objective: Assess the efficacy and safety of somatrogon administered once weekly compared with Genotropin® administered once daily in prepubertal Japanese children with GHD.

### Background

Somatrogon is a long-acting recombinant human growth hormone (rhGH) comprising the amino acid sequence of human growth hormone and 3 copies of the carboxy-terminal peptide of human chorionic gonadotropin (SEQ ID NO:2), with a half-life that permits once weekly (QW) administration (Fares et al., Int. J. Cell Biol. (2011) 275063; Fares et al., Proc. Nat. Acad. Sci. USA (1992) 89(10):4304-8). Somatrogon is currently in development as QW treatment for pediatric patients with growth hormone deficiency (GHD).

A phase 3, open-label, randomized study was conducted to compare somatrogon administered QW with Genotropin® administered once daily (QD) in Japanese children with GHD (NCT03874013).

### Methods

This was a 12-month, open-label, randomized, active controlled, parallel-group study. After a 4-week screening period to confirm GHD, subjects were randomized 1:1 to receive either QW somatrogon or QD Genotropin® via subcutaneous injection. QW Somatrogon was administered in 3 escalating doses (0.25, 0.48, and 0.66 mg/kg/wk; 2 weeks at each dose) for 6 weeks, after which subjects continued to receive somatrogon at a dose of 0.66 mg/kg/wk for 46 weeks. QW somatrogon was administered using a single, patient use, multidose, disposable, prefilled pen. QD Genotropin® was administered (0.025 mg/kg/d) using previously approved commercial pen presentations.

During the study, doses of somatrogon and Genotropin® were adjusted every 3 months, based on the subject's body weight. Doses were decreased if required, based on predefined dose-adjustment criteria, which included treatment-related severe adverse events (AEs) and repeated elevated levels of insulin-like growth factor-1 (IGF-1; >+2 standard deviation scores [SDS]). The dose-adjustment criteria and method for this study were the same as described in Example 10 when the IGF-1 level SDS was >+2.

Subjects: Prepubertal boys (ages 3 to <11 y) or girls (ages 3 to <10 y) with a confirmed diagnosis of GHD were eligible for enrolment if they had impaired height (height SDS≤−2), impaired height velocity (HV) below the 25^{th} percentile for chronological age, baseline IGF-1 level that was at least 1 SD below the age- and sex-standard deviation score (SDS≤−1), and had not received prior rhGH therapy. Diagnosis of GHD had to be confirmed by 2 different GH provocation tests (peak serum GH level of ≤6.0 ng/mL or ≤16 ng/mL for a GH-releasing peptide-2 provocation test). Subjects were excluded if they had any prior history of cancer or had received radiation therapy or chemotherapy. Subjects who were malnourished (body mass index has a SDS of <−2 [age and sex standardized]), were born small for their gestational age, or had anti-hGH antibodies at screening, diabetes mellitus, psychosocial dwarfism, or known or suspected chromosomal abnormalities or genetic/epigenetic variants (including Turner syndrome, Laron syndrome, Noonan syndrome, Prader-Willi syndrome, Silver-Russell syndrome, SHOX mutations/deletions, and skeletal dysplasias) were also excluded from the study.

Assessments and Endpoints: Height measurements were performed at screening, baseline, and Weeks 13, 26, 39, and 52 (end of treatment) using a calibrated stadiometer; 3 independent readings were recorded for each visit and the mean was calculated. Height SDS was derived from age and gender according to the local primary care provider standard (national survey data from 2000) (Ministry of Health, Labour and Welfare of Japan, world wide web: ispe.umin.jp/medical/taikaku.html). Annualized HV was calculated as the change in height from visit 2 (baseline) to visit 9 (month 12). Bone age was determined via X-ray according to the Tanner-Whitehouse 2 protocol using a central bone age reader at screening or baseline, and week 52.

Adverse events (AEs), including injection site reactions, were assessed at each study visit, with the exception of injection site reactions, which were not assessed at predose visits; subjects were also trained to record injection site reactions in a diary.

PK and PD assessments: For subjects in the somatrogon group, blood samples were collected 12 to 120 hours post dose for analysis of serum somatrogon and IGF-1 in accordance with sampling sub-blocks. For each of the 3 dose groups, 2 samples were collected at different times after administration of the second dose, with a total of 6 samples collected for each subject. Median concentrations were calculated for each dose using naïve pooled estimate at each time point. Subjects who received Genotropin had blood samples collected regularly over the study period. Assessment of anti-drug antibodies (ADAs) against somatrogon and Genotropin were performed at protocol-specified time points by Eurofins Pharma Bioanalytics Services US Inc. The development of binding and/or neutralizing antibodies against somatrogon was assessed using qualitative, validated methods (Zelinska et al., J. Clin. Endocrinol. Metab. (2017) 102(5):1578-87).

Safety: Safety evaluations included all adverse events (AEs), concomitant medication use, treatment compliance, vital signs, electrocardiogram, physical examination, and laboratory assessments (hematology, blood chemistry, glucose metabolism, endocrinology, IGF-1 levels, immunogenicity, and urinalysis). An AE was defined as any adverse change from the subject's condition at baseline, regardless of whether it was considered related to the investigational product. AEs (including injection-site reactions) were assessed at all study visits; however, injection site reactions were not assessed at pre-dose visits 7 (month 6) and 9 (month 12). The intensity or severity of an AE was characterized as mild, moderate, or severe. Subjects recorded data on AEs, concomitant medications, and injection site reactions at home using a patient diary.

Any injection-site reactions that met the criteria for “abnormal” were assessed as AEs. An abnormal injection-site reaction was defined as a reaction that was moderate to severe in intensity, required medical attention, was deemed abnormal by an investigator, or had a pain score≥4, based on the protocol-specified Pain Assessment Scale (ranging from 0 [“no hurt”] to 5 [“hurts worse”]). In the somatrogon group, the severity of injection-site pain after each weekly injection was recorded. In contrast, in the Genotropin® group, only the most severe pain for the week was recorded (i.e., once a week), rather than after each daily injection. Where a Genotropin® subject experienced multiple events of pain score≥4, only 1 occurrence was recorded in the diary, hence only 1 AE would be recorded.

Statistical Analyses: Comparability of once-weekly somatrogon and once-daily Genotropin was concluded for the primary efficacy endpoint if the point estimate of the mean treatment difference (somatrogon-Genotropin) was ≥−1.8 cm/year. The pre-established mean treatment difference of −1.8 cm/year was the noninferiority margin used in the Phase 3 global study comparing once-weekly somatrogon with once-daily Genotropin. The mean and 95% confidence interval (CI) for HV at 12 months and the point estimate of the treatment difference were calculated using the least square (LS) means from an analysis of covariance (ANCOVA) model. The secondary efficacy endpoints were annualized HV following 6 months of treatment, change in height SDS at 6 and 12 months (compared with pretreatment), and change in bone maturation (defined as the ratio of bone age to chronological age) after 12 months (compared with bone maturation pretreatment).

TABLE 6
Subject demographics and baseline characteristics (safety analysis set).

| | Somatrogon | | Genotropin | | Total | |
|---|---|---|---|---|---|---|
| | (n = 22) | | (n = 22) | | (N = 44) | |
| Mean (SD) age, years | 5.28 | (1.84) | 6.78 | (2.34) | 6.03 | (2.21) |
| Age group, n (%) | | | | | | |
| ≥3 to ≤7 years | 19 | (86.4) | 12 | (54.5) | 31 | (70.5) |
| >7 years | 3 | (13.6) | 10 | (45.5) | 13 | (29.5) |
| Sex, n (%) | | | | | | |
| Male | 9 | (40.9) | 12 | (54.5) | 21 | (47.7) |
| Female | 13 | (59.1) | 10 | (45.5) | 23 | (52.3) |
| Peak GH level group,^{a} n (%) | | | | | | |
| Low | 1 | (4.5) | 1 | (4.5) | 2 | (4.5) |
| High | 21 | (95.5) | 21 | (95.5) | 42 | (95.5) |
| IGF-1 SDS (Z-score) | | | | | | |
| Mean (SD) | −1.39 | (0.90) | −1.62 | (0.84) | −1.50 | (0.87) |
| Median | −1.46 | | −1.42 | | −1.42 | |
| Range (min, max) | −3.48, 0.64 | | −3.74, −0.52 | | −3.74, 0.64 | |
| Mean IGF-1 (SD), μg/L | 72.9 | (33.5) | 80.5 | (30.7) | 76.7 | (32.0) |
| Height SDS (Z-score) | | | | | | |
| Mean (SD) | −2.61 | (0.44) | −2.53 | (0.40) | −2.57 | (0.42) |

| | | | |
|---|---|---|---|
| Median | −2.70 | −2.48 | −2.59 |
| Range (min, max) | −3.38, −1.83 | −3.45, −1.83 | −3.45, −1.83 |

Abbreviations: GH, growth hormone; GHRP-2, growth hormone-releasing peptide 2; IGF, insulin-like growth factor; max, maximum; min, minimum; SDS, standard deviation score.
^{a}Based on: ≤3 ng/mL or >3 to ≤6 ng/mL; or if GHRP-2 provocation test is used, ≤10 ng/mL or >10 to ≤16 ng/mL.

### Results

Study Participants: 65 subjects were screened and 44 subjects randomized at 24 sites in Japan; of the 44 dosed subjects, 43 completed the 12-month main study, and 1 subject in the Genotropin® group discontinued from the study due to an AE (craniopharyngioma). Demographic and baseline characteristics were similar between the 2 treatment groups (somatrogon and Genotropin®), with most (70%) subjects aged between 3 and 7 years. Approximately half (47.7%) of the subjects were male.

Efficacy: The least squares (LS) mean of height velocity (HV) at Month 12 was 9.65 cm/y in the somatrogon group and 7.87 cm/y in the Genotropin® group; similar results were observed for annualized HV at Month 6. LS mean treatment difference of +1.79 cm/y (95% CI: 0.97-2.60) in HV at Month 12 was greater than the pre-established margin of −1.8 cm/y, demonstrating QW somatrogon was comparable to QD Genotropin®.

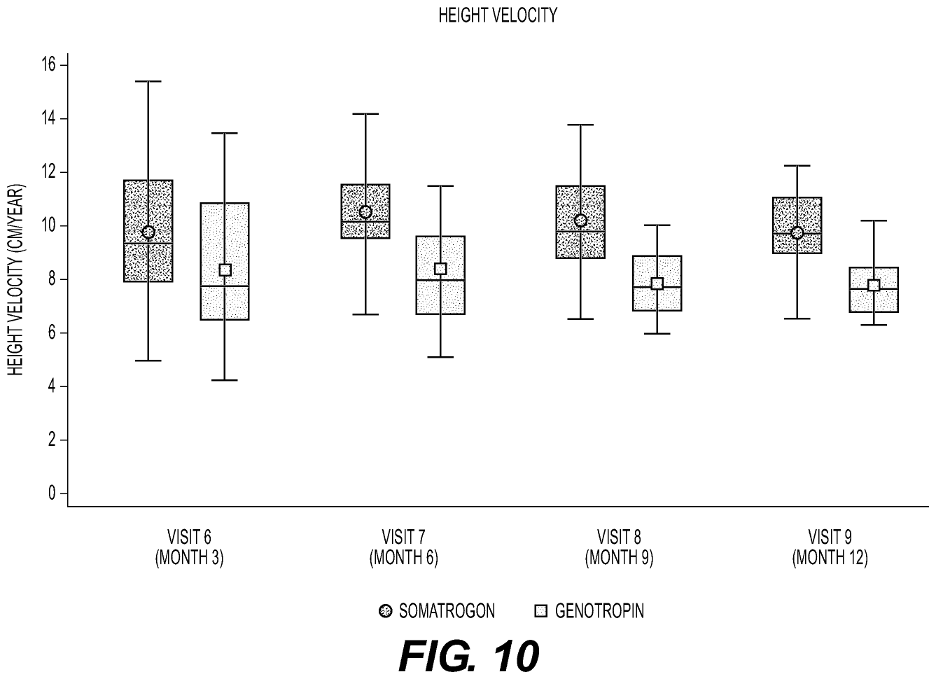

The LS mean for HV at month 6 in the somatrogon group (10.35 cm/year) was also higher than in the Genotropin group (8.47 cm/year). The resulting treatment difference for the LS mean HV was +1.88 cm/year (95% CI, 0.74-3.03). Mean HV values for the somatrogon group were higher than for the Genotropin group at months 3, 6, 9, and 12 of the study (FIG. 10).

At 6 and 12 months, respectively, mean height SDS was higher in the somatrogon group (−2.02 and −1.64) compared with the Genotropin® group (−2.23 and −2.03). The LS mean change from baseline in height SDS at 6 and 12 months, respectively, was higher in the somatrogon group (0.58 and 0.94) compared with the Genotropin® group (0.31 and 0.52).

The mean treatment difference for change in height SDS at 12 months was +0.42 (95% CI, 0.23-0.61). A similar trend was observed for the change in height SDS from baseline to 6 months, with a mean treatment difference of +0.26 (95% CI, 0.12-0.41).

Advancement in bone age (BA) did not exceed advancement in chronological age (CA). The mean (SD) change in bone maturation at 12 months was 0.052 (0.065) and 0.035 (0.062) for the somatrogon and Genotropin® groups, respectively. Mean bone maturation (defined as the ratio of BA to CA) at 12 months was <1.0 in both treatment groups (somatrogon: 0.80; Genotropin®: 0.80).

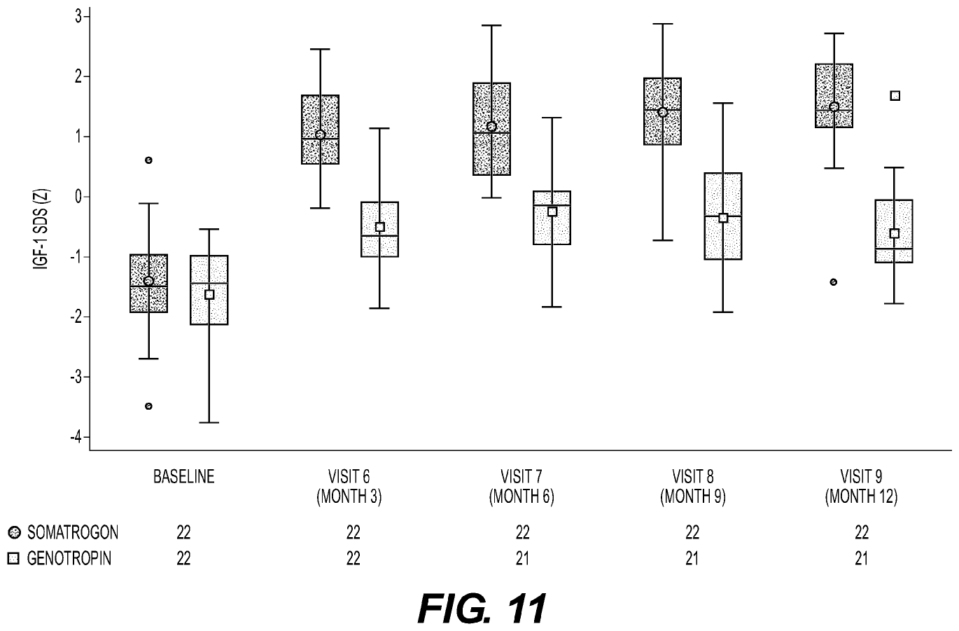

The mean IGF-1 SDS (relative to baseline) increased across all post-baseline visits for the somatrogon treatment group (FIG. 11). In the Genotropin® treatment group, mean IGF-1 SDS increased until month 6 and decreased at months 9 and 12. From week 2, mean IGF-1 SDS values in the somatrogon group approached 0 SDS and remained above 0 SDS through month 12. Mean IGF-1 SDS values in the Genotropin® group ranged from −0.59 to −0.25 SDS at all post-baseline visits.

PK/PD: Peak serum concentrations of somatrogon were achieved at 12 hours to 18 hours after dosing, and the maximum concentration of somatrogon increased with increasing dosage. The calculated median concentrations of IGF-1, IGF-1 SDS, and IGFBP-3 based on sparse sampling showed that somatrogon treatment resulted in an IGF-1 and IGFBP-3 response. Median IGF-1 standard deviation scores (SDS) did not exceed +2 through the course of the week or at regular study visits over 12 months (FIG. 11).

Safety: The mean duration of treatment was 367.6 days in the somatrogon group and 344.6 days in the Genotropin® group. A total of 22/22 (100.0%) and 19/22 (86.4%) subjects reported all-causality AEs in the somatrogon and Genotropin® groups, respectively (Table 7). The number of subjects with all-causality treatment-emergent AEs (TEAEs) were similar between treatment groups. Subjects in the somatrogon group had a higher incidence of TEAEs vs the Genotropin® group (359 vs. 106 events); TEAE of injection site pain was the primary cause for the difference in the incidence of TEAEs between groups (205 vs 8 events).

The most common all-causality TEAEs were nasopharyngitis (somatrogon: 54.5%; Genotropin®: 50.0%), injection site pain (somatrogon: 72.7%; Genotropin®: 13.6%), influenza (somatrogon: 27.3%; Genotropin®: 27.3%), pyrexia (somatrogon: 18.2%; Genotropin®: 13.6%), and pharyngitis (somatrogon: 13.6%; Genotropin®: 18.2%); the majority of TEAEs were mild to moderate in severity (somatrogon: 90.9%; Genotropin®: 77.3%).

The most common treatment-related TEAE was injection site pain (pain score≥4): somatrogon: 16/22 (72.7%), Genotropin®: 3/22 (13.6%). Although the somatrogon group had a higher proportion of subjects with injection site pain scores≥4, the proportion of subjects who reported any injection site pain (pain scores: 1-5) was similar between the somatrogon (100%) and Genotropin® groups (91%). The proportion of subjects reporting lower pain scores (1-3) was higher in the Genotropin® group (77.3%) compared with the somatrogon group (27.3%). The severity of all reported AEs of injection-site pain in the somatrogon group was mild (15 subjects), with the exception of 1 subject who reported moderate injection-site pain. None of the subjects reported severe injection-site pain. Most events of injection-site pain were reported during the first 6 months of the study.

The incidence of serious AEs was low in both treatment groups; treatment-emergent serious AEs were reported by 2 (9.1%) subjects in the somatrogon group (hypoparathyroidism, influenza, traumatic fracture, and febrile convulsion) and 2 (9.1%) subjects in the Genotropin® group (craniopharyngioma and asthma) (Table 7). The event of craniopharyngioma reported in the Genotropin group was classified by the investigator as a treatment-related SAE and resulted in the subject being discontinued from the study.

No deaths occurred during the study, and no subjects had a dose reduction due to an AE. For the majority of subjects in both treatment groups, blood glucose and HbA1c (%) levels remained in the normal range for the following post-baseline visits: months 1, 3, 6, 9, and 12 for blood glucose and months 6 and 12 for HbA1c. Similarly, levels of thyrotropin and free thyroxine remained within the normal range for most post-baseline visits (months 3, 6, 9, and 12) for the majority of subjects in both treatment groups. Overall, there were no clinically meaningful differences observed between treatment groups in terms of glucose metabolism, hematology, chemistry, thyroid function, lipid profiles, and urinalysis parameters. The number of subjects in the somatrogon group who reported IGF-1 SDS>+2 at each visit was as follows: month 3: 3 (13.6%) subjects; month 6: 4 (18.2%) subjects; month 9: 5 (22.7%) subjects; and month 12: 6 (27.3%) subjects. None of the subjects in the Genotropin® group reported IGF-1 SDS>+2.

TABLE 7
Treatment-emergent adverse events (all causalities) - safety analysis set

| | Somatrogon | | Genotropin | | Total | |
|---|---|---|---|---|---|---|
| | n (%) | | n (%) | | N (%) | |
| Subjects evaluable for AEs | 22 | | 22 | | 44 | |
| Number of AEs | 359 | | 106 | | 465 | |
| Subjects with AEs | 22 | (100.0) | 19 | (86.4) | 41 | (93.2) |
| Subjects with SAEs | 2 | (9.1) | 2 | (9.1) | 4 | (9.1) |
| Subjects with severe AEs | 2 | (9.1) | 2 | (9.1) | 4 | (9.1) |

| | | | | | |
|---|---|---|---|---|---|
| Subjects discontinued from study due to AE^{a} | 0 | 1 | (4.5) | 1 | (2.3) |

| | | | |
|---|---|---|---|
| Subjects discontinued study drug due to AE | 0 | 0 | 0 |
| and continue study^{b} | | | |
| Subjects with dose reduced or temporary | 0 | 0 | 0 |
| discontinuation due to AEs | | | |

Abbreviations: AE, adverse event; SAE, serious adverse event. SAEs are based on the investigator's assessment.
^{a}Subjects who have an AE record that indicates that the AE caused the subject to be discontinued from the study.
^{b}Subjects who have an AE record that indicates that action taken with study treatment was drug withdrawn but AE did not cause the subject to be discontinued from study.

Immunogenicity: A total of 18/22 subjects in the somatrogon group and 4/22 subjects in the Genotropin® group tested positive for ADAs during the 12-month treatment period. Most subjects in the somatrogon group who tested positive for ADAs had ADAs that were specific for the hGH component of somatrogon. Two subjects in the somatrogon group tested positive for neutralizing antibodies (nAbs) against somatrogon at a single visit but were negative for all subsequent visits. None of the subjects in the Genotropin® group tested positive for nAbs.

There were no differences between somatrogon recipients who were ADA-positive compared with those who were ADA-negative, suggesting that the presence of ADAs to somatrogon did not have an effect on the efficacy or safety of the treatment during the 12-month study.

### Conclusions

The study met its primary objective: somatrogon administered once weekly was comparable to Genotropin® administered once daily with regard to annual HV after 12 months of treatment. The mean HV and height SDS were numerically higher in the somatrogon group across all post-baseline visits in comparison with the Genotropin® group.

Somatrogon administered once weekly was concluded as being comparable to Genotropin® administered once daily as the mean treatment difference (somatrogon-Genotropin®) in HV was +1.79 cm/year (95% CI, 0.97-2.60), which was greater than the pre-established margin of −1.8 cm/year. Compared with the Genotropin® group, the somatrogon group had higher HV at 12 months (9.65 cm/year vs 7.87 cm/year) and showed a greater improvement in height SDS from baseline to 12 months (0.94 vs 0.52). Both treatment groups showed similar changes in bone maturation; advancement in bone age did not exceed advancement in chronological age.

The pain and discomfort of daily GH injections have been identified by patients and caregivers as some of the key burdens associated with daily GH treatment (Brod et al., Patient (2017) 10(5):653-66; Graham et al., Patient Prefer. Adherence (2020) 14:1889-99; Kremidas et al., J. Pediatr. Nurs. (2013) 28(1):55-63). Other treatment burdens identified included a fear of injections, the requirement to store/reconstitute medication, and life interference associated with daily injections. These burdens are likely to influence adherence to treatment, which is critical for treatment efficacy. A recent systematic review reported that nonadherence to rhGH treatments may be as high as 71% (Horm. Res. Paediatr. (2018) 90(4):221-27). Nonadherence may reduce the efficacy of GH treatment, resulting in suboptimal growth responses and reduced HV and final adult height (Graham et al., Patient Prefer. Adherence (2020) 14:1889-99). Reducing the number of rhGH injections required is likely to significantly lower the treatment burden associated with GH treatment, which may encourage greater adherence in patients and caregivers. As such, somatrogon administered once weekly may alleviate many of the issues associated with compliance with once-daily Genotropin. Although the somatrogon group had a higher incidence of injection-site pain compared with the Genotropin group, subjects may prefer to receive 1 injection of somatrogon compared with 7 injections of Genotropin during the course of a week. This is supported by a discrete choice experiment conducted in Japanese children with GHD, which found a clear preference for a once-weekly injection schedule instead of a once-daily injection schedule (Tanaka et al., Pediatr. Int. (2021)). Although the use of once-weekly somatrogon may improve adherence among patients with GHD, it is possible that some patients with poor adherence to daily GH treatment (e.g., adolescents) will also show poor adherence to long-acting treatments such as once-weekly somatrogon.

IGF-1 concentrations may be monitored to assess compliance and response to treatment. With daily rhGH, there is no concern about the time after dose for collection of samples because the peak:trough ratios are small and any observations are reflective of what would be observed over the dosing interval. With once-weekly treatment, as was shown in this study, the peak:trough variability is larger. A PK/PD analyses performed on the Phase 2 study and showed that mean IGF-1 SDS over the 1-week dosing interval was best approximated by IGF-1 assessments 4 days (96 hours) after dose administration (Fisher et al., Horm. Res. Paediatr. (2017) 87(5):324-32).

Somatrogon administered once weekly was generally well-tolerated in children with GHD. The results of this Japanese phase 3 study are consistent with those reported from the global phase 3 study that met its primary endpoint of non-inferiority to Genotropin® administered once daily.

### Example 6

Patient Counseling for Treatment with Once Weekly Long-Acting rhGH, Contraindications, Warnings and Precautions, Drug Interactions, and Use in Specific Populations

This Example illustrates patient counseling information to be provided to the patient and/or caregiver when administering long-acting rhGH, contraindications, warnings and precautions, drug interactions, and use in specific populations.

### Patient Counseling

Advise the patient and/or caregiver to read the FDA-approved patient labeling (Patient Information and Instructions for Use).

Adrenal Cortical Hypofunction: hypoadrenalism may develop in patients who have or who are at risk for pituitary hormone deficiency(s). Advise patients/caregivers that if hyperpigmentation, extreme fatigue, dizziness, weakness, or weight loss is experienced during treatment with somatrogon to report this to their healthcare provider.

Thyroid Function: undiagnosed/untreated hypothyroidism may prevent an optimal response to the long-acting rhGH. Advise patients/caregivers that periodic thyroid function tests may be required during treatment with the long-acting rhGH.

Benign Intracranial Hypertension: advise patients/caregivers to report any visual changes, headache, and nausea and/or vomiting to their healthcare provider.

Hypersensitivity Reaction: advise patients/caregivers that serious systemic hypersensitivity reactions (anaphylaxis and angioedema) are possible and that prompt medical attention should be sought if an allergic reaction occurs.

Glucose Metabolism: advise patients/caregivers that new onset of insulin resistance and hyperglycemia may occur and monitoring of blood glucose during treatment with the long-acting rhGH in patients with glucose intolerance or who have risk factors for diabetes, may be needed.

Never Share a long-acting rhGH Pen Between Patients. Advise patients/caregivers that the long-acting rhGH pen should not be shared with another person even if the needle is changed, because doing so carries a risk for transmission of blood-borne pathogens.

Patients and caregivers who will administer the long-acting rhGH should receive appropriate training and instruction on the proper use and handling of the long-acting rhGH from the physician or other suitably qualified health care professional.

### Contraindications

Active Malignancy: Based on experience with daily growth hormone products, long-acting rhGH is contraindicated in patients with active malignancy.

Acute Critical Illness: Based on experience with pharmacologic amounts of daily growth hormone products, long-acting rhGH is contraindicated in patients with acute critical illness due to complications following open heart or abdominal surgery, multiple accidental trauma, or acute respiratory failure.

Hypersensitivity: The long-acting rhGH is contraindicated in patients with known hypersensitivity to the long-acting rhGH or any of its excipients.

### Warnings and Precautions

Acute Critical Illness: Treatment with pharmacologic amounts of daily growth hormone products has been associated with increased mortality in patients with acute critical illness due to complications following open heart surgery, abdominal surgery or multiple accidental trauma, or those with acute respiratory failure. Based on experience with daily growth hormone products, if patients who are receiving the long-acting rhGH therapy become acutely critically ill, the potential benefit of continued treatment should be weighed against the potential risk.

Glucose Metabolism: Treatment with daily growth hormone products may induce a state of insulin resistance and hyperglycemia. Additional monitoring should be considered in patients treated with the long-acting rhGH who have glucose intolerance, or additional risk factors for diabetes. In patients treated with the long-acting rhGH who have diabetes mellitus, anti-diabetic therapy may require adjustment.

Benign Intracranial Hypertension: Intracranial hypertension (IH) with papilledema, visual changes, headache, nausea, and/or vomiting has been reported in a small number of patients treated with daily growth hormone products. Symptoms usually occurred within the first 8 weeks after the initiation of daily growth hormone therapy. In all reported cases, IH-associated signs and symptoms rapidly resolved after cessation of therapy or a reduction of the daily growth hormone dose. The long-acting rhGH should be temporarily discontinued in patients with clinical or fundoscopic evidence of IH.

Hypersensitivity Reactions: Serious systemic hypersensitivity reactions (e.g., anaphylaxis, angioedema) have been reported with daily growth hormone products. If a serious hypersensitivity reaction occurs, immediately discontinue use of the long-acting rhGH therapy; treat promptly per standard of care and monitor until signs and symptoms resolve. Do not use in patients with previous hypersensitivity to the long-acting rhGH therapy.

Adrenal Cortical Hypofunction: Based on published data patients receiving daily growth hormone therapy who have or are at risk for pituitary hormone deficiency(s) may be at risk for reduced serum cortisol levels and/or unmasking of central (secondary) hypoadrenalism. In addition, patients treated with glucocorticoid replacement for previously diagnosed hypoadrenalism may require an increase in their maintenance or stress doses following initiation of the long-acting rhGH therapy treatment. Monitor patients for reduced serum cortisol levels and/or need for glucocorticoid dose increases in those with known hypoadrenalism.

Thyroid Function: Based on experience with daily growth hormone products, undiagnosed/untreated hypothyroidism may prevent an optimal response to the long-acting rhGH therapy. During the long-acting rhGH therapy, thyroid function should be monitored as indicated based on clinical evaluation.

Epiphyseal Disorders: Epiphyseal disorders, including slipped capital femoral epiphysis, may occur more frequently in patients with endocrine disorders or in patients undergoing rapid growth. Any pediatric patient with the onset of a limp or complaints of hip or knee pain during treatment should be carefully evaluated.

### Drug Interactions

Glucocorticoids: In patients receiving concomitant the long-acting rhGH therapy and glucocorticoid treatments, glucocorticoid dosing should be carefully monitored to avoid both hypoadrenalism and an inhibitory effect on growth. The microsomal enzyme 11β-hydroxysteroid dehydrogenase type 1 (11βHSD-1) is required for conversion of cortisone to its active metabolite, cortisol, in hepatic and adipose tissue. Treatment with daily growth hormone products inhibits 11β1-1SD-1, reducing serum cortisol concentrations, which may unmask previously undiagnosed central (secondary) hypoadrenalism or render low glucocorticoid replacement doses ineffective. Patients treated with cortisone acetate and prednisone may be affected more than others because conversion of these drugs to their biologically active metabolites is dependent on the activity of 11βHSD-1.

Insulin and/or Oral/Injectable Hypoglycemic Agents: In patients with diabetes mellitus requiring drug therapy, the dose of insulin and/or oral/injectable agent may require adjustment when the long-acting rhGH therapy is initiated.

### Use in Specific Populations

Pregnancy Risk Summary: To minimize risk of major birth defects and miscarriage, the long-acting rhGH therapy should be used during pregnancy only if clearly needed.

Lactation: The long-acting rhGH therapy should be administered to lactating women only if clearly needed.

Females and Males of Reproductive Potential Pregnancy: Somatrogon has been shown not to interfere with blood or urine pregnancy tests.

Pediatric Use: The safety and effectiveness of the long-acting rhGH therapy has been evaluated in pediatric patients aged 3 years and older with growth failure due to GHD.

Geriatric Use: The safety and effectiveness of the long-acting rhGH therapy in adult patients have not been established.

### Example 7

### Pharmacodynamics and Pharmacokinetics

This Example illustrates pharmacodynamics and pharmacokinetics of somatrogon.

Somatrogon increases IGF-1. Pharmacodynamic evaluations were performed approximately 96 hours after dose administration in order to assess the mean IGF-1 SDS over the dosing interval.

Somatrogon pharmacokinetics (PK) was assessed using a population PK approach for somatrogon in 42 pediatric patients with GHD. Following SC injection, serum concentrations increased slowly, peaking 6 to 18 hours after dosing. In pediatric patients with GHD, somatrogon exposure increases in a dose-proportional manner for doses of 0.25 mg/kg/wk, 0.48 mg/kg/wk, and 0.66 mg/kg/wk. There is no accumulation of somatrogon after once weekly administration. In pediatric patients with GHD, the mean population PK estimated steady-state peak concentrations following 0.66 mg/kg/wk was 690 ng/mL. In pediatric patients with GHD, the mean population PK estimated apparent central volume of distribution was 0.812 L/kg and apparent peripheral volume of distribution was 0.169 L/kg. In pediatric patients with GHD, the mean population PK estimated apparent clearance was 0.0336 L/h/kg. With a mean population PK estimated effective half-life of 28.3 hours, somatrogon will be present in the circulation for about 6 days after the last dose. Based on population PK analyses, age, sex, race, and ethnicity do not have a clinically meaningful effect on the pharmacokinetics of somatrogon in pediatric patients with GHD. The exposure of somatrogon decreases with an increase in body weight. However, the somatrogon dosing regimen of 0.66 mg/kg/wk provide adequate systemic exposure over the body weight range of 10 to 54 kg evaluated in the clinical studies.

### Example 8

### Glycosylation Pattern of a Recombinant Long-Acting Growth Hormone

Somatrogon is a glycoprotein produced in Chinese Hamster Ovary (CHO) cells by recombinant DNA technology. It is comprised of the amino acid sequence of human growth hormone (hGH) with one copy of the C-terminal peptide (CTP) from the beta chain of human chorionic gonadotropin (hCG) at the N-terminus and two copies of CTP (in tandem) at the C-terminus. Each CTP includes multiple O-linked glycosylation sites. The glycosylation and CTP domains prolong the half-life of somatrogon, which allows for weekly dosing. The O-glycan occupancy ranges from 9 to 20 moieties per intact somatrogon molecule. The predominant somatrogon glycoforms include the molecule with 15 monosialylated, core-1 O-glycans or 16 monosialylated, core-1 O-glycans. Additionally, each CTP region contains hydroxyproline residues, which range from 0-5 hydroxy additions per intact somatrogon molecule.

The amino acid sequence of somatrogon is shown in SEQ ID NO: 2. The functional, intact molecule is composed of recombinant hGH and one copy of CTP from the beta chain of hCG at the N-terminus (amino acids residues 1-28) and two copies of CTP (in tandem) at the C-terminus (amino acids residues 220-247 and 248-275.

The theoretical molecular masses (average) of the aglycosylated and predominant O-linked glycoforms, with full disulfide bond connectivity, are provided in Table 8. ESI MS was used to confirm the primary structure and posttranslational modifications of intact somatrogon, as well as identify the major and minor product isoforms.

TABLE 8
Theoretical molecular mass and Formulas for somatrogon

| O-linked Glycoform | Theoretical Mass (Da) | Molecular Formula |
|---|---|---|
| Aglycosylated | 30465.1 | C₁₃₅₉H₂₁₂₅N₃₆₁O₄₂₀S₇ |
| 15 O-glycans, core-1 | 40313.9 | C₁₇₃₄H₂₇₂₅N₃₉₁O₆₉₀S₇ |
| monosialylated | | |
| (GalNAc-Gal-NeuAc) | | |
| 16 O-glycans, core-1 | 40970.5 | C₁₇₅₉H₂₇₆₅N₃₉₃O₇₀₈S₇ |
| monosialylated | | |
| (GalNAc-Gal-NeuAc) | | |

The predominant glycoform had experimental molecular mass of 40314.4 Da, which is consistent with the correct amino acid sequence for somatrogon with 15 core-1 monosialylated O-glycans and two disulfide bonds (theoretical molecular mass=40313.9 Da). The experimental masses of two additional predominant 0-glycoforms, 39657.3 and 40970.3 Da agree well with theoretical values of somatrogon glycoforms consisting of the intended amino acid sequence with two disulfide bonds and 14 or 16 core-1 monosialylated O-glycans, respectively. Compositionally, these respective accurate masses indicate that somatrogon contains a single polypeptide chain with the correct amino acid sequence, two disulfide bonds, as well as the expected core-1 monosialylated O-glycans. Additional minor and trace level isoforms were detected by ESI MS, which is consistent with the expected O-linked oligosaccharide heterogeneity. The core-1 monosialylated O-glycans observed in ESI MS of somatrogon RM range from 10-18 for the intact protein. Up to 19 O-glycans have been previously observed. Other minor and trace-level 0-glycoforms were composed of asialylated and di-sialylated core-1 O-glycans. Additionally, each CTP region contains 0-5 hydroxy additions per intact somatrogon molecule.

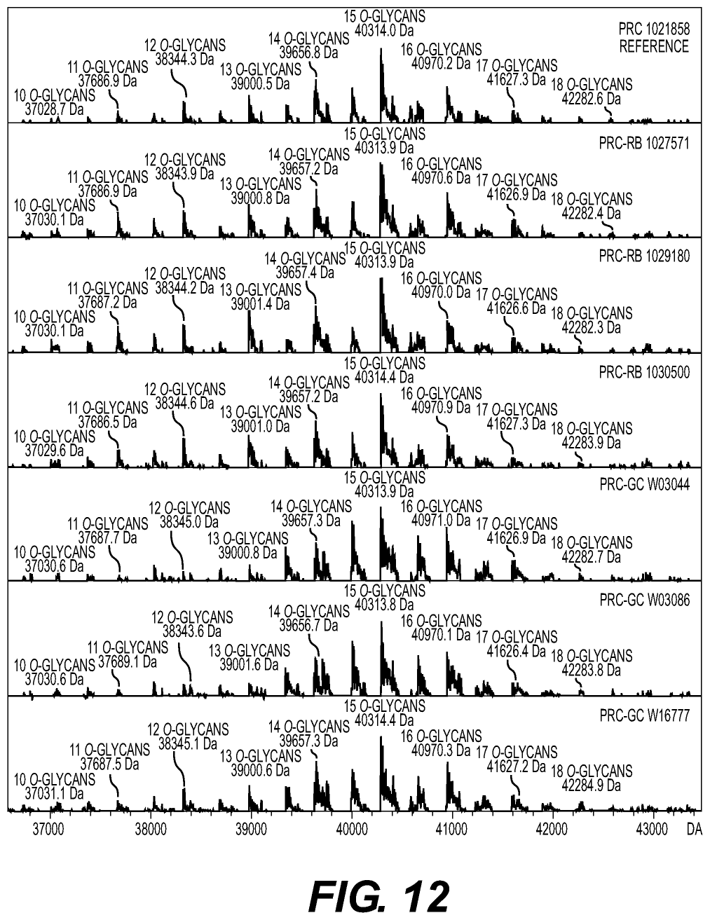

Accurate relative molecular masses (Mr) of the intact somatrogon glycoforms of PRC-RB and PRC-GC drug substance were determined by size-exclusion chromatography with online electrospray ionization mass spectrometry detection (ESI MS). The mass spectra for the intact somatrogon materials is shown in FIG. 12. In FIG. 12, The major distribution is labeled with the number of O-glycans observed based on the relative molecular mass. The predominant glycoform had Mr consistent with the correct amino acid sequence with 15 monosialylated core-1 O-glycans and 2 disulfide bonds (theoretical Mr=40313.9 Da). The major O-glycosylation distribution for each material ranged from 10 to 18 O-glycans. An additional, minor glycoform distribution was observed in all samples and corresponded to the major distribution species±sialic acid (N-acetylneuraminic acid, NeuAc). In FIG. 12, the minor unlabelled distribution represents major isoforms±sialic acid. Hydroxyproline was also detected for each O-glycoform in all somatrogon materials in similar proportions. When the mass spectra of intact PRC-GC and PRC-RB are compared, no new glycoforms are observed in PRC-GC as compared to PRC-RB. Only a minor redistribution in the relative abundance of 0-glycoforms is observed. The intact somatrogon mass spectrometry assessment confirms that the PRC-GC and PRC-RB materials are comparable at the intact level.

### Example 9

Pediatric Growth Hormone Disease Patients' Adherence with and Discontinuation of Daily Growth Hormone in a U.S. Commercial Claims Database

Objectives: To describe the adherence and discontinuation of somatropin among pediatric patients with growth hormone deficiency (GHD) treated with somatropin over 4 years. To evaluate important demographic characteristics associated with time to discontinuation of GHD treatment.

One in every 4,000 children in the US suffers from the pediatric growth hormone deficiency (pGHD). Patients with pGHD are presented with short stature, and they are managed with daily injections of somatropin, a daily growth hormone (dGH). It has been reported that the adherence to somatropin has been suboptimal. Compared to those with suboptimal adherence to dGH injection, adherent children have demonstrated significantly greater linear growth. To date, suboptimal adherence, and discontinuation with dGH injections has not been studied among large, usual-care populations using validated measurements of adherence.

Study Design: A retrospective database analysis of pediatric GHD patients (aged 3-15 years) who were newly treated with somatropin was conducted using Optum Clinformatics Data Mart database. Index date was defined as the first prescription for somatropin between 1 Jul. 2002 to 30 Sep. 2019. Patients were followed for up to 48 months from the time of the first somatropin prescription, and 5 non-exclusive cohorts were constructed (3, 12, 24, 36, 48 months of post-index).

Key inclusion criteria: Patients aged 3-15 years with the continuous enrollment. >1 diagnosis code for pGHD during 6 months prior to or on the index date≥2 prescription claims for somatropin between Jul. 1, 2002 and Sep. 30, 2019. First somatropin claim during this window denotes the index date. Key exclusion criteria: Claims for somatropin during 6 months prior to or on the index date. Other causes of short stature such as psychosocial dwarfism, celiac disease, uncontrolled primary hypothyroidism and rickets.

The demographic and clinical profiles of children with pGHD treated with daily injections of somatropin were characterized.

Descriptive analyses were performed for all study variables. Adherence with somatropin was defined using medication possession ratio. Patients classified as having good (>80%) or suboptimal (<80%) adherence. Discontinuation was defined as the first observation of a gap of >60 days between somatropin prescription fills among those followed for >3 months. Cox proportional hazards models were fitted to analyze time to discontinuation (TTD).

### Results

A total of 21,260 individuals had 2 or more somatropin prescriptions between Jul. 1, 2002 and Sep. 30, 2019. Discontinuation was evaluated in 3,969 who had >3 months of available follow-up. Patient characteristics were similar across each cohort; demographic characteristics of patients 3-month, 12-month, 48-month cohorts.

Overall, among the 12, 24, 36, and 48 month follow-up cohorts, 19.6%, 29.8%, 34.2%, and 35.9% of pGHD patients had suboptimal somatropin adherence. Suboptimal adherence was most pronounced among black and Hispanic children. At 48 months, suboptimal adherence was observed among 44.8% of Hispanics, 43.2% of blacks, 34.6% of whites, and 26.1% of Asians. Sensitivity analysis was performed, and the pattern remained the same for the patients followed for 48 months as compared to the primary analysis.

42.2% of patients discontinued somatropin therapy among all pGHD patients with at least 3 months of follow-up. Half of patients who discontinued somatropin did so within 1.2 years. Among those who discontinued therapy, mean time to discontinuation was 524.4 days (SD 374.6, median 442.0). In adjusted models (Table 9) the factors associated with increased risk of discontinuation include: age 10-15 years (HR=1.74), female gender (HR=1.35) and black (HR=1.50) or Hispanic (HR=1.27) race/ethnicity. Obesity was significantly associated with higher discontinuation risk (HR=1.69, 95% CI 1.19-2.40). Children residing in the north eastern US had lower risk of discontinuation (HR=0.63, 95% CI 0.53-0.75).

TABLE 9
Cox regression for baseline characteristics associated
with TTD of somatropin with 30-mo follow-up

| | Unadjusted | Adjusted |
|---|---|---|
| Characteristics | HR (95% CI) | HR (95% CI) |
| Age | p < 0.0001 | p < 0.0001 |
| 3-9 years old | Ref | Ref |
| 10-15 years old | 1.68 (1.48-1.91) | 1.74 (1.53-1.98) |
| Gender | p < 0.0001 | p < 0.0001 |
| Male | Ref | Ref |
| Female | 1.25 (1.12-1.39) | 1.35 (1.21-1.50) |
| Region | p < 0.0001 | p < 0.0001 |
| Midwest | Ref | Ref |
| Northeast | 0.65 (0.55-0.78) | 0.63 (0.53-0.75) |
| South | 1.10 (0.97-1.24) | 1.05 (0.93-1.19) |
| West | 0.89 (0.74-1.06) | 0.91 (0.76-1.09) |
| Unknown | 3.98 (0.99-16.01) | 3.75 (0.93-15.18) |
| Ethnicity | p < 0.0001 | p < 0.0003 |
| White | Ref | Ref |
| Black | 1.62 (1.28-2.05) | 1.50 (1.18-1.90) |
| Asian | 0.83 (0.63-1.09) | 0.88 (0.67-1.16) |
| Hispanic | 1.32 (1.13-1.54) | 1.27 (1.09-1.49) |
| Unknown | 0.97 (0.84-1.13) | 0.96 (0.82-1.11) |
| Obesity | p < 0.0006 | p < 0.0034 |
| Non-obese | Ref | Ref |
| Obese | 1.85 (1.31-2.63) | 1.69 (1.19-2.40) |

Conclusions: Suboptimal somatropin adherence increases over time among all demographic subgroups. The risk of discontinuation of somatropin was higher among children>10 years of age, females, children of black or Hispanic ethnicity and obese children. Over 40% of patients discontinued somatropin therapy among all pGHD patients followed for at least 3 months. Half of these who discontinued the therapy discontinued within 1.2 years. Suboptimal adherence and discontinuation of daily growth hormone (dGH) reflect treatment challenges with current standard of care.

### Example 10

Clinical Efficacy and Safety of Once-Weekly Somatrogon Compared with Daily Dosing of Genotropin® in GH-Naïve Children with GHD Following 12 Months of Treatment

Based on results of a phase 2 dose-finding study by Zelinska (J. Clin. Endocrin. Metab. (2017) 102(5):1578-1587) a 12-month, open-label, multicenter, randomized, active-controlled, parallel-group, phase 3 study was initiated to evaluate whether somatrogon administered once weekly (0.66 mg/kg/week) was non-inferior to Genotropin® administered once daily in prepubertal children with growth hormone deficiency (GHD). This Example provides the clinical efficacy and safety of once-weekly somatrogon compared with once-daily dosing of Genotropin® in GH-naïve children with GHD following 12 months of treatment.

### Methods

1. Study Design and Treatment

This study was a 12-month, open-label, multicenter, randomized, active-controlled, parallel-group, phase 3 study comparing the safety and efficacy of somatrogon administered once-weekly to Genotropin® administered once-daily in prepubertal children with GHD who were GH-treatment naïve. This study was conducted from April 2017 to August 2019 at 83 sites in 21 countries (Argentina, Australia, Belarus, Bulgaria, Canada, Colombia, Georgia, Greece, India, Israel, Mexico, New Zealand, Poland, Russian Federation, Spain, Republic of Korea, Taiwan, Turkey, Ukraine, the United Kingdom, and United States).

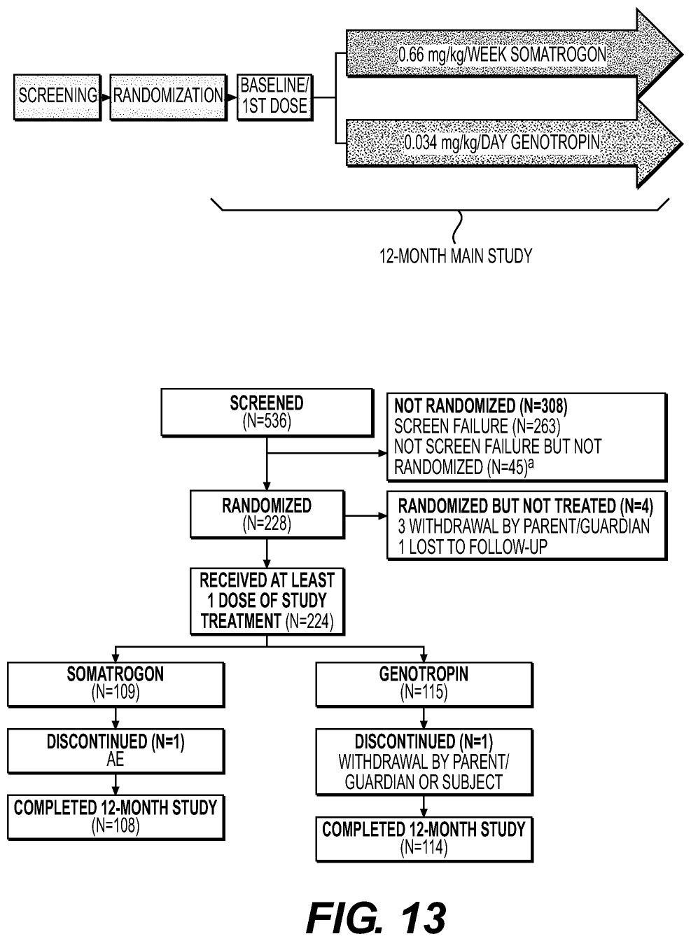

Following a 12-week screening period, subjects were randomized 1:1 (stratified according to region, GH peak levels, and chronological age) using the Interactive Web Response Technology system to receive subcutaneous (SC) doses of somatrogon administered once weekly (0.66 mg/kg/week) or SC doses of Genotropin® administered once daily (0.24 mg/kg/week) for 12 months (FIG. 13). The daily Genotropin® dose was selected as per recommendations from the current product label. Both treatments were administered using a single patient-use, multidose, prefilled pen (PEN) device. During the study, doses of somatrogon and Genotropin® were adjusted every 3 months based on the subject's body weight. Furthermore, a predefined dose-adjustment algorithm was followed to guide decreases in dose when repeated elevated insulin-like growth factor (IGF-1) levels were observed (>+2 SD score [SDS]). Subjects who completed the 12-month main study were eligible to participate in a single-arm, long-term OLE. During the OLE period, subjects who received somatrogon in the main study continued the same treatment and subjects who received Genotropin® in the main study were switched to somatrogon (0.66 mg/kw/week) (See Example 2).

The dose-adjustment algorithm was followed based on two, repeated day 4 (−1) levels of IGF-1 having an standard deviation score (SDS) of >+2.0 (which may be abbreviated as >+2.0 SDS). For subjects on Genotropin®, the dose was decreased based on repeated IGF-1 levels>+2.0 SDS. Day 4 (−1) means that IGF-1 levels were measured on day 4 following the administration of somatrogon, including up to 24 hours before (i.e., between day 3 and day 4).

If a patient had an IGF-1 level>+2.0 SDS, they were requested to return for an unscheduled visit within 4-6 weeks after the >+2.0 SDS result, on day 4 (−1) post dose for somatrogon treated subjects, or on any day for Genotropin® treated subjects. If the subject's IGF-1 level was still >+2.0 SDS, the most recent dose was reduced by 15% (i.e. to 0.56 mg/kg/week for somatrogon and to 29 μg/kg/day for Genotropin®. The subject was treated with the new dose for at least 4 weeks before a subsequent IGF-1 determination could result in a further dose modification. If the next scheduled visit was less than 4 weeks after the dose reduction was effectuated, the IGF-1 result at that visit was not used for additional dose recalculation. At the time of the next visit (or during an extra, unscheduled visit which complied with the 4 week minimum time period), IGF-1 level was retested. If the IGF-1 level was still >+2.0 SDS, the dose was reduced an additional 15% to 0.48 mg/kg/week for somatrogon and to 24.7 μg/kg/day for Genotropin®. If the IGF-1 was still >+2.0 SDS following 2 dose reductions, (at least 4 weeks after second dose reduction), the Global Study medical monitor (MM) (with the assistance of the Data Safety Monitoring Board if necessary) decided on the course of treatment on an individual basis. During the LT-OLE dose reduction for IGF-1 level>+2.0 SDS was made following consultation with the Global Study MM on an individual patient basis.

The primary objective of the study was to demonstrate that 12-month HV following once-weekly somatrogon administration was noninferior to daily Genotropin® administration in children with GHD. The secondary objectives included an evaluation of the safety and tolerability of weekly somatrogon administration.

2. Subjects

Prepubertal children (boys ages 3-11 years, girls ages 3-10 years) diagnosed with GHD were eligible for enrollment in this study if they had impaired height and HV (annualized HV below the 25th percentile for chronological age [HV<−0.7 SDS]), baseline IGF-1 level≥1 SD below the age- and sex-standardized mean IGF-1 level (SDS≤−1), and had not received prior rhGH therapy. Subject height was not required to be <−2 SDS for inclusion in this study. IGF-1 levels were quantified using the same validated assay across all testing laboratories to ensure test alignment, irrespective of physical location. Diagnosis of GHD had to be confirmed using 2 different GH provocation tests (peak plasma GH level≤10 ng/mL), determined at a local or central laboratory using a validated assay (insulin tolerance test, with serum cortisol response to hypoglycemia if insulin stimulation test is chosen; Arginine test; Clonidine test; Glucagon test; or L-dopa test). Subjects with congenital causes of multiple pituitary hormone deficits were eligible but hydrocortisone and/or L-thyroxin replacement doses had to be stable for a minimum of 3 months prior to enrollment. Children in treatment for attention-deficit hypertensive disorder were also eligible if their medication was stable for at least 3 months. Additional inclusion criteria included: bone age not older than chronological age, <10 years for females and <11 years for males; normal calculated glomerular filtration rate; children with multiple hormonal deficiencies must be on stable replacement therapies (no change in dose) for other hypothalamo-pituitary-organ axes for at least 3 months prior to signing the informed consent form; and normal 46XX (karyotype for girls.

Subjects were excluded if they had any prior history of cancer or had received radiation therapy or chemotherapy. Subjects who had a body mass index (BMI)<−2 SDS (age- and sex-standardized), anti-rhGH antibodies at screening, psychosocial dwarfism, chromosomal abnormalities (including Turner syndrome, Laron syndrome, Noonan syndrome, Prader-Willi syndrome, Russell-Silver syndrome, SHOX mutations/deletions, or skeletal dysplasia), or who were born small for their gestational age (birth weight/length<−2 SDS) were also excluded from the study. Children with type 1 or type 2 diabetes mellitus were also excluded from the study if they were deemed by the investigator as not receiving standard of care, were noncompliant with their prescribed treatment, or were in poor metabolic control. Additional exclusion criteria included: receipt of other treatments that may affect growth, including anabolic/sex steroid (except for drugs for ADHD or hormone replacement therapies); requirement for glucocorticoid therapy, receiving inhaled budesonide at dose greater than 400 μg/day or equivalent; >1 closed epiphyses; HIV-positive or with advanced diseases such as AIDS or tuberculosis;
hypersensitivity to components of study medication; and short stature caused by another condition, such as celiac disease, uncontrolled primary hypothyroidism or rickets.

3. Study Assessments

Efficacy

Height measurements were performed at baseline and months 3, 6, 9, and 12 using a calibrated, wall-mounted stadiometer; 3 independent readings were recorded for each visit. Height SDS was derived from the age and sex standards from the 2000 Centers for Disease Control Growth Charts (Centers for Disease Control. Growth Charts. 2010 (last update Sep. 9, 2010) at www.cdc.gov/growthcharts/). Annualized HV was calculated as the change in height from visit 2 (baseline) to visit 6 (month 6) and visit 8 (month 12). Bone age was determined via X-ray according to the Greulich-Pyle method using a central bone age reader at screening, baseline, and month 12 (Gruelich and Pyle, Radiographic atlas of skeletal development of the hand and wrist 1^{st} ed. Palo Alto: Stanford University Press (1959) 1-272). IGF-1 measurements were obtained at the same visits as the height measurements, as well as at month 1. IGF-1 SDS was calculated using the modified least squares (LS) mean model (Bidlingmaier et al. J. Clin. Endocrinol. Metab. (2014) 99(5):1712-1721). A previously developed indirect response PK/PD model was applied to IGF-1 observations to estimate IGF-1 SDS profiles over the dosing interval (Fisher et al. Horm. Res. Paediatr. (2017) 87(5):324-332).

Safety

Safety evaluations included all AEs, concomitant medication use, treatment compliance (monitored via patient diaries), vital signs, electrocardiogram, physical examination, and laboratory assessments that consisted of: hematology, blood chemistry, glucose metabolism (fasting blood glucose, fasting insulin level, and hemoglobin A1c (HbA1c)), endocrinology (free T4 and TSH levels), IGF-1 level, immunogenicity (anti-hGH antibodies in both groups and anti-somatrogon antibodies in the somatrogon group) and urinalysis. An AE was defined as any adverse change from the baseline condition of the subject, regardless of whether it was considered related to the investigational product. All AEs were coded using the Medical Dictionary for Regulatory Activities (MedDRA v22.0) and were classified according to the MedDRA preferred term and system organ class. The intensity or severity of an AE was characterized as mild, moderate, or severe. AEs of special interest were selected from the class-based important potential identified risks relating to somatropin-containing products.

Per protocol, injection site pain was monitored with a Pain Assessment Scale from 0 (‘no hurt’) to 5 (‘hurts worse’); pain was to be reported as an AE if the subject recorded a pain severity score≥4 in the patient diary. In the somatrogon group, the severity of injection site pain after each weekly injection was recorded, whereas, in the Genotropin® group, the most-severe pain for the week was recorded (i.e., once a week) rather than after each daily injection. Furthermore, if a Genotropin®-treated subject experienced multiple instances of pain with severity≥4 during a week, only one occurrence would be recorded in the diary and therefore only 1 AE would be recorded.

Serial serum samples were collected to test for antibodies against somatrogon using qualitative, validated methods as described by Zelinska et al. (J. Clin. Endocrin. Metab. (2017) 102(5):1578-1587).

4. Adherence

Adherence to somatrogon and Genotropin® treatment was assessed according to the following method: adherence rate (number of doses administered/number of doses expected)×100 where number of doses administered was the difference between the number of expected doses and the number of missed doses.

5. Statistical Analysis

The safety analysis set consisted of all enrolled subjects who received at least 1 dose of the study treatment. The full analysis set included all randomized subjects who received at least 1 dose of the study drug. The primary study endpoint was annual HV (cm/year) following 12 months of treatment. The noninferiority of somatrogon compared with Genotropin® was concluded if the lower bound of the 2-sided 95% confidence interval (CI) for the mean treatment difference (somatrogon—Genotropin®) in the primary efficacy endpoint was ≥−1.8 cm/year.

The CI for the difference in means between the 2 treatments was derived using ANCOVA. The ANCOVA model included terms for treatment, age group, sex, peak GH level, geographic region, and baseline height SDS as covariates. Delta-adjusted pattern imputation was applied, and the imputed values were reduced by 1.8 cm/year, i.e., the noninferiority margin.

The secondary endpoints were annualized HV following 6 months of treatment, change in height SDS at 6 and 12 months (compared with baseline), and change in bone maturation after 12 months (compared with bone age at screening). These endpoints were characterized using descriptive statistics. To support the interpretation of the ANCOVA-based primary analysis, additional sensitivity analyses included using observed data, last height carried forward, and sub-group analyses were also conducted.

### Results

1. Patients and Treatment

A total of 536 subjects were screened, 228 were randomized, and 224 received at least 1 dose of study treatment (FIG. 13). Screening failures were mainly due to subject IGF-1 levels being >−1.0 SD (˜50% of screen failures) or subjects achieving a GH peak>10 ng/mL (˜25% of screen failures). One subject from the somatrogon group discontinued from the study due to injection site erythema and injection site induration, and one subject in the Genotropin® group was withdrawn from the study (FIG. 13). In all, 99% of subjects completed the study. Most subjects in the study were male (71.9%) and White (74.6%). Demographic and baseline characteristics were similar between the 2 treatment groups (Table 10).

TABLE 10
Patient demographics and baseline characteristics (safety analysis set).

| | Somatrogon | | Genotropin ® | | Total | |
|---|---|---|---|---|---|---|
| | (n = 109) | | (n = 115) | | (N = 224) | |
| Age, mean (range), y | 7.83 | (3.01-11.96) | 7.61 | (3.05-11.85) | 7.72 | (3.01-11.96) |
| Sex, n (%) | | | | | | |
| Male | 82 | (75.2) | 79 | (68.7) | 161 | (71.9) |
| Female | 27 | (24.8) | 36 | (31.3) | 63 | (28.1) |
| Race, n (%) | | | | | | |
| White | 81 | (74.3) | 86 | (74.8) | 167 | (74.6) |
| Black or African | 0 | | 2 | (1.7) | 2 | (0.9) |
| American | | | | | | |
| Asian | 24 | (22.0) | 21 | (18.3) | 45 | (20.1) |
| American Indian or | 1 | (0.9) | 0 | | 1 | (0.4) |
| Alaska Native | | | | | | |
| Native Hawaiian or | 0 | | 1 | (0.9) | 1 | (0.4) |
| Other Pacific Islander | | | | | | |
| Other | 3 | (2.8) | 5 | (4.3) | 8 | (3.6) |
| Height SDS | | | | | | |
| Mean (SD) | −2.94 | (1.29) | −2.78 | (1.27) | −2.86 | (1.28) |
| Weight SDS | | | | | | |
| Mean (SD) | −2.66 | (2.00) | −2.41 | (1.50) | −2.53 | (1.76) |
| BMI, SDS | | | | | | |
| Mean (SD) | −0.28 | (1.04) | −0.20 | (1.01) | −0.24 | (1.02) |
| Peak GH level group, n (%) | | | | | | |
| ≤3 ng/mL | 22 | (20.18) | 21 | (18.26) | 43 | (19.20) |
| >3 ng/mL to ≤7 ng/mL | 53 | (48.62) | 56 | (48.70) | 109 | (48.66) |
| >7 ng/mL | 34 | (31.19) | 38 | (33.04) | 72 | (32.14) |
| Peak GH (ng/dL) | | | | | | |
| n | 109 | | 115 | | 224 | |
| Mean (SD) | 5.45 | (2.81) | 5.76 | (2.59) | 5.61 | (2.70) |
| Range (min, max) | (0.10, 9.93) | | (0.10, 9.90) | | (0.10, 9.93) | |
| Target height, males (cm) | | | | | | |
| n | 82 | | 78 | | 160 | |
| Mean (SD) | 169.4 | (7.04) | 172.7 | (5.56) | 171.0 | (6.56) |
| Range (min, max) | (152.0, 184.9) | | (159.5, 184.5) | | (152.0, 184.9) | |
| Target height, females (cm) | | | | | | |
| n | 25 | | 35 | | 60 | |
| Mean (SD) | 159.5 | (6.26) | 156.7 | (8.82) | 157.8 | (7.92) |
| Range (min, max) | (149.8, 175.0) | | (140.4, 171.3) | | (140.4, 175.0) | |
| Bone age, years | | | | | | |
| n | 107 | | 107 | | 214 | |
| Mean (SD) | 5.46 | (2.72) | 5.19 | (2.45) | 5.33 | (2.59) |

| | | | |
|---|---|---|---|
| Range (min, max) | (1.00, 11.00) | (1.25, 11.00) | (1.00, 11.00) |

BMI, body mass index;
GH, growth hormone;
min max, minimum maximum

2. Efficacy

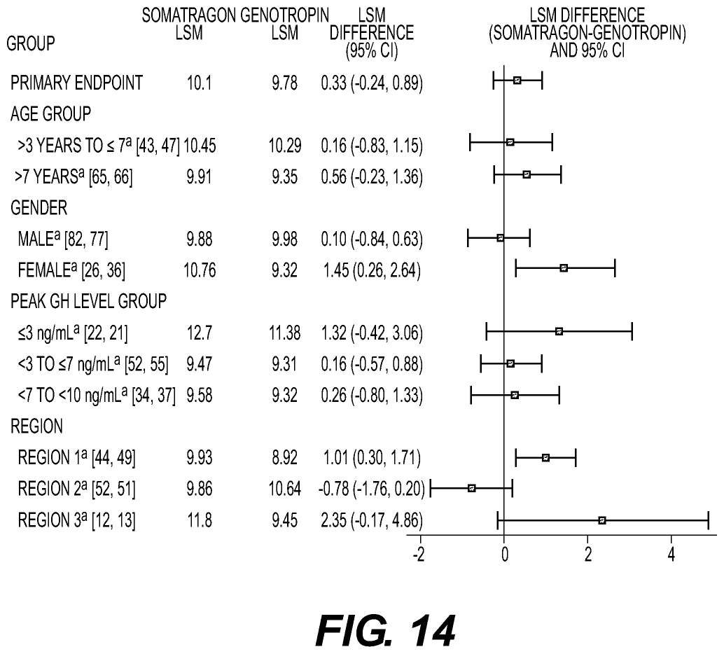

At month 12, the LS mean estimate of annual HV using the ANCOVA model was 10.10 cm/year for somatrogon and 9.78 cm/year for Genotropin®. The treatment mean difference (somatrogon—Genotropin®) was 0.33 cm (95% CI: −0.24, 0.89). As the lower bound of the 2-sided 95% CI was greater than the prespecified noninferiority margin (−1.8 cm/year), the study was considered to have met its primary objective of demonstrating that somatrogon administered once-weekly was noninferior to Genotropin® administered once-daily with respect to annual HV at 12 months in children with GHD. Results obtained using various sensitivity analyses were consistent with and supportive of the primary endpoint. The prespecified subgroup analyses comparing somatrogon and Genotropin® treatment based on age, sex, or peak GH levels showed that similar HVs were achieved in response to both treatments (FIG. 14).

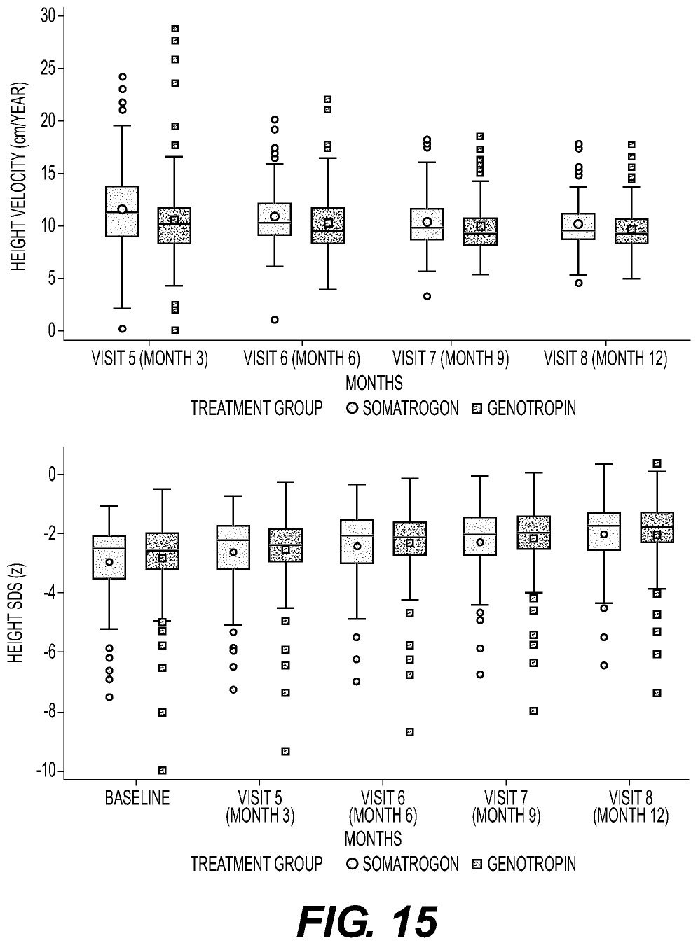

The mean annualized HV at 6 months in the somatrogon group was similar to the Genotropin® group, with LS mean estimates of 10.59 and 10.04 cm/year, respectively (LS mean treatment difference 0.55 cm [95% CI, −0.13, 1.23]). At all post-baseline visits, both treatment groups had similar HV (FIG. 15). Subjects in both the somatrogon and Genotropin® groups showed similar improvements in mean change in height SDS from baseline to 6 months (LS mean treatment difference 0.06 [95% CI, −0.01, 0.13]). Similar improvements were also observed for both treatment groups from baseline to 12 months (LS mean treatment difference 0.05 [95% CI, −0.06, 0.16]).

Individual growth responses of patients receiving somatrogon and Genotropin® demonstrated similar growth trends and variability in both groups; peak height velocity was seen at 3 months. The large variability of height velocity in both groups at 3 months was expected due to the known impact of height measurement errors in this short interval.

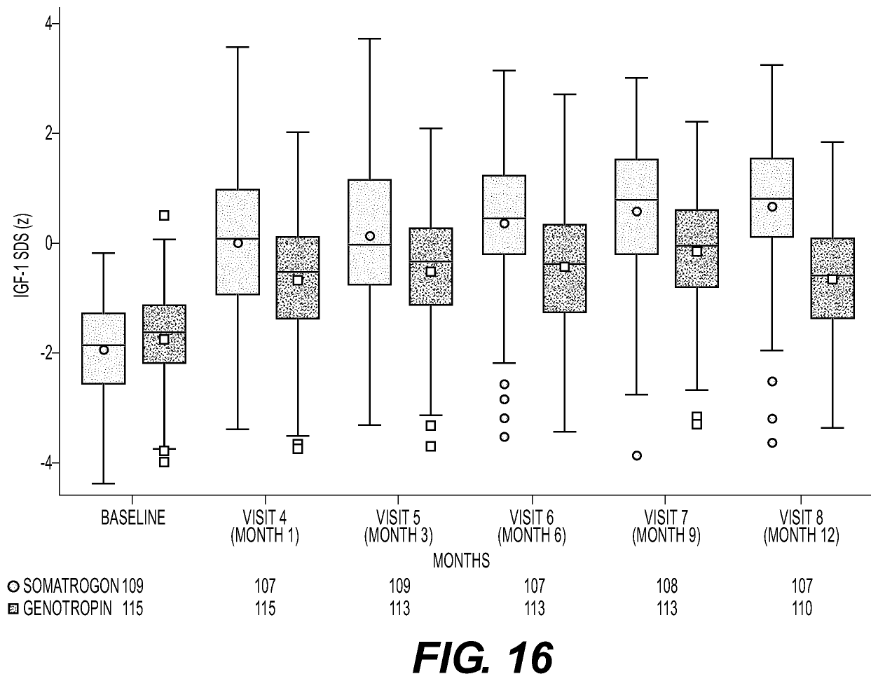

Bone maturation, assessed as change in bone age relative to change in chronological age from baseline to 12 months, was similar between treatment groups (somatrogon: 1.07; Genotropin®: 1.12). In the somatrogon group, the mean value for IGF-1 SDS approached 0 at 1 month post-baseline and was 0.65 SDS (range: −3.64 to 3.22) at 12 months post-baseline (FIG. 16). The IGF-1 SDS mean value in the Genotropin® group remained near 0 at all post-baseline visits, ranging from −0.69 to −0.16 SDS (FIG. 16).

3. Safety

The 2 treatment groups had a similar mean (SD) duration of treatment: somatrogon: 363 (32) days; Genotropin®: 355 (28) days. In all, 192 of 224 patients (85.7%) experienced a treatment-emergent AE (TEAE). The incidence of TEAEs was similar between the somatrogon (87.2%) and Genotropin® groups (84.3%) (Table 11). Most of the all-causality TEAEs experienced with somatrogon vs Genotropin® were mild (54.1% vs 60.0%) or moderate (24.8% vs 19.1%) in intensity. The incidence of severe TEAEs was 8.3% and 5.2% in the respective groups.

TABLE 11
Treatment-related adverse events (all-causality).

| | Somatrogon | | Genotropin ® | | Total | |
|---|---|---|---|---|---|---|
| Number (%) of subjects | (n = 109) | | (n = 115) | | (N = 224) | |
| Number of AEs | 868 | | 570 | | 1438 | |
| Subjects with AEs | 95 | (87.2) | 97 | (84.3) | 192 | (85.7) |
| Subjects with serious AEs | 3 | (2.8) | 2 | (1.7) | 5 | (2.2) |
| Subjects with severe AEs | 9 | (8.3) | 6 | (5.2) | 15 | (6.7) |
| Subjects discontinued from | 1 | (0.9) | 0 | | 1 | (0.4) |
| study due to AEs^{a} | | | | | | |
| Subjects discontinued study | 0 | | 0 | | 0 | |
| drug due to AE and | | | | | | |
| continued study^{b} | | | | | | |
| Subjects with dose reduced or | 3 | (2.8) | 2 | (1.7) | 5 | (2.2) |
| temporary discontinuation | | | | | | |
| due to AEs | | | | | | |

Serious AEs are based on the investigator's assessment.
^{a}Subjects who have an AE record that indicated that the AE caused the subject to be discontinued from the study.
^{b}Subjects who have an AE record that indicated that action taken with study treatment was drug withdrawn but AE did not cause the subject to be discontinued.

The most frequently reported all-causality TEAEs by MedDRA preferred term that occurred in ≥5% of subjects in any treatment group were injection site pain, nasopharyngitis, headache, pyrexia, cough, vomiting, anemia, arthralgia, bronchitis, pharyngitis, otitis media, tonsillitis, blood creatinine phosphokinase increased, oropharyngeal pain, hypothyroidism, ear pain, injection site erythema, abdominal pain upper, rhinitis, arthropod bite, and injection site pruritus (Table 12). All-causality TEAEs with >5% higher incidence in the somatrogon group than in the Genotropin® group were injection site erythema, injection site pain, and injection site pruritus (Table 12).

TABLE 12
All-causality treatment-related adverse events reported in ≥5%
of subjects in either treatment group (safety analysis set).

| | Somatrogon | | Genotropin ® | | Total | |
|---|---|---|---|---|---|---|
| Number (%) of subjects | (n = 109) | | (n = 115) | | (N = 224) | |
| With any AE | 92 | (84.4) | 90 | (78.3) | 182 | (81.3) |
| Injection site pain | 43 | (39.4) | 29 | (25.2) | 72 | (32.1) |
| Nasopharyngitis | 25 | (22.9) | 29 | (25.2) | 54 | (24.1) |
| Headache | 18 | (16.5) | 25 | (21.7) | 43 | (19.2) |
| Pyrexia | 18 | (16.5) | 16 | (13.9) | 34 | (15.2) |
| Cough | 9 | (8.3) | 9 | (7.8) | 18 | (8.0) |
| Vomiting | 8 | (7.3) | 9 | (7.8) | 17 | (7.6) |
| Anemia | 7 | (6.4) | 7 | (6.1) | 14 | (6.3) |
| Arthralgia | 5 | (4.6) | 8 | (7.0) | 13 | (5.8) |
| Bronchitis | 3 | (2.8) | 9 | (7.8) | 12 | (5.4) |
| Pharyngitis | 7 | (6.4) | 5 | (4.3) | 12 | (5.4) |
| Otitis media | 4 | (3.7) | 7 | (6.1) | 11 | (4.9) |
| Tonsillitis | 5 | (4.6) | 6 | (5.2) | 11 | (4.9) |
| Blood creatine | 2 | (1.8) | 8 | (7.0) | 10 | (4.5) |
| phosphokinase increased | | | | | | |
| Oropharyngeal pain | 6 | (5.5) | 4 | (3.5) | 10 | (4.5) |
| Hypothyroidism | 7 | (6.4) | 3 | (2.6) | 10 | (4.5) |
| Ear pain | 2 | (1.8) | 7 | (6.1) | 9 | (4.0) |
| Injection site erythema | 9 | (8.3) | 0 | | 9 | (4.0) |
| Abdominal pain upper | 2 | (1.8) | 6 | (5.2) | 8 | (3.6) |
| Rhinitis | 6 | (5.5) | 1 | (0.9) | 7 | (3.1) |
| Arthropod bite | 6 | (5.5) | 1 | (0.9) | 7 | (3.1) |

| | | | | | |
|---|---|---|---|---|---|
| Injection site pruritus | 6 | (5.5) | 0 | 6 | (2.7) |

Most events of injection site pain were mild or moderate in severity for subjects in both treatment groups. Eight subjects reported severe injection site pain (somatrogon: n=5 [4.6%]; Genotropin®: n=3 [2.6%]). Most episodes of injection site pain occurred during the first 6 months of treatment. For some subjects, however, injection site pain was reported throughout the study, usually with mild or decreasing severity.

Both treatment groups had a similarly low incidence of SAEs (somatrogon: 2.8%; Genotropin®: 1.7%) and none were considered related to the study treatment (Table 10). No deaths occurred during the study and only 1 subject (somatrogon group) discontinued from the study due to an AE (injection site erythema and injection site induration) (Table 10). The incidence of dose reductions or temporary study drug discontinuations due to an AE was low overall (2.2% [n=5]) and similar between somatrogon (2.8% [n=3]) and Genotropin® (1.7% [n=2]). No TEAEs led to a dose reduction of the study drug.

Overall, 29 subjects experienced IGF-1 levels>2 SDS sometime during the study (somatrogon: n=26; Genotropin®; n=3). Of the 26 subjects in the somatrogon group, 14 experienced persistent IGF-1 level>2 SDS (i.e., 2 consecutive measurements with a SDS>2), which resulted in dose reductions for 12 of these subjects. Overall, subjects with 2 consecutive IGF-1 values>2 SDS had a comparable safety profile as those without consecutive IGF-1 elevations.

Using the data collected, a PK/PD analysis was performed to simulate IGF-1 profiles for each of the study subjects and to estimate the mean IGF-1 SDS over the dosing interval, regardless of when the sample had been collected. Among somatrogon-treated subjects, 10 of 535 (1.9%) samples that corresponded to mean IGF-1 SDS over the dosing interval were >2. These 10 instances of mean IGF-1 SDS>2 occurred in 3 subjects and no subject had a mean IGF-1 SDS≥3. The use of PK/PD modeling as a tool to estimate IGF-1 SDS profiles over the dosing interval confirmed that samples collected close to 96 hours after dose administration represent the mean IGF-1 SDS over the week between doses. The PK/PD modeling also confirmed that samples collected 48-72 hours after dose administration represent peak IGF-1 SDS over the week between doses.

Glucose and HbA1c levels rose discretely during the 12-month period in both treatment groups, and values remained within the normal range. No clinically meaningful differences in thyroid function, lipids, vital assessments, or physical examinations were observed between subjects treated with somatrogon or Genotropin®. There were no cases of drug-induced liver injury in any subjects.

4. Immunogenicity

Among 109 Somatrogon-treated subjects, 84 subjects (77.1%) tested positive for antidrug antibodies (ADAs) at any time during the 12-month study period. Among 115 Genotropin®-treated subjects, 18 (15.6%) tested positive for ADAs. Post hoc analyses comparing clinical endpoint results to ADA status indicated that the presence of ADAs did not have an effect on overall safety (e.g. adverse events) or efficacy (e.g. growth rate) during the main study. Further, no ADAs had evidence of neutralizing activity on safety or efficacy.

5. Adherence

The overall adherence rate for this study was 99.6%, with very high adherence observed in both the somatrogon (99.4%) and Genotropin® (99.7%) groups. The lowest adherence rate observed for an individual patient was 87.5% in the somatrogon group and 91.5% in the Genotropin® groups.

### Discussion

The objective of this Example was to evaluate the safety and efficacy of somatrogon administered once-weekly compared with Genotropin® administered once-daily in prepubertal children with GHD. Conducted in 21 countries, the primary objective was met, demonstrating that once-weekly treatment with somatrogon was non-inferior to daily treatment with Genotropin®. The LS mean estimate of annual HV at 12 months was 10.10 cm/year for somatrogon and 9.78 cm/year for Genotropin®. The somatrogon and Genotropin® groups were also similar with regards to the mean annualized HV at 6 months, improvements in mean change in height SDS from baseline to 6 and 12 months, and mean change in bone maturation at 12 months. The efficacy of somatrogon administered once-weekly was consistent with what was observed for the highest dose group (somatrogon 0.66 mg/kg/week) in the previous phase 2 study, as reported by Zelinska et al, with the mean annualized HV in the somatrogon group similar to that for the Genotropin® group (11.9 cm/year and 12.5 cm/year, respectively) and a similar improvement observed in height SDS (J. Clin. Endocrinol. Metab. (2017) 102(5):1578-1587).

The safety and tolerability of somatrogon administered once-weekly was similar to that of Genotropin® administered once-daily in prepubertal children with GHD. The incidence of SAEs was low for both treatments (<3%), and no SAEs were considered to be related to study treatment. There were no deaths reported during the study. Both treatment groups also had a similar incidence of all-causality TEAEs (somatrogon: 87.2%; Genotropin®: 84.3%), most of which were mild or moderate in intensity. The most commonly reported all-causality TEAE was injection site pain, which was reported by 39.4% and 25.2% of subjects in the somatrogon and Genotropin® groups, respectively. The between-group difference in the number of reports of injection site pain did not appear to be due to a difference in age or injection site location and may have been a result of differences in the way that injection site pain was recorded in the two treatment groups, as outlined in the Methods above. The modest increases in glucose and HbA1c levels observed have also been described in previous studies (Ciresi et al. J. Endocrinol. Invest. (2015) 38(12):1301-1307; Witkowska-Sedek et al. J. Physiol. Pharmacol. (2018) 69(2)); however, the resulting values in this study were still within the normal range. ADAs were reported in 84 subjects (77.1%) during the study and the presence of ADAs did not appear to have an effect on safety or efficacy.

The incidence of temporary discontinuations of the study drug due to an AE was low (<3%) in both study groups. Only 1 subject from the somatrogon group permanently discontinued from the study, due to injection site erythema and injection site induration (moderate severity, treatment-related). The tolerability of once-weekly somatrogon and once-daily Genotropin® was underscored by the fact that of the 224 subjects who enrolled in the study, 222 (99%) completed the main study. Furthermore, of these 222 subjects, 212 (somatrogon: n=104 [95%]; Genotropin®: n=108 [94%]) chose to enrol into the optional OLE. The safety findings from this study were similar to that reported in the previous phase 2 study. The incidence of AEs in this study was similar between the somatrogon (69.0%) and Genotropin® (72.7%) groups and was similar to what was reported in the phase 2 study (Zelinska et al. J. Clin. Endocrinol. Metab. (2017) 102(5):1578-1587).

As stated above, the safety profile of the subjects with 2 consecutive IGF-1 values>2 SDS was similar to that of subjects without elevated IGF-1 values. There is currently little clinical evidence to suggest that high IGF-1 levels increase the risk of adverse events (Allen et al. Eur. J. Endocrin. (2016) 172(2):1-9). With daily administered rhGH products, the time of sample collection for IGF-1 measurement is not a concern as fluctuations over the 24-hour dosing interval are modest and as such, all sampling times provide reasonable estimates of the average IGF-1 SDS. However, interpretation of IGF-1 SDS for somatrogon needs to consider the timing of sample collection due to the significant peak trough fluctuation over the dosing interval (Fisher et al. Horm. Res. Paediatr. (2017) 87(5):324-332; Bidlingmaier and Schilbach, J. Clin. Endocrin. Metab. (2021) 106(5):e2367-e2369). With weekly dosing of long-acting somatrogon, samples collected 2 or 3 days post-dose provided good estimates of peak IGF-1. Although collecting samples approximately 96 hours (4 days) post somatrogon dose provided an accurate estimate of the mean IGF-1 SDS over the dosing interval, in real-world practice, IGF-1 SDS monitoring at any day post dosing requires the use of PK/PD-generated models for all long-acting GH products (Bidlingmaier and Schilbach, J. Clin. Endocrin. Metab. (2021) 106(5): e2367-e2369).

Given the similarity of the efficacy, safety, and tolerability of long-acting GH products compared with once-daily Genotropin®, the use of long-acting GH products such as somatrogon for the treatment of pediatric GHD may address some of the issues with adherence and persistence currently associated with daily rhGH treatment, without compromising patient health and development. Poor adherence to treatment and early cessation have been identified as key issues associated with daily rhGH treatment (Cutfield et al., PloS One (2011) 6(1):e16223; Hughes et al., Growth Horm. IGF Res. (2016) 29:63-70; Kremidas et al., J. Pediatr. Nurs. (2013) 28(1):55-63). In addition to reduced efficacy, poor adherence can also result in substantial costs being borne for unused treatment (Cutfield et al., PloS One (2011) 6(1):e16223). A recent study of pediatric patients, caregivers, and adult patients showed a strong preference for a less-frequent injection schedule for the treatment of GHD (McNamara et al., (2020) 14:781-793). The use of somatrogon which does not require daily administration alleviates some of the burdens described above, leading to improved adherence and treatment benefit.

This Example demonstrates that the efficacy of somatrogon administered once-weekly was non-inferior to Genotropin® administered once-daily for the treatment of prepubertal children with GHD. Once weekly somatrogon resulted in a robust and sustained increase in HV compared with daily GH treatment, while maintaining IGF-1 and bone age advancement within the normal range. Long-acting somatrogon and daily GH had similar safety and tolerability profiles. Compared with Genotropin® administered once-daily, the less-frequent injection schedule afforded by somatrogon administered once-weekly improved poor adherence and quality of life, which are key unmet needs in this pediatric population (Brod et al. Patient (2017) 10(5):653-666).

Although the disclosed teachings have been described with reference to various applications, methods, and compositions, it will be appreciated that various changes and modifications can be made without departing from the teachings herein and the claimed invention below. The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, textbooks, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

We claim:

1. A method of treating a growth hormone deficiency (GHD) in a subject in need thereof, the method comprising:
administering a long-acting recombinant human growth hormone (rhGH) comprising the amino acid sequence of SEQ ID NO:2 to the subject at an initial dose level;
taking at least two measurements of an insulin growth factor 1 (IGF-1) level in the subject; and
administering the long-acting rhGH to the subject at a modified dose level that is about 15% lower than the initial dose level when the IGF-1 level in the subject on two consecutive measurements taken 4 to 6 weeks apart each has a standard deviation score (SDS) of greater than positive 2 (>+2).

2. The method of claim 1, wherein the long-acting rhGH is administered once a week at the initial dose level or at the modified dose level.

3. The method of claim 1, wherein the subject is a pediatric subject.

4. The method of claim 1, wherein the initial dose level is about 0.66 milligrams (mg) per kilogram (kg) of body weight per week.

5. The method of claim 1, further comprising taking at least one additional measurement of an IGF-1 level in the subject at least 4 weeks after administering the modified dose level.

6. The method of claim 5, further comprising administering the long-acting rhGH to the subject at a further modified dose level when the at least one additional measurement of an IGF-1 level in the subject has an SDS of >+2.

7. The method of claim 6, wherein the long-acting rhGH is administered once a week at the further modified dose level.

8. The method of claim 6, wherein the further modified dose level is about 15% lower than the modified dose level.

9. A method of treating a growth hormone deficiency (GHD) in an adult subject in need thereof, the method comprising:
administering a long-acting recombinant human growth hormone (rhGH) comprising the amino acid sequence of SEQ ID NO:2 to the subject at an initial dose level;
taking at least one measurement of an insulin growth factor 1 (IGF-1) level in the subject; and
administering the long-acting rhGH to the subject at a modified dose level that is about 0.5 milligrams/week (mg/week) or about 0.75 mg/week lower than the initial dose level when the IGF-1 level in the subject has a standard deviation score (SDS) value of greater than positive 1.5 (>+1.5), or that is about 1.0 mg/week or about 1.5 mg/week higher than the initial dose level when the IGF-1 level in the subject has an SDS of less than negative 0.5 (<−0.5).

10. The method of claim 9, wherein the long-acting rhGH is administered once a week at the initial dose level or at the modified dose level.

11. The method of claim 9, wherein the initial dose level ranges from about 1 mg/week to about 5 mg/week.

12. The method of claim 9, wherein the initial dose is: about 2.5 mg/week for a male 50 years of age or less, about 2.0 mg/week for a male greater than 50 years of age, about 3.0 mg/week for a female not on oral estrogen who is 50 years of age or less, about 2.5 mg/week for a female not on oral estrogen who is greater than 50 years of age, about 4.0 mg/week for a female on oral estrogen who is 50 years of age or less, or about 3.5 mg/week for a female on oral estrogen who is greater than 50 years of age.

13. The method of claim 9, wherein the IGF-1 level is measured in serum or plasma.

14. The method of claim 9, wherein the IGF-1 level is measured at day 3 to day 4 after administering the long-acting rhGH at an initial dose level.

15. The method of claim 9, further comprising taking at least one additional measurement of an IGF-1 level in a subject after administering the long-acting rhGH at the modified dose level.

16. The method of claim 15, further comprising administering the long-acting rhGH to the subject at a further modified dose level when the at least one additional measurement of an IGF-1 level in the subject has an SDS of greater than positive 1.5 (>+1.5).

17. The method of claim 16, wherein the further modified dose level is about 0.5 mg/week lower or about 0.75 mg/week lower than the modified dose level.

18. The method of claim 15, further comprising administering the long-acting rhGH to the subject at a further modified dose level when the at least one additional measurement of an IGF-1 level in the subject has an SDS of less than negative 0.5 (<−0.5).

19. The method of claim 18, wherein the further modified dose level is about 1.0 mg/week higher or about 1.5 mg/week higher than the modified dose level.

20. The method of claim 9, wherein the subject's trunk fat mass is decreased, lean body mass is increased, trunk fat mass as a percentage of total fat mass is decreased, IGF-1 levels are normalized, or any combination thereof after said treating.

21. A method of treating a growth hormone deficiency (GHD) in an adult subject in need thereof, the method comprising:
administering a long-acting recombinant human growth hormone (rhGH) comprising the amino acid sequence of SEQ ID NO:2 to the subject at an initial dose level;
monitoring the subject for an adverse event; and
administering the long-acting rhGH to the subject at a modified dose level,
wherein the modified dose level is 25% lower than the initial dose level if the adverse event is moderate, or
wherein the modified dose level is 50% lower than the initial dose level if the adverse event is severe.

22. The method of claim 21, wherein the adverse event is edema, hypertension, carpal tunnel, glucose, or a combination thereof.

23. A method of treating a growth hormone deficiency (GHD) in a subject in need thereof, the method comprising:
selecting the subject with GHD, wherein the subject has previously received a once daily recombinant human growth hormone (once daily rhGH) therapy; and
administering a therapeutically effective amount of a long-acting recombinant human growth hormone (long-acting rhGH) to the subject,
so that the efficacy of the long-acting rhGH in the subject is comparable to an efficacy of the long-acting rhGH in a subject with GHD who has previously received only the long-acting rhGH and has not previously received the once daily rhGH therapy.

24. The method of claim 23, wherein the once daily rhGH is somatropin, somatrem, a somatropin biosimilar, or a somatrem biosimilar.

25. The method of claim 23, wherein the efficacy is assessed by measuring one or more of:
mean height velocity, gain in height standard deviation score (SDS), body mass index, bone maturation, insulin growth factor-1 (IGF-1) standard deviation score (SDS), insulin-like growth factor binding protein 3 (IGFBP-3) SDS, pubertal status changed from Tanner 1, mean glucose levels, hemoglobin A1c (HbA1c) levels, thyroid function, and cholesterol values.

26. The method of claim 23, wherein the long-acting rhGH is administered according to a dosage regimen comprising subcutaneous administration of 0.66 milligrams (mg) per kilogram (kg) body weight once weekly at any time of day.

27. The method of claim 23, wherein the subject had received a once daily recombinant human growth hormone for at least three months.

28. The method of claim 23, wherein the subject is 3 to 15 years old.

29. The method of claim 23, wherein the subject has one or more of the following: isolated growth hormone deficiency (GHD), GH insufficiency as part of multiple pituitary hormone deficiency, pediatric GHD, or Prader-Willi Syndrome.

30. The method of claim 23, wherein the long-acting rhGH is administered subcutaneously in the abdomen, thighs, buttocks, or upper arm.


SEQUENCE LISTING (3 sequences)

SEQ ID NO: 1 - protein, 217 aa, Artificial Sequence
  Synthetic Construct
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - protein, 275 aa, Artificial Sequence
  long acting recombinant human growth hormone
SSSSKAPPPSLPSPSRLPGPSDTPILPQFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSSSSKAPPPSLPSPSRLPGPSDTPILPQSSSSKAPPPSLPSPSRLPGPSDTPILPQ

SEQ ID NO: 3 - protein, 28 aa, Artificial Sequence
  Synthetic Construct
SSSSKAPPPSLPSPSRLPGPSDTPILPQ